(19) United States
(12) Patent Application Publication
(10) Pub. No.: US 2017/0016004 A1
(43) Pub. Date: Jan. 19, 2017

(54) DDX5 AND ASSOCIATED NON-CODING RNAs AND MODULATION OF TH17 EFFECTOR FUNCTION

(71) Applicants: Dan R. Littman, New York, NY (US); Wendy Huang, New York, NY (US)

(72) Inventors: Dan R. Littman, New York, NY (US); Wendy Huang, New York, NY (US)

(21) Appl. No.: 15/165,727

(22) Filed: May 26, 2016

Related U.S. Application Data
(60) Provisional application No. 62/168,182, filed on May 29, 2015.

(51) Int. Cl. C12N 15/113; C12Q 1/34

(52) U.S. Cl. CPC C12N 15/1137; C12Q 1/34; C12N 2310/11; G01N 2500/02; G01N 2500/20

(57) ABSTRACT

Methods for screening to identify agents capable of modulating DDX5 polypeptide activity are encompassed herein as are methods for using such agents to treat subjects afflicted with T_{H}17-mediated inflammatory conditions and autoimmune diseases including, without limitation, Crohn's disease, ulcerative colitis, multiple sclerosis, rheumatoid arthritis, and psoriasis and methods for using such agents to treat subjects afflicted with cancer.

### CROSS REFERENCE TO RELATED APPLICATION

This application claims priority under 35 USC §119(e) from U.S. Provisional Application Ser. No. 62/168,182, filed May 29, 2015, which application is herein specifically incorporated by reference in its entirety.

### GOVERNMENTAL SUPPORT

The research leading to the present invention was funded in part by Institutional NRSA T32 CA009161_Levy and National Institutes of Health Grant Nos. NIH R01AI080885 and NIH R01DK103358. The United States government has certain rights in the invention.

### FIELD OF THE INVENTION

The present invention pertains to the fields of immunology and inflammation and autoimmunity and regulation thereof. More particularly, the invention relates to in vitro screening methods directed to identifying agents capable of modulating T_{H}17 cell activity and in vitro and in vivo methods directed to modulating T_{H}17 cell activity. As described herein, agents that modulate (inhibit or enhance) interactions between and among RAR-related Orphan Receptor-gamma (RORγt), RNA helicase DEAD-box protein 5 (DDX5), and Nuclear encoded Rmrp, RNA component of Mitochondria RNA-processing endoribonuclease (RNase MRP; also refered to herein as Rmrp) are envisioned as exemplary modulators of T_{H}17 cell activity and may be used to advantage as therapeutic agents for treating inflammatory and autoimmune disorders and cancers.

### BACKGROUND OF THE INVENTION

Several publications and patent documents are referenced in this application in order to more fully describe the state of the art to which this invention pertains. The disclosure of each of these publications and documents is incorporated by reference herein.

T_{H}17 cells are CD4+ lymphocytes that help to protect mucosal epithelial barriers against bacterial and fungal infections (1), and are also important in multiple autoimmune diseases (2-7). The T_{H}17 cell differentiation program is defined by the induced expression of RORγt (2), a sterol ligand-regulated nuclear receptor that focuses the activity of a cytokine-regulated transcriptional network upon a subset of key genomic target sites, including genes encoding the signature T_{H}17 cytokines (interleukin (IL)-17A, IL-17F and IL-22) as well as IL-23R, IL-1R1 and CCR6 (8). In mouse models, attenuation of RORγt activity results in protection from experimental autoimmune encephalomyelitis (EAE), T-cell-transfer-mediated colitis and collagen-induced arthritis (2-5). Like other nuclear receptors, RORγt interaction with its ligands results in recruitment of coactivators at regulated genomic loci (9).

### SUMMARY OF INVENTION

As described herein, the present inventors identified two new RORγt partners in T_{H}17 cells, an RNA helicase and a long noncoding RNA (lncRNA), which together associate with RORγt to confer target-locus-specific activity in enabling the T-cell effector program. The RNA helicase DDX5 functions in multiple cellular processes (10), including transcription and ribosome biogenesis (11-17), in both a helicase-activity-dependent and -independent manner. The lncRNA Rmrp, RNA component of mitochondria RNA processing endoribonuclease (also known as RNase MRP), is highly conserved between mouse and human and is essential for early mouse development (18). Rmrp was first identified as a component of the RNase MRP complex that cleaves mitochondrial RNAs (19). In yeast, the RMRP1 gene contributes to ribosomal RNA processing and regulates messenger RNA (mRNA) degradation (20). In humans, mutations located in evolutionarily conserved nucleotides at the promoter or within the transcribed region of RMRP result in cartilage-hair hypoplasia (CHH), a rare autosomal recessive disorder characterized by early childhood onset of skeletal dysplasia, hypoplastic hair, defective immunity, predisposition to lymphoma and neuronal dysplasia of the intestine (21, 22). Immune deficiency in CHH patients is associated with recurrent infections, haematological abnormalities and autoimmune pathologies in the joints and kidneys (23). The precise mechanisms by which Rmrp functions in the immune system have yet to be determined. Here the present inventors show that the helicase activity of DDX5 mediates Rmrp-dependent binding to RORγt and recruitment to a subset of chromatin target sites, thus controlling the differentiation of T_{H}17 cells at steady state and in animal models of autoimmunity.

In light of results presented herein, the present inventors describe DDX5, together with the lncRNA Rmrp, as novel transcriptional coactivators for RORγt. The helicase-activity of DDX5 is shown herein to mediate their joint recruitment to chromatin sites occupied by RORγt. Intriguingly, a targeted Rmrp mutation in mice, corresponding to one in CHH patients, abrogated its ability to potentiate a subset of the RORγt transcription program in T_{H}17 cells.

In accordance with these findings, a method for screening to identify a modulator of RNA helicase DEAD-box protein 5 (DDX5) activity is presented, the method comprising: (a) providing a polypeptide mixture comprising RORγt polypeptide or a fragment thereof, DDX5 polypeptide, ribonucleic acid (RNA) component of Mitochondria RNA-processing endoribonuclease (Rmrp) and at least one candidate modulator agent; and (b) detecting RORγt/DDX5/Rmrp complex formation or RORγt fragment/DDX5/Rmrp complex formation in the presence of the candidate modulator agent and comparing that to RORγt/DDX5/Rmrp complex formation or RORγt fragment/DDX5/Rmrp complex formation in the absence of the candidate modulator agent, wherein a change in RORγt/DDX5/Rmrp complex formation or RORγt fragment/DDX5/Rmrp complex formation in the presence of the candidate modulator agent relative to that detected in the absence of the candidate modulator agent indicates that the at least one candidate modulator agent is a modulator of DDX5 polypeptide activity.

In another aspect, a method for screening to identify a modulator of DDX5 polypeptide activity is presented, the method comprising: (a) providing a polypeptide mixture comprising DDX5 polypeptide, Rmrp, and at least one candidate modulator agent; and (b) detecting DDX5/Rmrp complex formation in the presence of the candidate modulator agent and comparing that to DDX5/Rmrp complex formation in the absence of the candidate modulator agent, wherein a change in DDX5/Rmrp complex formation in the presence of the candidate modulator agent relative to that detected in the absence of the candidate modulator agent indicates that the at least one candidate modulator agent is a modulator of DDX5 polypeptide activity.

In yet another aspect, a method for screening to identify an enhancer of DDX5 polypeptide activity is presented, the method comprising: (a) providing a polypeptide mixture comprising RORγt polypeptide or a fragment thereof, DDX5 polypeptide, and at least one candidate modulator agent; and (b) detecting RORγt/DDX5/Rmrp complex formation or RORγt fragment/DDX5 complex formation in the presence of the candidate modulator agent and comparing that to RORγt/DDX5/Rmrp complex formation or RORγt fragment/DDX5 complex formation in the absence of the candidate modulator agent, wherein formation of RORγt/DDX5/Rmrp complexes or RORγt fragment/DDX5 complexes in the presence of the candidate modulator agent relative to that detected in the absence of the candidate modulator agent indicates that the at least one candidate modulator agent is an enhancer of DDX5 polypeptide activity.

In a particular embodiment of methods described herein, the change detected in the presence of the candidate modulator agent is a decrease in RORγt/DDX5/Rmrp complex formation or RORγt fragment/DDX5/Rmrp complex formation or DDX5/Rmrp complex formation, thereby identifying the candidate modulator agent as an inhibitor of DDX5 polypeptide activity.

In another particular embodiment, the change detected in the presence of the candidate modulator agent is an increase in RORγt/DDX5/Rmrp complex formation or RORγt fragment/DDX5/Rmrp complex formation or DDX5/Rmrp complex formation, thereby identifying the candidate modulator agent as an enhancer of DDX5 polypeptide activity.

In an embodiment of the method, the screening is performed in a cell-based assay or in vitro assay comprising purified components. Cell-based assays may comprise naive CD4+ T cells polarized under T_{H}17 inducing conditions or a cell or cells engineered to express an exogenous gene operably linked to a RORγt-dependent responsive regulatory element.

In a particular embodiment of the method, the formation of RORγt/DDX5/Rmrp complexes or RORγt fragment/DDX5/Rmrp complexes, DDX5/Rmrp complexes, or RORγt//DDX5/Rmrp complexes or RORγt fragment/DDX5 complexes detected in the presence or absence of the candidate modulator agent is measured by a change in expression of at least one transcriptional readout in the cell-based assay or the in vitro assay comprising purified components. Exemplary transcriptional readouts comprise RORγt-dependent loci selected from the group consisting of Il17a, Il17f, and Il22 or an exogenous reporter gene operably linked to a RORγt-dependent responsive regulatory element. The change in expression of at least one transcriptional readout may be measured by at least one of a change in Il17a, Il17f, and Il22 mRNA levels and/or by at least one of a change in IL17A, IL17F, and IL22 polypeptide expression or a change in expression of an exogenous polypeptide encoded by the exogenous reporter gene.

In a particular embodiment, the polypeptide mixture comprises MgCl₂ and Adenosine Triphosphate (ATP) and has a physiological pH of about 7.5.

In another particular embodiment, at least one of the RORγt polypeptide or RORγt fragment and the DDX5 polypeptide comprises a tag moiety. An exemplary RORγt fragment comprises the ligand binding domain of the RORγt polypeptide.

In an embodiment of the method, the candidate modulator agent is a small organic molecule, a peptide, or a nucleic acid. Such nucleic acids may be single-strand or double-strand DNA or RNA. An exemplary single-strand DNA envisioned herein is an anti-sense oligonucleotide. In a particular embodiment thereof, the anti-sense oligonucleotide is specific for Rmrp or DDX5.

In another aspect, a method for treating a mammalian subject afflicted with an inflammatory condition or autoimmune disease associated with T_{H}17 cell mediated pathology is envisioned, the method comprising administering an inhibitor of DDX5 polypeptide activity identified using methods described herein to the subject, wherein the inhibitor of DDX5 polypeptide activity reduces T_{H}17 cell activity in the subject and thereby treats the mammalian subject. In an embodiment thereof, the mammalian subject is afflicted with Crohn's disease, ulcerative colitis, multiple sclerosis, rheumatoid arthritis, or psoriasis. In a particular embodiment thereof, the mammalian subject is a human.

Also encompassed herein is a method for treating a mammalian subject afflicted with a cancer, the method comprising administering an enhancer of DDX5 polypeptide activity identified using methods described herein to the subject, wherein the enhancer of DDX5 polypeptide activity increases T_{H}17 cell activity in the mammalian subject and thereby treats the subject. In an embodiment thereof, the mammalian subject is afflicted with gastric adenocarcinoma, lung cancer, ovarian cancer, melanoma, glioblastoma, or pancreatic cancer. In a particular embodiment thereof, the mammalian subject is a human.

In a further aspect, a method of treating a mammalian subject afflicted with an inflammatory condition or autoimmune disease associated with a T_{H}17 cell mediated pathology is presented, the method comprising administering an inhibitor of DDX5 polypeptide activity to the subject, wherein the inhibitor of DDX5 polypeptide activity reduces T_{H}17 cell activity in the subject and thereby treats the mammalian subject, wherein the inhibitor in vitro or in vivo decreases RORγt or RORγt fragment/DDX5/Rmrp complex formation in the presence of the inhibitor as compared to RORγt or RORγt fragment/DDX5/Rmrp complex formation in the absence of the inhibitor.

In a still further aspect, a method of treating a mammalian subject afflicted with an inflammatory condition or autoimmune disease associated with a T_{H}17 cell mediated pathology is presented, the method comprising administering an inhibitor of DDX5 polypeptide activity to the subject, wherein the inhibitor of DDX5 polypeptide activity reduces T_{H}17 cell activity in the subject and thereby treats the mammalian subject, wherein the inhibitor in vitro or in vivo decreases DDX5/Rmrp complex formation in the presence of the inhibitor as compared to DDX5/Rmrp complex formation in the absence of the inhibitor.

Also encompassed herein is an in vivo method of decreasing RORγt or RORγt fragment/DDX5/Rmrp complex formation, the method comprising administering an inhibitor of DDX5 polypeptide activity to a subject, wherein the inhibitor of DDX5 polypeptide activity reduces T_{H}17 cell activity in the subject, wherein the inhibitor in vitro or in vivo decreases RORγt or RORγt fragment/DDX5/Rmrp complex formation in the presence of the inhibitor as compared to RORγt or RORγt fragment/DDX5/Rmrp complex formation in the absence of the inhibitor.

In a further embodiment, an in vivo method of decreasing DDX5/Rmrp complex formation is presented, the method comprising administering an inhibitor of DDX5 polypeptide activity to a subject, wherein the inhibitor of DDX5 polypeptide activity reduces T_{H}17 cell activity in the subject, wherein the inhibitor in vitro or in vivo decreases DDX5/Rmrp complex formation in the presence of the inhibitor as compared to DDX5/Rmrp complex formation in the absence of the inhibitor.

Also envisioned herein is a method for treating a mammalian subject afflicted with a cancer, the method comprising administering an enhancer of DDX5 polypeptide activity to the subject, wherein the enhancer of DDX5 polypeptide activity increases T_{H}17 cell activity in the mammalian subject and thereby treats the subject, wherein the inhibitor in vitro or in vivo enhances DDX5/Rmrp complex formation in the presence of the enhancer as compared to DDX5/Rmrp complex formation in the absence of the enhancer.

In a further aspect, a method for treating a mammalian subject afflicted with a cancer is presented, the method comprising administering an enhancer of DDX5 polypeptide activity to the subject, wherein the enhancer of DDX5 polypeptide activity increases T_{H}17 cell activity in the mammalian subject and thereby treats the subject, wherein the inhibitor in vitro or in vivo enhances RORγt or RORγt fragment/DDX5/Rmrp complex formation in the presence of the enhancer as compared to RORγt or RORγt fragment/DDX5/Rmrp complex formation in the absence of the enhancer.

In another embodiment, an in vivo method of increasing or enhancing RORγt or RORγt fragment/DDX5/Rmrp complex formation is presented, the method comprising administering an enhancer of DDX5 polypeptide activity to a subject, wherein the enhancer of DDX5 polypeptide activity increases T_{H}17 cell activity in the subject, wherein the enhancer in vitro or in vivo increases or enhances RORγt or RORγt fragment/DDX5/Rmrp complex formation in the presence of the enhancer as compared to RORγt or RORγt fragment/DDX5/Rmrp complex formation in the absence of the enhancer.

An in vivo method of increasing or enhancing DDX5/Rmrp complex formation is also envisioned, the method comprising administering an enhancer of DDX5 polypeptide activity to a subject, wherein the enhancer of DDX5 polypeptide activity increases T_{H}17 cell activity in the subject, wherein the inhibitor in vitro or in vivo increases or enhances DDX5/Rmrp complex formation in the presence of the enhancer as compared to DDX5/Rmrp complex formation in the absence of the enhancer.

Other features and advantages of the invention will be apparent from the following description of the particular embodiments thereof, and from the claims.

### BRIEF DESCRIPTION OF THE DRAWINGS

FIGS. 1A-E. Requirement for DDX5 in T_{H}17 cytokine production in vitro and at steady state in vivo. A, Selective T_{H}17 cell differentiation defect in DDX5-deficient T cells (DDX5-T) after polarization for 96 h. Representative of three independent experiments. WT, wild type. B, Volcano plot of RNA-seq of cultured T_{H}17 cells from DDX5-T mice and littermate controls. Black dots, differentially expressed genes (minimum fold change of two with P<0.05). Blue dots, known RORγt-dependent genes. Red dots, top RORγt−DDX5-coregulated genes. C, D, SFB colonization and percentage and number of RORγt+CD4+ T cells (C) and number of IL-17A-producing CD4+ T cells (D) in ileal lamina propria of co-housed wild-type (+/+; n=5) and DDX5-T (fl/fl; n=5) CD4-Cre+ mice. Graphs show mean±s.d. from two independent experiments, combined. NS, not significant. **P<0.01 (paired t-test). E, Representative IL-17A expression in CD4+Foxp3−RORγt+T_{H}17 cells from ileal lamina propria of wild-type and DDX5-T mice after restimulation.

FIGS. 2A-E. Role of DDX5 in mouse models of T_{H}17-cell-mediated autoimmune disease. A, Weight change in Rag2−/− recipients of wild-type or DDX5-T CD4+ naive T cells in the transfer model of colitis measured on days 0, 10, 25, 37 and 45 (PBS, n=4; wild type, n=9; DDX5-T, n=13, combined from three independent experiments). i.p., intraperitoneal. B, Haematoxylin and eosin (H&E) staining and analysis of large intestine at day 45. Representative sections (scale bars, 100 μm) and histology scores (scale of 0-24) are shown. Scores for PBS (n=3), wild-type (red, n=8) and DDX5-T (blue, n=7) mice are from two independent experiments. C, Cytokine production defect in DDX5-T T_{H}17 (RORγt+) but not T_{H}1 (RORγt−T-bet+) cells in large intestine lamina propria at day 45 (n=4 per group). D, EAE disease scores (scale of 0-5) in co-housed myelin oligodendrocyte glycoprotein (MOG)-immunized littermates. Wild-type (n=13) and DDX5-T (n=11) mice, combined from three independent experiments. E, Defective IL-17A production in DDX5-T CD4+RORγt+ cells in the spinal cord of MOG-immunized mice (n=7 per group). Graphs show mean±s.d. *P<0.05, **P<0.01; ***P<0.001 (unpaired, t-test).

FIGS. 3A-E. Requirement for helicase-competent DDX5 and its associated lncRNA Rmrp in induction of T_{H}17 cell cytokines. A, B, Cytokine production in DDX5-T cells transduced with wild-type or helicase-mutant DDX5 and subjected to sub-optimal T_{H}17 cell polarization. Results from four independent experiments shown. C, IGV browser view of Rmrp showing coverage of mapped RNA reads from total T_{H}17 lysate, ribosome TRAP-seq (EGFP-L10; described in Methods), DDX5 RIP-seq and RORγt RIP-seq. IP, immunoprecipitate. D, Effect of mouse Rmrp-specific ASO. Results are representative of three independent experiments with two technical replicates. Ctrl, control. E, IL-17A production following RMRPknockdown in in vitro polarized human T_{H}17 cells. Each data point (right panel) represents cells from a healthy donor (n=5). Graphs show mean±s.d. **P<0.01; ****P<0.0001 (unpaired, t-test).

FIGS. 4A-F. Analysis of DDX5-dependent Rmrp function in T_{H}17 cell differentiation. A, B, Cytokines in wild-type and DDX5-T in vitro polarized T_{H}17 cells after Rmrp knockdown (A) or overexpression (B). Representative of three independent experiments. C, Sequence of the Rmrp gene (nucleotides 258-275) from wild-type and RmrpG270T/G270Tlittermates. D, Rmrp-dependent expression of a RORE-directed firefly luciferase reporter nucleofected into polarized T_{H}17 cells at 72 h. Firefly and control Renilla luciferase activities were measured 24 h later. Each dot represents the result from one nucleofection. Results from two independent experiments. DMSO, dimethyl sulfoxide; RLU, relative luciferase units; RORγinh, RORγ antagonist ML209. E, Top RORγt targets coregulated by DDX5 and Rmrp. F, Proportion of CD4+Foxp3−T cells expressing RORγt (left) and numbers of TH1 (IFNγ+RORγt−Tbet+), T_{H}17 (IL-17A+RORγt+Foxp3−) and Tγδ17 (IL-17A+Tγδ+ RORγt+) cells (right) in small intestine lamina propria. Symbols represent cells from one mouse. Graphs show mean±s.d. **P<0.01; ***P<0.001; ****P<0.0001 (unpaired, t-test).

FIGS. 5A-E. Rmrp localization at RORγt-occupied genes and role in RORγt-DDX5 assembly. A, RORγt association with immunoprecipitated DDX5 in polarized T_{H}17 cells. IB, immunoblot. Representative of three independent experiments. B, Rmrp quantification by RT-qPCR in RORγt immunoprecipitates from polarized T_{H}17 cells. Representative of two independent experiments with two technical replicates. C, Rmrp requirement for ATP-dependent in vitro interaction of recombinant (r) glutathione S-transferase (GST)-DDX5 and His-RORγt. Representative of three independent experiments. IVT, in vitro transcribed RNA. For uncropped gels (A, C), see FIG. 6. D, Rmrp occupancy at RORγt genomic target loci in polarized T_{H}17 cells. Rmrp ChIRP-qPCR amplicons (bottom) are indicated in IGV browser view of RORγt ChIPseq at the Il17 locus (top). Data from 2-4 experiments with two technical replicates. E, Model for DDX5-Rmrp complex recruitment to RORγt occupied chromatin in T_{H}17 cells. Graphs show mean±s.d. **P<0.01 (unpaired, t-test).

FIGS. 6A-F. Identification of DDX5 as a RORγt interacting partner. A, Mass spectrometry experimental workflow. Sorted naive CD4+ T cells from wild-type mice were cultured in vitro in T_{H}17-polarizing conditions for 48 h. Immunoprecipitation of endogenous RORγt was performed using RORγ/γt-specific antibodies on whole-cell lysates. RORγt enrichment in pull-down was confirmed by immunoblot. Immunoprecipitated proteins were digested and analysed by mass spectrometry. The listed DDX5 peptides were identified in the T_{H}17 RORγt immunoprecipitate. B, Co-immunoprecipitaton of DDX5 with anti-RORγt in lysates of in vitro polarized T_{H}17 cells. C, Cell surface phenotype of splenic and lymph node DAPI−CD4+CD8α−CD19− T cells from wild-type and DDX5-T mice, examined by flow cytometry. D, Immunoblot of RORγt protein expression whole-cell lysate of cultured T_{H}17 cells from wild-type or DDX5-T animals. For uncropped gels (B, D), see FIG. 6. E, Immunofluorescence staining of RORγt in cultured T_{H}17 cells from wild-type or DDX5-T mice. F, Immunofluorescence staining revealed nuclear localization of DDX5 in T_{H}17 cells.

FIGS. 7A-D. DDX5 coregulates a subset of RORγt transcriptional targets in polarized T_{H}17 cells. A, Venn diagram of distinct and overlapping genes regulated by RORγt and/or DDX5, as determined from RNA-seq studies. B, Ingenuity Pathway Analysis (Qiagen) of DDX5- and RORγt-coregulated genes. C, IGV browser view showing biological replicate RNA-seq coverage tracks of control and DDX5-T from in vitro polarized T_{H}17 cell samples at the Il17a, Il22, Ddx5 and Rorc loci. D, Independent RT-qPCR validation of RNA-seq results confirming effects of DDX5 deletion on RORγt target gene expression. Graphs show mean±s.d.

FIGS. 8A-C. DDX5 chromatin localization in T_{H}17 cells. A, ChIP-seq-generated heatmap of DDX5 occupancy in regions centered on 16,003 RORγt-occupied sites (±2 kilobases (kb)). K-means linear normalization was used for clustering analysis by SeqMiner. Metagene analysis on cluster 1 depicts RORγt-occupied regions with DDX5 enrichment in wild-type but not DDX5-T cells; cluster 2 represents RORγt-occupied regions without DDX5 enrichment. B, IGV browser view of Il17f and Rorc loci with ChIP-seq enrichment for RNA Pol II, RORγt and DDX5. C, Independent ChIP-qPCR of DDX5 in polarized T_{H}17 cells. DDX5 occupancy at the Il17a and Il17f loci (as identified by RORγt ChIP-seq MACS peak called 32 and 39, respectively, from B) in control, DDX5-T or RORγt-deficient (RORγKO) cells. Results are representative of two independent experiments. Each experiment was performed with two technical replicates. Graph shows mean±s.d. **P<0.01 (unpaired, t-test).

FIGS. 9A-C. Influence of DDX5 on T-cell phenotypes in autoimmune disease models. A, At 8 weeks after T-cell transfer, large intestine lamina propria mononuclear cells were evaluated for amounts of Il17a and lfng mRNA by RT-qPCR. Results are representative of two independent experiments. Each experiment was performed using large intestines from three mice in each condition. RT-qPCR was performed with two technical replicates. Graph shows mean±s.d. *P<0.03 (unpaired, t-test). B, Gating strategy for analysis of T_{H}17 and T_{H}1 cells from large intestine of Rag2-deficient recipients of wild-type or DDX5-T naive T cells analysed at 8 weeks after T-cell transfer. C, Representative IL-17A and IFNγ intracellular staining of Aqua⁻CD4⁺RORγt⁺ T_{H}17 cells in spinal cord of MOG-immunized animals on day 21.

FIGS. 10A-D. Noncoding RNAs enriched in DDX5 and RORγt RIP-seq studies. A, DDX5-T cells were transduced with wild-type or helicase-mutant DDX5 and evaluated for DDX5 expression by immunofluorescence (left) and immunoblot (right) with anti-DDX5 antibody. For uncropped gels, see FIG. 6. B, Venn diagram of noncoding RNAs detected by RIP-seq of ribosome-depleted T_{H}17 cell lysates with anti-DDX5 and anti-RORγt antibodies. C, Abundance of top noncoding RNAs enriched in DDX5 and RORγt immunoprecipitates from polarized T_{H}17 cell lysates depleted of ribosomes. Top, abundance of the noncoding RNAs in total lysate. D, RIP-qPCR experiments to compare Rmrp association with DDX5 in cultured T_{H}17 and total thymocytes ex vivo. Results are representative of three independent experiments. Each experiment was performed with two technical replicates. Graph shows mean±s.d. **P<0.001 (unpaired, t-test).

FIGS. 11A-C. Rmrp and DDX5 knockdown in mouse and human T_{H}17 cells. A, RNA FISH analysis, using probes specific for Rmrp (green) and Malat1 (red) lncRNAs, in T_{H}17 cells at 72 h after nucleofection with control (CTL) or Rmrp ASOs. B, Effect of Rmrp ASOs targeting different regions of Rmrp transcript on levels of Rmrp, Il17f and Ccr6 RNAs in polarized T_{H}17 cells. C, Knockdown of DDX5 reduced IL-17A production in in vitro polarized human RORγt⁻T_{H}17 cells. **P<0.01 (paired, t-test). Representative result shown in left panel. Each dot represents a different healthy donor (n=4). Graphs show mean±s.d.

FIGS. 12A-G. Effects of wild-type and mutant Rmrp in T-cell differentiation. A, Il17a mRNA in cell lysates of in vitro polarized mouse T_{H}17 cells at 96 h after transduction of control vector or wild-type Rmrp. Results are representative of two independent experiments. B, IFNγ production in polarized mouse T_{H}1 cells at 96 h after transduction of control or Rmrp-encoding vector. Representative of two independent experiments. Each experiment was performed with two technical replicates. C, Comparison of human and mouse Rmrp sequences. Several mutations identified in CHH patients are highlighted. D, IL-17A production in polarized mouse T_{H}17 cells at 96 h after transduction of wild-type or mutant Rmrp vectors. Representative of two independent experiments. E, Venn diagram depicting the number of distinct and overlapping genes regulated by RORγt, DDX5 and Rmrp in in vitro polarized T_{H}17 cells. F, Expression of cytokine and Foxp3 mRNAs in T cells from wild-type or Rmrp^{G270T/G270T} mice cultured in vitro in T_{H}17-, iT_{reg}-, T_{H}1- and T_{H}2-polarizing conditions. Results are representative of two independent experiments. Each experiment was performed with two technical replicates. ***P<0.001 (unpaired, t-test). g, ChIP-qPCR experiment using anti-RORγ/γt antibodies on chromatin of T_{H}17 cells from wild-type or mutant mice cultured for 48 h in vitro. Each dot represents a different biological sample. Wild type, n=2; Rmrp^{G270T}, n=2. Results are representative of three separate independent experiments. Graphs show mean±s.d. (unpaired, t-test).

FIGS. 13A-C. Effect of Ddx5 and Rmrp mutations in inflammation and thymocyte development. A, Left, percentage weight change in Rag2^{-/-} recipients of wild-type (black circles) or Rmrp^{G270T/G270T} (grey squares) naive CD4⁺ T cells in the transfer model of colitis. Animal weight was measured on day 56 (wild type, n=8; Rmrp^{G270T/G270T}, n=8, combined from three independent experiments). Graphs show mean±s.d. ***P<0.001 (unpaired, t-test). Middle, histology score (scale of 0-24) (wild type, n=8; Rmrp^{G270T/G270T}, n=5), combined from two independent experiments. **P<0.01 (unpaired, t-test). Right, representative H&E staining of large intestine from Rag2^{-/-} mice on day 56 after naive T-cell transfer. B, Mice with deletion of Ddx5 in early common lymphoid progenitors (clp) have normal thymic development. Left, immunoblot of thymocyte lysates with anti-DDX5 antibody confirmed depletion of DDX5. Right, percentage of CD4 single-positive (SP), CD8α SP, double-positive (DP) and double-negative (DN) cells among total thymocytes. Each bar represents the result from one mouse (WT/het, n=9; DDX5-clpKO, n=6). For uncropped gels, see FIG. 6. C, Thymocyte and peripheral T-cell surface phenotypes of wild-type and Rmrp^{G270T/G270T} knock-in mice at steady state. Peripheral T-cell gate, DAPI⁻CD19⁻CD8α⁻CD4⁺.

FIGS. 14A-D. Association of Rmrp lncRNA with DDX5 and RORγt in vitro. A, In vitro translated (TNT) HA-tagged wild-type or helicase-dead DDX5 and Flag-tagged RORγt were incubated with in vitro transcribed Rmrp. After capture on anti-HA or anti-Flag beads, the amount of lncRNA was determined by RT-qPCR. Data are representative of two independent experiments, and each experiment was performed with two technical replicates. B, Helicase requirement for in vitro interaction of DDX5 and RORγt. Recombinant GST-DDX5 (wild type or helicase-dead mutant) and His-RORγt full-length protein were synthesized in Escherichia coli, purified, and assayed for binding with or without in vitro transcribed Rmrp RNA in the presence exogenous ATP. C, Association of in vitro transcribed wild-type and mutant Rmrp with recombinant GST-DDX5 captured on glutathione beads (left) or with recombinant GST-DDX5 and His-RORγt captured with anti-His antibody. Amounts of associated Rmrp were quantified using RT-qPCR. Data are representative of two independent experiments. Each experiment was performed with two technical replicates. D, Comparison of ability of in vitro transcribed wild-type and Rmrp^{G270T} lncRNA to promote interaction between recombinant RORγt and DDX5 in vitro. All graphs show mean±s.d. ***P<0.001 (unpaired, t-test). For uncropped gels, see FIG. 6.

FIGS. 15A-F. Rmrp chromatin localization in T_{H}17 cells. A, ChIRP-seq sample validation of Rmrp RNA pull-down over other nuclear noncoding RNAs using pools of ‘even’ or ‘odd’ capture probes. Graphs show mean±s.d. B, ChIRP-qPCR of Rmrp RNA pull-down from wild-type T_{H}17 cell lysate treated with or without RNase (n=2). qPCR for each sample was performed with two technical replicates. Graph shows mean±s.d. **P<0.001 (unpaired, t-test). C, HOMER motif analysis reveals top three DNA motifs within Rmrp-enriched peaks. D, Significance of peak overlaps between Rmrp ChIRP-seq and ChIP-seq for BATF (n=2), IRF4 (n =7), STAT3 (n=2), c-Maf (n=2), RORγt (n=2), CTCF (n=2), RNA Pol II (n=2), H3K27me3 (n=4) and H3K4me3 (n=3) in T_{H}17 cells (hypergeometric distribution). Each dot represents a separate biological replicate of ChIP-seq experiments. E, Venn diagram depicting changes in peaks called from Rmrp (ChIRP-seq) experiments in wild-type and DDX5-T T_{H}17 cells. F, Comparison of Rmrp chromatin occupancy (ChIRP-seq) at known RORγt occupied loci in in vitro polarized T_{H}17 cells from wild-type and Rmrp^{G27OT/G270T} mice.

FIGS. 16A-B. Histogram plots showing in vitro transcribed wildtype and CHH patient mutation carrying Rmrp RNA transcripts exhibit differential binding affinities for recombinant GST-DDX5 and His-RORgt proteins in pull-down assays.

FIG. 17. Depicts a SHAPE study performed using cultured wild type mouse T_{H}17 cells. Top: icSHAPEseq data on the Rmrp transcript identifies candidate nucleotides of Rmrp that may provide exposed contact surfaces for binding to protein partners such as DDX5. Bottom: Cas9-guide RNA directed cleavage at these sites generate indel mutations, resulting in reduced T_{H}17 cytokine production in cultured mouse T_{H}17 cells.

FIG. 18. Amino acid sequences of human nuclear receptor ROR-gamma isoforms a and b and mouse nuclear receptor ROR-gamma isoform 2 and an alignment thereof. In each of the ROR-gamma isoforms, the ligand binding domain starts at the amino acid sequence “YGSPSFRSTPEAP” (SEQ ID NO: 13) and continues through to the end of the protein; the precise position of which domain depends on the isoform.

FIG. 19. Amino acid sequences of human DDX5 and mouse DDX5 and an alignment thereof.

FIGS. 20A-B. A) Alignment of nucleic acid sequences of human Rmrp and mouse Rmrp and B) cartoon depicting mutations detected in Rmrp.

FIGS. 21A-E. Exemplary nucleic acid sequences encoding human nuclear receptor ROR-gamma isoforms a (A; SEQ ID NO: 61) and b (B; SEQ ID NO: 62) and mouse nuclear receptor ROR-gamma isoform 2 (C; SEQ ID NO: 63) and exemplary nucleic acid sequences encoding human (D; SEQ ID NO: 64) and mouse (E; SEQ ID NO: 65) DDX5.

### DETAILED DESCRIPTION OF THE INVENTION

T helper 17 (T_{H}17) lymphocytes protect mucosal barriers from infections, but also contribute to multiple chronic inflammatory diseases. Their differentiation is controlled by RORγt, a ligand-regulated nuclear receptor. The present inventors herein identify the RNA helicase DEAD-box protein 5 (DDX5) as a RORγt partner that coordinates transcription of selective T_{H}17 genes, and is required for T_{H}17-mediated inflammatory pathologies. Surprisingly, the ability of DDX5 to interact with RORγt and coactivate its targets depends on intrinsic RNA helicase activity and binding of a conserved nuclear long noncoding RNA (lncRNA), Rmrp, which is mutated in patients with cartilage-hair hypoplasia. A targeted Rmrp gene mutation in mice, corresponding to a gene mutation in cartilage-hair hypoplasia patients, altered lncRNA chromatin occupancy, and reduced the DDX5−RORγt interaction and RORγt target gene transcription. Elucidation of the link between Rmrp and the DDX5−RORγt complex reveals a role for RNA helicases and lncRNAs in tissue-specific transcriptional regulation, and provides new opportunities for therapeutic intervention in T_{H}17-dependent diseases. See also Huang et al. (2015, Nature 528:517-522), the entire content of which is incorporated herein by reference.

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

The term “complementary” refers to two DNA strands that exhibit substantial normal base pairing characteristics. Complementary DNA may, however, contain one or more mismatches.

The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” or “purified nucleic acid” is sometimes used. This term, when applied to DNA, refers to a DNA molecule that is separated from sequences with which it is immediately contiguous in the naturally occurring genome of the organism in which it originated. For example, an “isolated nucleic acid” or “purified nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” or “purified nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated or purified nucleic acid (either DNA or RNA) may further represent a molecule produced directly by biological or synthetic means and separated from other components present during its production.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the term “operably linked” refers to a regulatory sequence capable of mediating the expression of a coding sequence and which is placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4, 7, 2′, 7′-Tetrachloro-6-carboxyfluorescein (TET). Other alternative DNA labeling methods are known in the art and are contemplated to be within the scope of the invention.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More particularly, the preparation comprises at least 75% by weight, and most particularly 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, “transduce”, shall refer to any method or means by which a nucleic acid is introduced into a cell or host organism and may be used interchangeably to convey the same meaning. Such methods include, but are not limited to, viral transduction, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other applications, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

An “immune response” signifies any reaction produced by an antigen, such as a protein antigen, in a host having a functioning immune system. Immune responses may be either humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems.

An “antibody” or “antibody molecule” is any immunoglobulin, including antibodies and fragments thereof, that binds to a specific antigen. The term includes polyclonal, monoclonal, chimeric, and bispecific antibodies. As is understood in the art, such antibodies can be of any mammalian species, including, without limitation: humans, other primates, mouse, rat, or a camelids. As used herein, antibody or antibody molecule contemplates both an intact immunoglobulin molecule and an immunologically active portion of an immunloglobulin molecule such as those portions known in the art as Fab, Fab′, F(ab′)2 and F(v).

The term “about” as used herein refers to a variation in a stated value or indicated amount of up to 10%, 9%, 8%, 7%, 6%, 5%, 4%, 3%, 2%, 1%, 0.75%, 0.5%, 0.25% or 0.1%., wherein the variation can be either an increase or a decrease in the stated value or indicated amount. Use of the term may, therefore, be used to establish a range of values or amounts.

As used herein, the term “modulator” refers to a compound or molecule that is capable of altering an activity such that the activity is either inhibited/decreased or enhanced/increased in the presence of the modulator relative to the level of activity in the absence of the modulator or in the presence of a negative control compound. Modulators can, therefore, either be inhibitors or enhancers of the activity being measured. With respect to the present screening methods, the presence of an inhibitor of Rmrp, for example, decreases or inhibits Rmrp activity, whereas an enhancer of Rmrp increases or enhances Rmrp.

As used herein, the term “candidate compound” or “test compound” refers to any compound or molecule that is to be tested. As used herein, the terms, which are used interchangeably, refer to biological or chemical compounds such as simple or complex organic or inorganic molecules, peptides, proteins, peptidomimetics, peptide mimics, antibodies, nucleic acids (DNA or RNA), oligonucleotides, polynucleotides, antisense molecules (e.g., anti-sense oligonucleotides), small interfering nucleic acid molecules, including siRNA or shRNA, carbohydrates, lipoproteins, lipids, small molecules and other drugs. In a particular embodiment, an anti-sense oligonucleotide targets Rmrp. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed.

As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action. Moreover, the agent may be selected by its effect on the gene expression profile obtained from screening in vitro or in vivo. Furthermore, candidate compounds can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

If the screening for compounds that modulate the expression, activity or function of RORγt polypeptide activity is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled artisan. In addition, a secondary screen may include assessing the effect of a candidate compound on the release of inflammatory cytokines such as, for example, IL17A, IL17F, and/or IL22 using standard procedures known in the art.

### Screening/Testing for Modulators of Th 17 Cell Activity

Any screening technique known in the art can be used to screen for candidate compounds that modulate T_{H}17 cell, RORγt, DDX5, and/or Rmrp activity/function. Such candidate compounds may, for example, modulate formation of complexes comprising at least two of RORγt, DDX5, and Rmrp or functional fragments of any one of these complex components. Exemplary complexes comprise RORγt, DDX5, and Rmrp or functional fragments thereof; DDX5 and Rmrp or functional fragments thereof; and RORγt and DDX5 or functional fragments. The present invention contemplates screens for small molecule modulators, as well as screens for natural proteins or peptides that bind to and modulate RORγt, DDX5, and/or Rmrp activity or function. For example, natural products or peptide libraries can be screened using assays described herein to identify molecules that have the ability to modulate RORγt, DDX5, and/or Rmrp activity and/or T_{H}17 cell activity, e.g., to inhibit T_{H}17 mediated inflammatory reactions.

Screening assays described herein that comprise RORγt, DDX5, and Rmrp may be used to identify, for example, an inhibitor of DDX5 helicase activity, a competitive binding molecule to block Rmrp binding, and/or a molecule that alters the structure of Rmrp so that it no longer interacts with DDX5, cannot be structurally converted by helicase, or cannot bind to RORγt despite conversion by helicase.

Exemplary assays envisioned herein include in vitro binding assays, wherein, e.g., full length His-tag RORγt and GST-tag DDX5 are present in a polypeptide mixture comprising Rmrp and, e.g., 25mM Tris, 100 mM NaCl, 0.5% NP40, 10 mM MgCl₂, 10% glycerol, ATP and protease inhibitors. See, for example, Draper (2004, RNA 10:335-343; the entire content of which is incorporated herein by reference). In one embodiment, RORγt is immobilized on an anti-His column. DDX5 binding may, e.g., be detected by SDS-PAGE Western blot analysis using anti-GST antibodies. Rmrp RNA binding thereto may be detected after purification of bound RNA molecules by Trizol extraction, followed by reverse transcription-cDNA synthesis and quantitative PCR (qPCR).

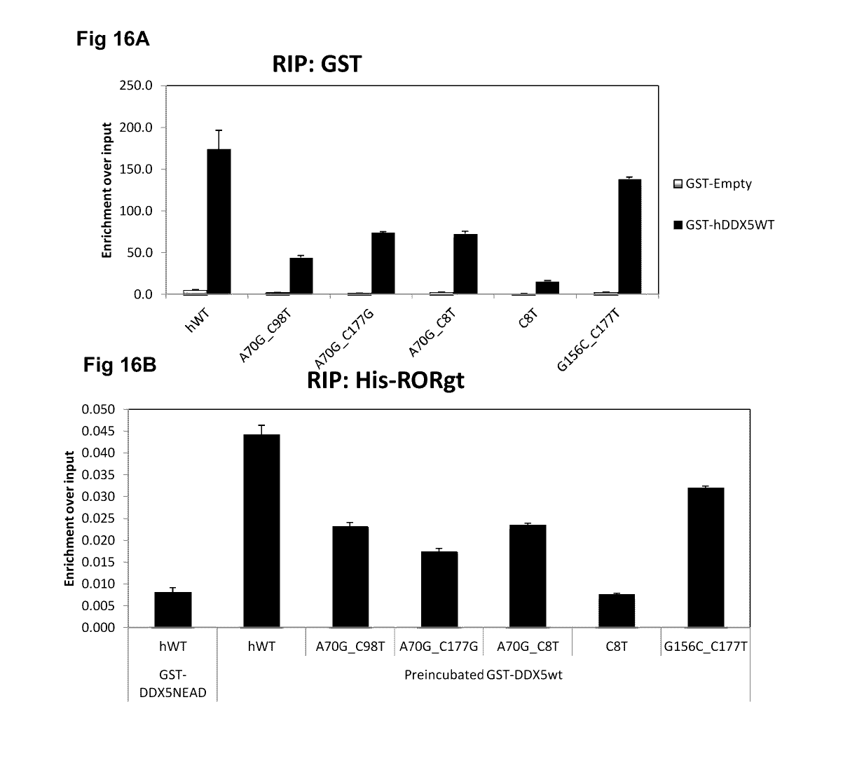

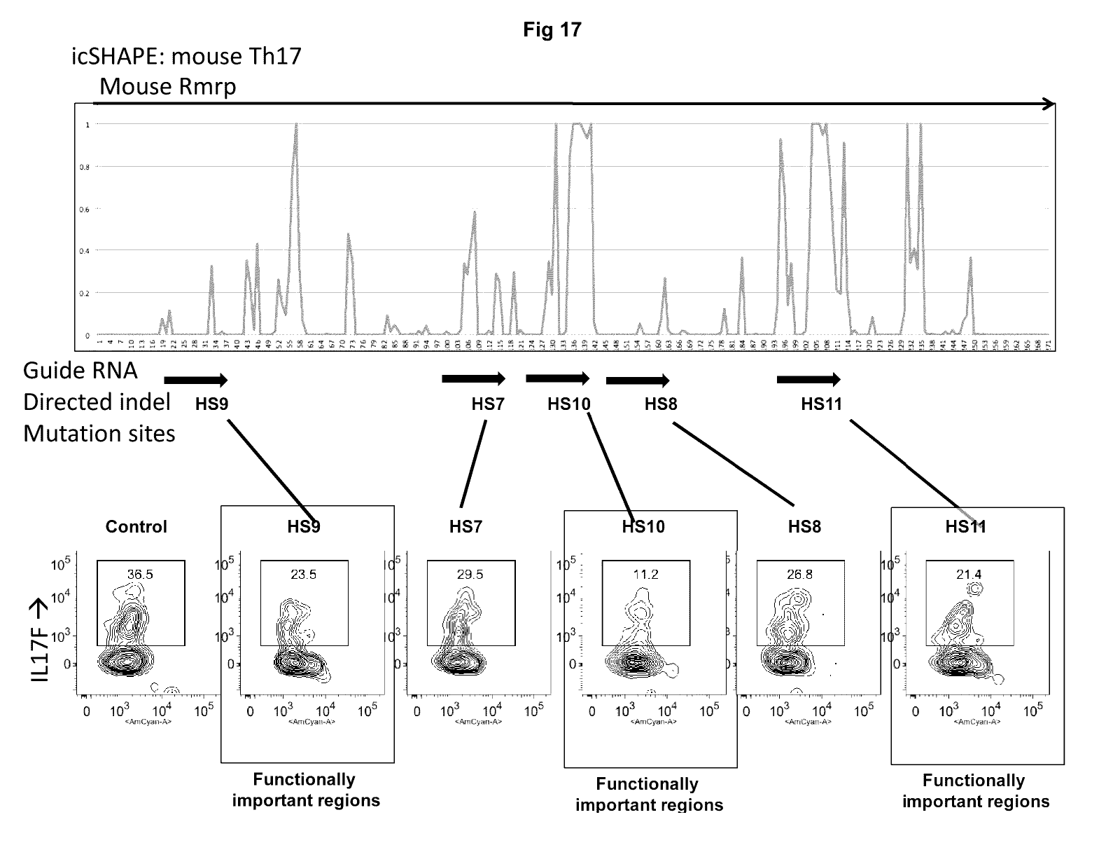

Further to the above, the present inventors have performed screens for human Cartilage Hair Hypoplasia patient mutations on the non-coding RNA Rmrp that change binding to the RNA helicase DDX5 and/or RORγt. See, for example, FIG. 16. These results demonstrate that human Rmrp RNA transcripts comprising A70G_C98T, A700G_C177G, A70G_C8T, and C8T mutations exhibit less binding to DDX5 and RORγt proteins as compared to wildtype Rmrp RNA in vitro.

Alternatively, high-throughput Surface Plasmon Resonance (GE) may be used for a quick readout of protein-protein-RNA complex interaction changes. Protein-protein interactions may also be assayed using FRET or alphascreen or other protein interaction assays in high throughput format, with or without Rmrp. Rmrp would promote interaction, thus facilitating screens for inhibitors. Such assays may also be used to screen for Rmrp mimics that enhance RORγt and DDX5 interaction.

In another embodiment, the aforementioned in vitro binding assay may be performed by utilizing in vitro SHAPEseq analysis to determine changes in Rmrp secondary structures in the presence of candidate modulators.

Further to the above, the present inventors sought to determine experimentally the secondary structure of RMRP and to this end, performed SHAPE (selective 2′-hydroxyl acylation analyzed by primer extension) studies. The SHAPE reagent chemically acrylates 2′-ribose hydroxyl groups of RNA nucleotides that are conformationally flexible, or unconstrained by intra- or inter-molecular interactions. Reverse transcription (RT) of modified RNAs is blocked at the nucleotides with the modification, resulting in a pool of cDNAs whose length distribution reflects the distribution of modifications across each RNA. Next generation deep sequencing revealed peaks of highly reactive and flexible nucleotides and valleys of lowly reactive nucelotides that may be involved in base pairing/rigid stem structures. Wildtype mouse T_{H}17 cells were cultured in vitro in the presence of an in vivo icSHAPE reagent (see, for example, Mortimer et al. 2012, Current Protocols in Chemical Biology 4:275-297; the entire content of which is incorporated herein by reference). Endogenous RNAs were modified and Rmrp specific primers were used in primer extension experiments to reveal Rmrp secondary structures in T_{H}17 cells. These results show that nucleotides 55-58, 130-132, 134-140, 194-196, 204-210, 213-214, 230-236 of Rmrp are conformationally flexible, or unconstrained by intra- or inter-molecular interactions in cultured mouse T_{H}17 cells. Introduction of guide RNAs into Cas9 expressing T_{H}17 cells (see, for example, Platt et al. 2014, Cell 159:440-455; the entire content of which is incorporated herein by reference) generates indel mutations at the indicated target sites. HS9, HS10, and HS11 guide RNA directed indel mutations reduced IL17F cytokine production as indicated by intracellular staining and flow cytometry.

In yet another embodiment, in vitro transcription assays may be implemented to determine changes in RORγt transcriptional activity in vitro in the presence of the different molecules and the candidate modulators. Briefly, RORγt (+DDX5+Rmrp+/-modulators) is incubated in the presence of total rabbit retinulocyte lysate (which comprises mammalian RNA polymerase II and other core transcriptional machinery) along with a DNA plasmid vector that encodes a reporter gene (e.g., a Firefly-Luciferase gene) under the control of ROR-responsive element along with a core minimal promoter. Measuring RORγt driven luciferase activity provides a direct transcriptional activity read out of the complex in vitro.

For assays in primary cells, sorted naive CD4 T cells may be isolated from mouse spleen or human peripheral blood that are polarized under T_{H}17 promoting conditions in vitro. Candidate soluble small molecules may be added to the culture media directly and evaluated for their ability to modulate T_{H}17 cell activity. Non-permeable/large candidate molecules may be introduced into the cells by, e.g., electroporation. IL17A, IL17F, and/or IL22 expression may be used as a readout to determine if a candidate soluble small molecule or non-permeable/large candidate molecule can act as a modulator of T_{H}17 cell activity. In a particular embodiment thereof, exogenous DDX5 and Rmrp are transduced into the polarized T_{H}17 cells so as to generate a “super-activated” state, which facilitates the identification of inhibitors of cytokine expression.

For higher throughput assays, a human 293 ft fibroblast cell line, e.g., that stably comprises a reporter gene (e.g., a Firefly-Luciferase gene) under the control of ROR-responsive element along with a core minimum promoter may be generated. Candidate soluble small molecule modulators may be added to the culture media directly. Non-permeable/large candidate modulator molecules may be introduced into the cells by electroporation. RORγt driven luciferase activity may be measured to provide a transcriptional activity readout at the end of 24-48 hour of culture in the presence/absence of candidate modulators. Cell based assays with stable high expression of exogenous DDX5 and RORγt may, for example, be used to find enhancers of association/transcriptional activity and inhibitors if Rmrp is also expressed in the cells. Many variations of such cell based assays are envisioned herein, including use of Rmrp mutant cells to have minimal background activity. Rmrp mutant cells may be generated via, for example, clustered regularly interspaced short palindromic repeats-Cas (CRISPR-Cas) technology.

Identification and screening of a molecule is further facilitated by determining structural features of the protein, e.g., using X-ray crystallography, neutron diffraction, nuclear magnetic resonance spectrometry, and other techniques for structural assessment and determination. These techniques provide for the rational design or identification of proteins, peptide fragments, or small molecules that have a modulatory effect on RORγt, DDX5, and/or Rmrp activity or function.

Another approach uses recombinant bacteriophage to produce large libraries. Using the “phage method” [Scott and Smith, 1990, Science 249:386-390 (1990); Cwirla, et al., Proc. Natl. Acad. Sci., 87:6378-6382 (1990); Devlin et al., Science, 249:404-406 (1990)], very large libraries can be constructed (10⁶-10⁸ chemical entities). A second approach uses primarily chemical methods, of which the Geysen method [Geysen et al., Molecular Immunology 23:709-715 (1986); Geysen et al. J. Immunologic Method 102:259-274 (1987)] and the method of Fodor et al. [Science 251:767-773 (1991)] are examples. Furka et al. [14th International Congress of Biochemistry, Volume 5, Abstract FR:013 (1988); Furka, Int. J. Peptide Protein Res. 37:487-493 (1991)], Houghton [U.S. Pat. No. 4,631,211] and Rutter et al. [U.S. Pat. No. 5,010,175] describe methods to produce a mixture of peptides that can be tested as activators or inhibitors.

Screening phage-displayed random peptide libraries offers a rich source of molecular diversity and represents a powerful means of identifying peptide ligands that bind a receptor molecule of interest (Cwirla, et al., Proc. Natl. Acad. Sci., 87:6378-6382 (1990); Devlin et al., Science, 249:404-406 (1990)). Phage expressing binding peptides are selected by affinity purification with the target of interest. This sytem allows a large number of phage to be screened at one time. Since each infectious phage encodes the random sequence expressed on its surface, a particular phage, when recovered from an affinity matrix, can be amplified by another round of infection. Thus, selector molecules immobilized on a solid support can be used to select peptides that bind to them. This procedure reveals a number of peptides that bind to the selector and that often display a common consensus amino acid sequence. Biological amplification of selected library members and sequencing allows the determination of the primary structure of the peptide(s).

Peptides are expressed on the tip of the filamentous phage M13, as a fusion protein with the phage surface protein pilus (at the N-terminus). Typically, a filamentous phage carries on its surface 3 to 5 copies of pili and therefore of the peptide. In such a system, no structural constraints are imposed on the N-terminus; the peptide is therefore free to adopt many different conformations, allowing for enhanced diversity.

In another aspect, synthetic libraries [Needels et al., Proc. Natl. Acad. Sci. USA 90:10700-4 (1993); Ohlmeyer et al., Proc. Natl. Acad. Sci. USA 90:10922-10926 (1993); Lam et al., International Patent Publication No. WO 92/00252; Kocis et al., International Patent Publication No. WO 9428028, each of which is incorporated herein by reference in its entirety], and the like can be used in screening assays. The LOPAC Library of Pharmacologically Active Compounds for Assay Validation and High Throughput Screening (Sigma, Catalog # SC001) may also be used in screening assays. In addition, a number of chemical libraries are available for screening from the Development Therapeutics program at The National Cancer Institute site on the worldwide web via Developmental Therapeutics Program (DTP) National Cancer Institute National Institutes of Health website gov/branches/dscb/div2 explanation.html. These include “NCI Diversity Set #1” and the “Approved Oncology Drugs Set.”

Alternatively, or in addition, the effect of a candidate compound may be tested in screens using immune cells, such as T_{H}17 cells obtained from tissues such as a mammalian gastrointestinal tract or differentiated from naive T cells isolated from, e.g., blood. One may, for example, assess the effects of a candidate compound on T_{H}17 activation and resultant release of inflammatory cytokines, such as IL17A, IL17F, and IL22. A positive candidate, if it were an inhibitor, e.g., would reduce the amount of IL17A, IL17F, and IL22 expressed.

Methods used to measure the effect of the candidate compound on T_{H}17 cells in general and on T_{H}17 cytokine expression in particular may include standard procedures known to those skilled in the art. The release of T_{H}17 cytokines (e.g., IL17A, IL17F, and IL22) cells can, for example, be measured by harvesting cellular supernatants and assaying same by immunoblotting or on the basis of biological activity present therein. The level of expression of a gene or gene product (protein) may furthermore be determined by a method selected from, but not limited to, cDNA microarray analyses, reverse transcription-polymerase chain reaction (RT-PCR), real time PCR and proteomics analysis. Other means such as electrophoretic gel analysis, enzyme immunoassays (ELISA assays), Western blots, dot blot analysis, Northern blot analysis and in situ hybridization may also be contemplated for use, although it is to be understood that the former assays that are noted (e.g. micrarrays, RT-PCR, real time PCR and proteomics analysis) provide a more sensitive, quantitative and reliable measurement of genes or gene products that are modulated by a candidate compound. Sequences of the genes or cDNA from which probes are made (if needed) for analysis may be obtained, e.g., from GenBank.

### In Vitro/In Vivo Methods

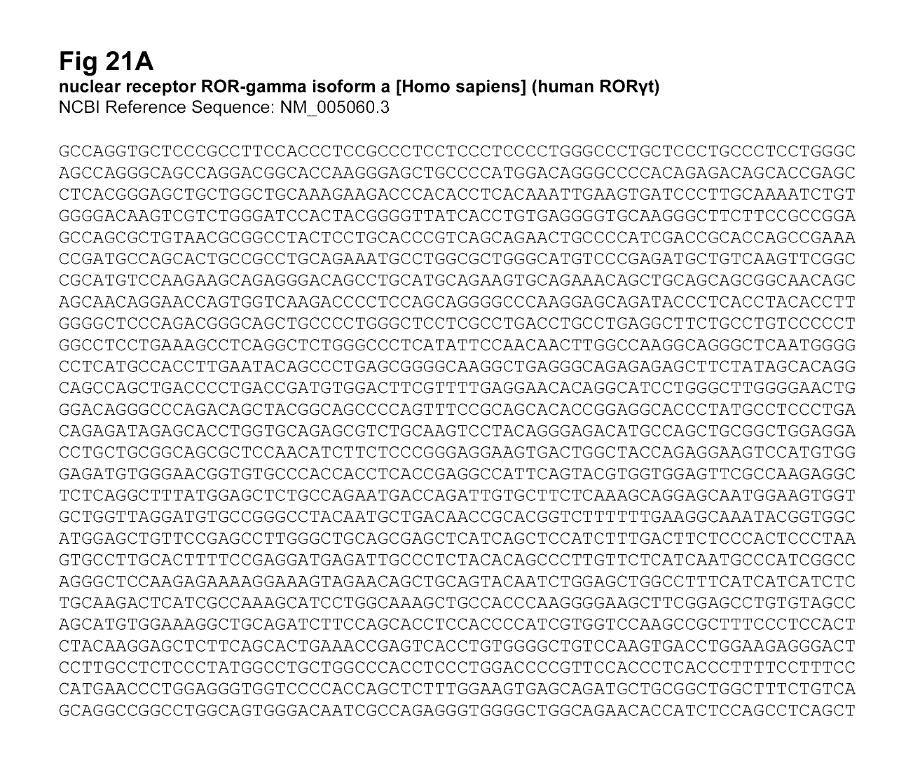

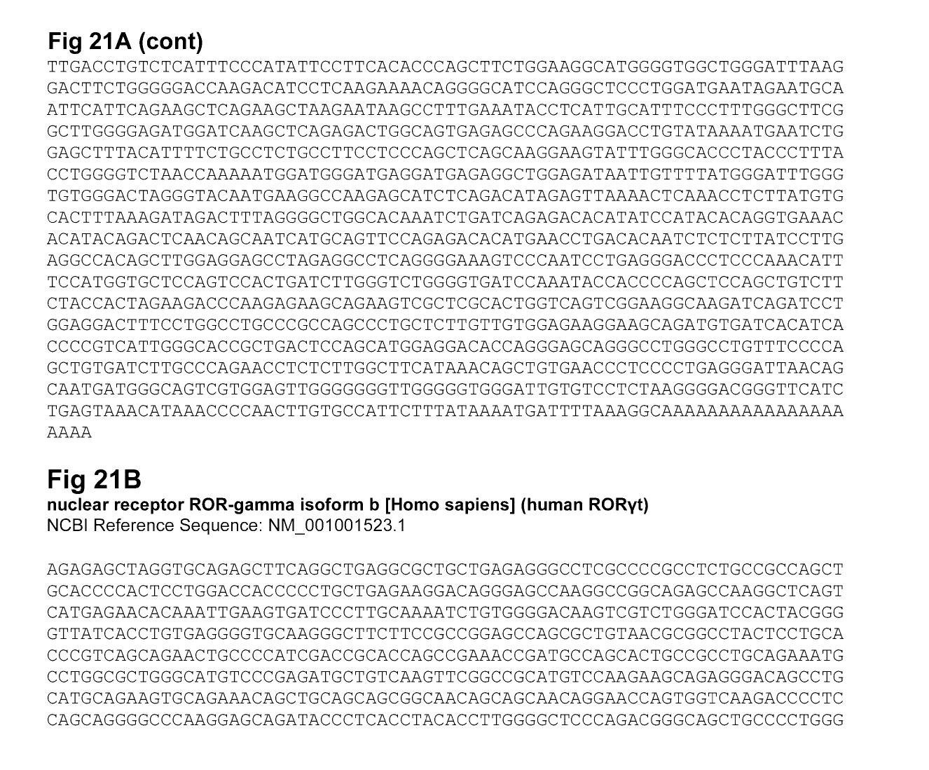

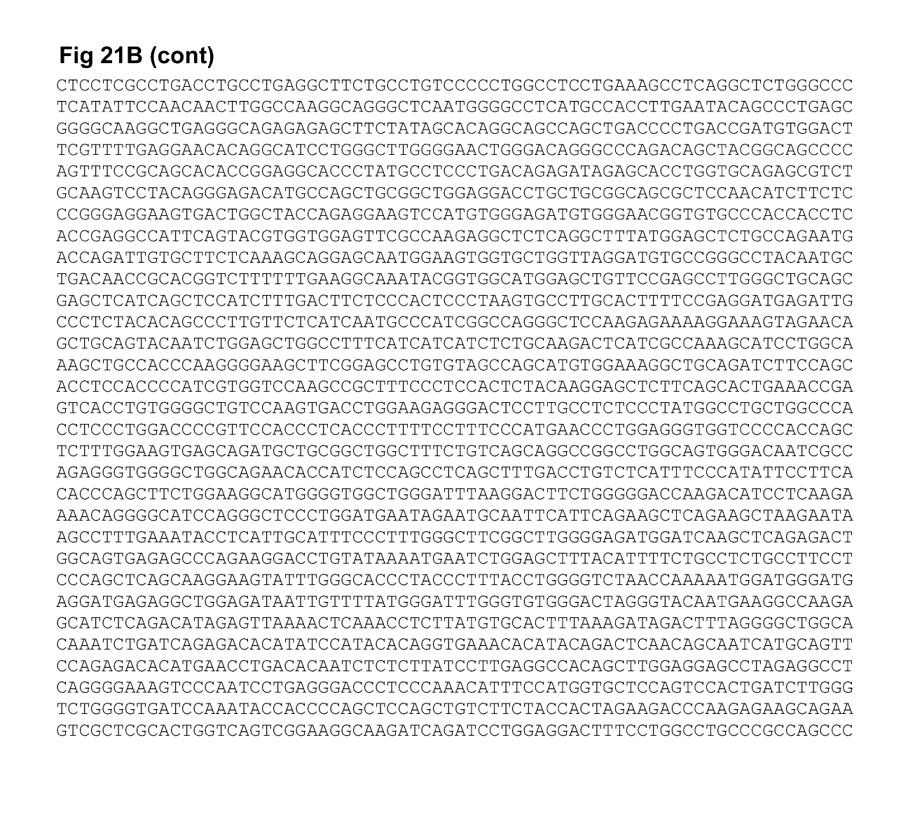

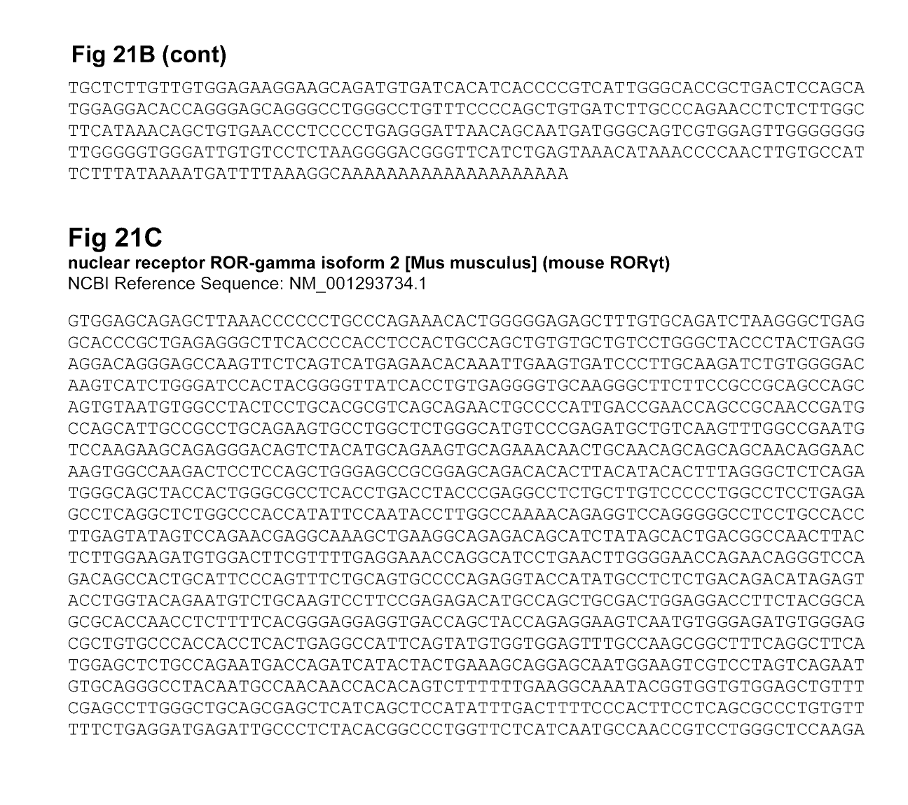

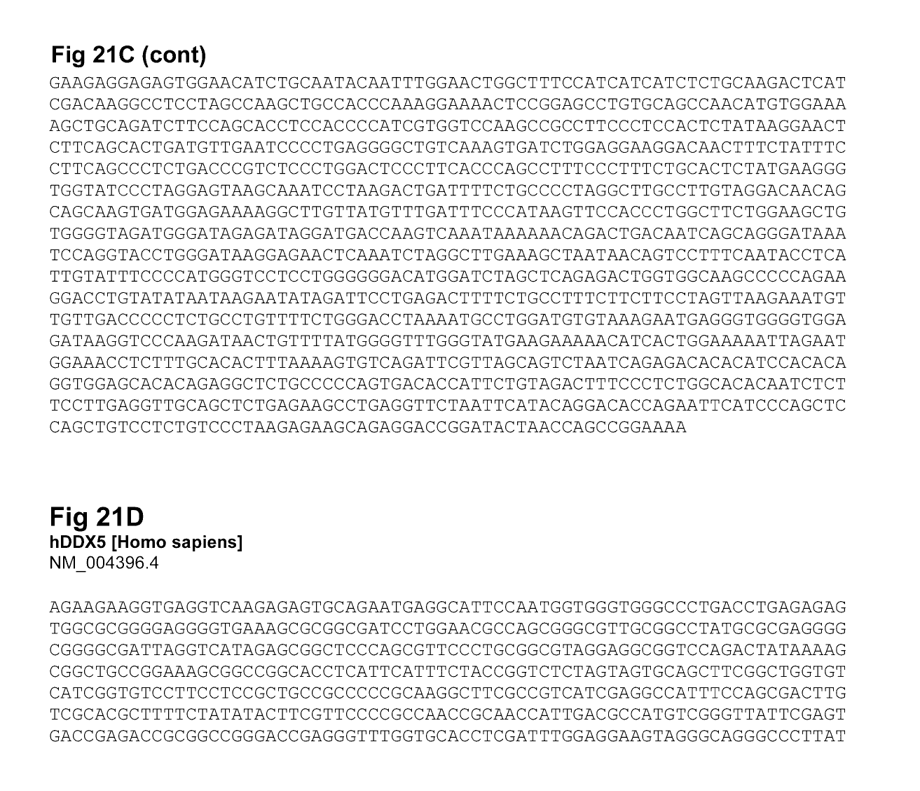

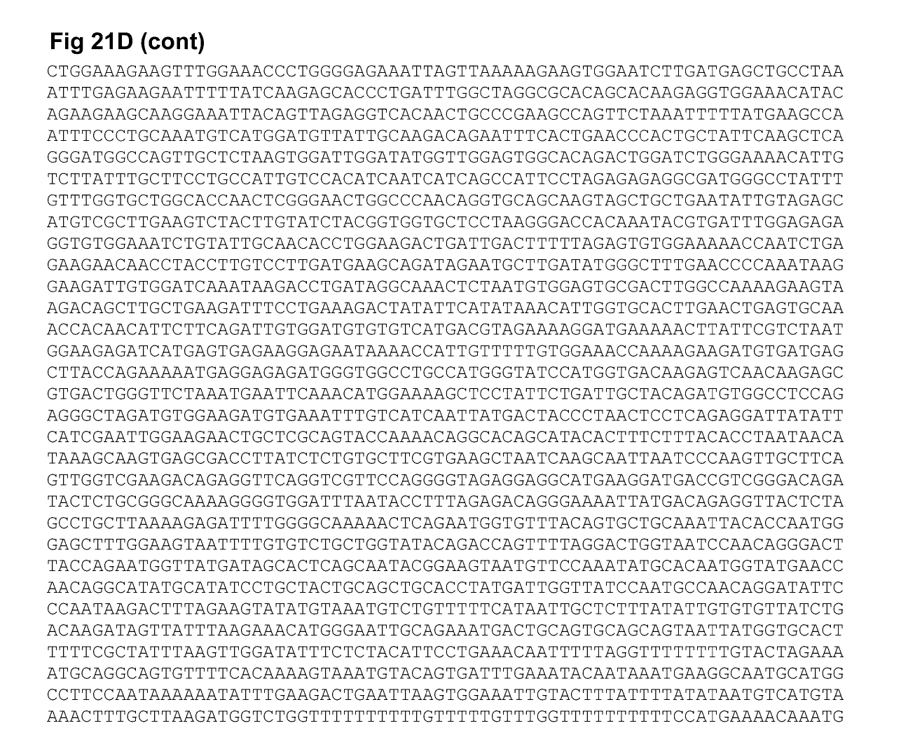

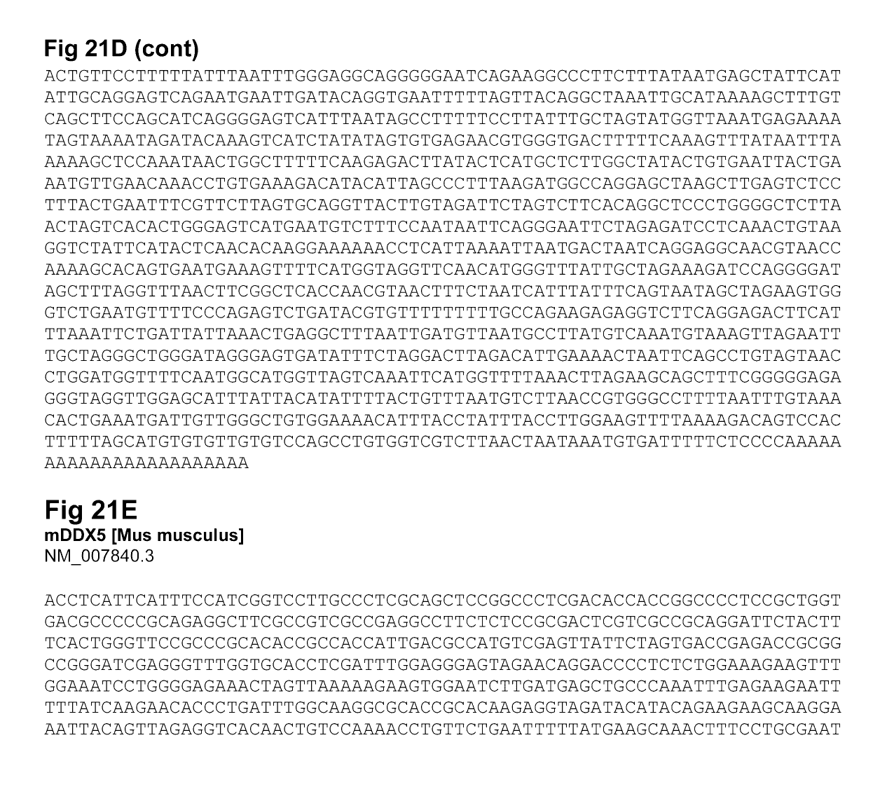

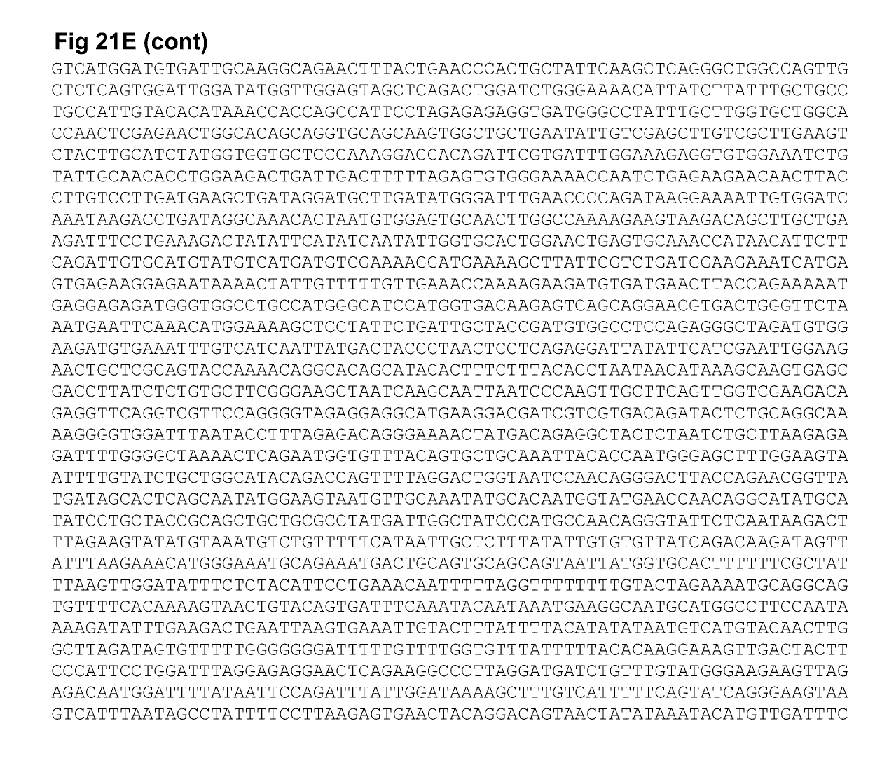

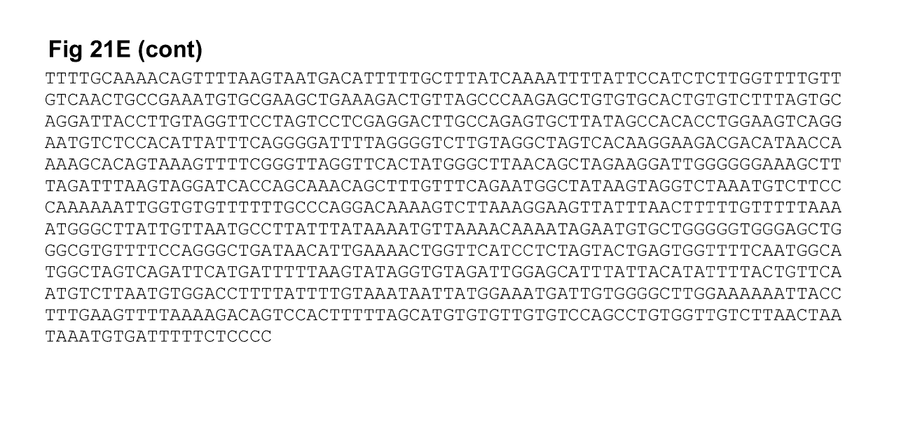

As described herein, the present invention is directed to a method for screening to identify agents that modulate (inhibit or enhance) RORγt, DDX5, and/or Rmrp activity, the method comprising inter alia the steps of: providing a polypeptide mixture comprising RORγt polypeptide, DDX5 polypeptide, and Rmrp ribonucleic acid (RNA) [in, e.g., a buffer with physiological pH 7.5 and comprising magnesium chloride (MgCl₂) and Adenosine Triphosphate (ATP)] and at least one candidate modulator agent and detecting RORγt/DDX5/Rmrp complex formation in the presence of the candidate modulator agent and comparing that to RORγt/DDX5/Rmrp complex formation in the absence of the candidate modulator agent, wherein a change in RORγt/DDX5/Rmrp complex formation in the presence of the candidate modulator agent relative to that detected in the absence of the candidate modulator agent indicates that the at least one candidate modulator agent is a modulator of RORγt polypeptide activity. Exemplary amino acid sequences of RORγt or a functional fragment thereof are presented in SEQ ID NOs: 1-3; exemplary amino acid sequences of DDX5 or a functional fragment thereof are presented in SEQ ID NOs: 4 and 5; and exemplary nucleic acid sequences of Rmrp or a functional fragment thereof are presented in SEQ ID NOs: 6 and 7. See also FIGS. 21A-E and GenBank Accession Number entries listed therein, which provide nucleic acid sequences corresponding to the indicated amino acid sequences, the entirety of each of which entries is incorporated herein by reference.

Co-crystallization of RORγt, DDX5, and/or Rmrp with compounds identified in screening methods described herein can be performed using methods known in the art.

Computer assisted three dimensional reconstruction of RORγt, DDX5, and/or Rmrp may also be used to identify and design inhibitors or activators of these molecules that modulate T_{H}17 cell activity or to provide guidance on which basis a more targeted screen can be designed.

Further to the above, once identified, active candidate agents (e.g., RORγt, DDX5, and/or Rmrp inhibitors) are assessed in secondary screens that may, for example, be cell-based assays. Cells useful for such assays include, without limitation, those cells described herein, including T_{H}17 cells which are isolated or derived from a mammal. T_{H}17 cells for use in the secondary assays can be isolated or derived from, for example, humans, other primates, mice, and rats. Secondary screens may also be performed in vivo, using animal model systems such as those described herein and known in the art. Such animal model systems include those which recapitulate aspects of human T_{H}17-mediated inflammatory conditions and autoimmune diseases including, without limitation, Crohn's disease, ulcerative colitis, multiple sclerosis, rheumatoid arthritis, and psoriasis. With respect to treatment of T_{H}17-mediated inflammatory conditions and autoimmune diseases, it is envisioned that inhibitors of RORγt, DDX5, and/or Rmrp activity will confer benefit to subjects suffering from one of these conditions.

In another embodiment, Rmrp mimics are envisioned that function to either inhibit/suppress T_{H}17 cell activity or that function to enhance/promote T_{H}17 cell activity.

Also envisioned herein are applications wherein modulators identified that enhance RORγt, DDX5, and/or Rmrp activity are used to treat various cancers such as, without limitation, gastric adenocarcinoma, lung cancer, ovarian cancer, melanoma, glioblastoma, and pancreatic cancer. In gastric adenocarcinoma, for example, low levels of intratumoral IL-17 expression may be indicative of poor prognosis in gastric adenocarcinoma patients (Chen et al. 2011, Int J Biol Sci 7:53-60). In patients with lung cancer, the accumulation of T_{H}17 cells in malignant plural effusion (MPE) is predictive of improved patient survival (Yang et al. 2015, Oncol Rep 33:478-484). In ovarian cancer patients, inhibition of T_{H}17 cells represents a novel immune evasion mechanism (Kryczek et al. 2009, Blood 114:1141-1149). Reduced T_{H}17 activity has also been implicated in melanoma and pancreatic cancer (Ye et al. 2013, Am J Pathol 182:10-20). Accordingly, enhancers or promoters of RORγt, DDX5, and/or Rmrp activity are envisioned as having utility in the treatment of these cancers and others wherein reduced T_{H}17 activity is correlated with poor prognosis.

As described herein, a cell-based assay may be performed essentially as a method for inhibiting T_{H}17 cell activation, the method comprising the steps of: contacting a population of T_{H}17 cells with either an active candidate agent (e.g., a RORγt, DDX5, and/or Rmrp activity inhibitor identified in a primary screen) or a control substance and evaluating the ability of the active candidate agent relative to that of the control substance to reduce or inhibit T_{H}17 cell activity, wherein if the active candidate agent reduces or inhibits T_{H}17 cell activity relative to the contol substance, the active candidate agent is identified as an inhibitor of T_{H}17 cell activity and is thus confirmed as a bona fide RORγt, DDX5, and/or Rmrp activity inhibitor in a cellular context. As described herein, secondary cell-based assays can be performed in cell culture (in vitro) or in the context of an animal (in vivo). As described herein, an active candidate agent may be identified by analyses based on computer modeling of three dimensional structure and/or by primary screens of libraries.

As taught herein, in vitro T_{H}17 cell activation can be evaluated or measured by detecting an increase, for example, in release of T_{H}17 cytokines, such as IL17A, IL17F, and IL22. Secondary cell-based assays directed to identifying or confirming that a candidate agent is a RORγt, DDX5, and/or Rmrp inhibitor call for contacting a population of naive T cells or T_{H}17 cells with a potential RORγt, DDX5, and/or Rmrp inhibitor or inhibitors that is/are added before, concurrently, or after contacting the T_{H}17 cells with target cells and/or T_{H}17 promoting conditions or before, concurrently, or after exposing the naive T cells to T_{H}17 promoting conditions. Experimental protocols that can be used to measure T_{H}17 activity and reduction or inhibition thereof are described in detail herein and are understood in the art.

As described herein, assays may be performed in a vessel or in cell culture. Suitable vessels are known in the art and include, without limitation, a test tube or plurality of same or a multi-well plate.

### Candidate Compounds and Agents

As used herein, an “agent”, “candidate compound”, or “test compound” may be used to refer to, for example, nucleic acids (e.g., DNA and RNA), carbohydrates, lipids, proteins, peptides, peptidomimetics, small molecules, small organic molecules, and other drugs. Candidate agents or compounds may be assayed individually or as a plurality. A plurality of candidate compounds may comprise a library of candidate compounds. Such libraries my comprise a plurality of small molecules, small organic molecules, or a chemical library. Nucleic acids envisioned include single-stranded and double-stranded DNA or RNA. An agent may also refer to short hairpin RNA (shRNA), small interfering RNA (siRNA), and neutralizing and/or blocking antibodies. Anti-sense oligonucleotides comprising single-stranded DNA specific for, Rmrp, for example, are envisioned herein.

A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. In a particular embodiment, shRNA or siRNA is designed and used to inhibit RORγt, DDX5, or Rmrp.

Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene.

siRNAs and modified RNAs are envisioned as having utility for either inhibiting or enhancing DDX5/RORγt complex formation. Either approach is envisioned as having potential for therapeutic intervention. In one embodiment, such siRNAs and modified RNAs could be used to screen for better modulators of interaction, and such could be delivered to enhance T_{H}17 cell differentiation so as to change the tumor microenvironment.

Exemplary modulators of DDX5 helicase activity include ATP analogs, which would be anticipated to bind to the ATP binding pocket of DDX5.

As described herein, an agent identified using the method of the present invention that is a “modulator of T_{H}17 cell activity” is defined as an agent that is capable of modulating (e.g., increasing or decreasing) activity of T_{H}17 cells. Such an agent may be identified by its ability to effect a change in expression of T_{H}17 cytokines, such as IL17A, IL17F, and IL22. As detailed below, experimental protocols of utility in evaluating the above indicators of T_{H}17 cell activity are described in detail herein and are understood in the art. Such experimental protocols, include, but are not limited to, measuring IL17A, IL17F, and IL22 mRNA and/or protein expression.

As taught herein, the change effected by an agent that is a modulator of T_{H}17 cell activity is determined relative to that of a population of, for example, T_{H}17 cells incubated in parallel in the absence of the agent or in the presence of a control agent (as described below), either of which is analogous to a negative control condition.

The term “control substance”, “control agent”, or “control compound” as used herein refers a molecule that is inert or has no activity relating to an ability to modulate a biological activity. With respect to the present invention, such control substances are inert with respect to an ability to modulate T_{H}17 cell activity or cytokine release therefrom. Exemplary controls include, but are not limited to, solutions comprising physiological salt concentrations.

It is to be understood that agents capable of modulating T_{H}17 cell activity or cytokine release therefrom, as determined using in vitro methods described herein, are likely to exhibit similar modulatory capacity in applications in vivo.

Modulatory agents identified using the screening methods of the present invention and compositions thereof can thus be administered for therapeutic treatments. In therapeutic applications, modulatory agents that inhibit T_{H}17 cell activity and compositions thereof are administered to a patient susceptible to or suffering from a T_{H}17-mediated condition or disorder in an amount sufficient to at least partially arrest a symptom or symptoms of the condition or disorder and its complications. An amount adequate to accomplish this is defined as a “therapeutically effective amount or dose.” Amounts effective for this use will depend on the severity of the disease and the weight and general state of the patient.

Examples of T_{H}17-mediated inflammatory conditions and autoimmune diseases include, without limitation, Crohn's disease, ulcerative colitis, multiple sclerosis, rheumatoid arthritis, and psoriasis.

In an alternative embodiment, modulatory agents that enhance or promote T_{H}17 cell activity and compositions thereof are administered to a patient susceptible to or suffering from a condition wherein T_{H}17 cell activity is reduced or impaired, which reduction or impairment is viewed as associated with the condition. An exemplary such condition is cancer, including gastric adenocarcinoma, lung cancer, ovarian cancer, melanoma, and pancreatic cancer. Under such circumstances, the enhancer or promoter of T_{H}17 cell activity is administered in an amount sufficient to at least partially arrest a symptom or symptoms of the condition or disorder and its complications.

The basic molecular biology techniques used to practice the methods of the invention are well known in the art, and are described for example in Sambrook et al., 1989, Molecular Cloning: A Laboratory Manual, Cold Spring Harbor Laboratory, New York; Ausubel et al., 1988, Current Protocols in Molecular Biology, John Wiley & Sons, New York; and Ausubel et al., 2002, Short Protocols in Molecular Biology, John Wiley & Sons, New York).

### Agents Identified by the Screening Methods of the Invention

The invention provides methods for identifying agents (e.g., candidate compounds or test compounds) that modulate (inhibit or promote) T_{H}17 cell activity. Agents that are capable of inhibiting T_{H}17 activity, for example, as identified by screening methods described herein, are useful as candidate therapeutics for T_{H}17-mediated inflammatory conditions and autoimmune diseases. This assertion is based on results presented herein that demonstrate for the first time that RORγt, DDX5, and Rmrp form a complex that contributes to transcriptional regulation of IL17A, IL17F, and IL22 in T_{H}17 cells.

A list of T_{H}17-mediated inflammatory conditions and autoimmune diseases that may be treated using an agent identified using a method described herein includes, without limitation: Crohn's disease, ulcerative colitis, multiple sclerosis [and the animal model thereof, experimental autoimmune encephalomyelitis (EAE)], rheumatoid arthritis [and the animal model thereof, collagen-induced arthritis (CIA)], inflammatory bowel disease (IBD) [and the animal model thereof, transfer colitis], cancer as it relates to inflammation [see, for example, Grivennikov et al. (2010, Cell 140:883-899), the entire content of which is incorporated herein by reference], sprue, food allergies, graft-versus-host disease, asthma, psoriasis, diabetes, uveitis, bronchitis, allergic rhinitis, chronic obstructive pulmonary disease, atherosclerosis, and H pylon infections and ulcers resulting from such infections.

Diseases and/or conditions wherein T_{H}17 cells contribute also include, without limitation: cancer in general [see, for example, Grivennikov et al. (2010, Cell 140:883-899)], including certain types of prostate cancer [see, for example, Wang et al. (2016, Nat Medicine 22:488-496), the entire content of which is incorporated herein by reference], as alluded to above, and microbial infections, such as those caused by Candida and Mycobacterium. See, for example, Okada et al. (2015, Science 349:606-613), the entire content of which is incorporated herein by reference. It is, therefore, envisioned that an agent identified using a method described herein could be used to enhance T_{H}17-mediated activity to promote anti-tumor immunity and anti-microbial responses and thus, such an agent would be useful for treating such conditions. It is, moreover, also envisioned that an agent identified using a method described herein could be used to inhibit T_{H}17-mediated activity in the context of certain cancers, including certain types of prostate cancer [see, for example, Wang et al. (2016, Nat Medicine 22:488-496)]. Inhibition of Th17 may also be therapeutic for colorectal cancer. The choice of whether to use an agent that acts as an enhancer or inhibitor of T_{H}17-mediated activity may be determined by a skilled practitioner based on the type of cancer being treated and the stage of the cancer, consideration of standards for clinical care in the field, and advances in the scientific literature. Appropriate therapy will also be determined based on the nature of the tumor microenvironment, which will vary among individuals. Given that, a personalized or precision approach will likely be implemented by a skilled practitioner when considering whether to administer an enhancer or inhibitor of Th17 cells.

Examples of agents, candidate compounds or test compounds include, but are not limited to, nucleic acids (e.g., DNA and RNA), carbohydrates, lipids, proteins, peptides, peptidomimetics, small organic molecules, small molecules, and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82- 84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

### Therapeutic Uses of Agents Identified

The invention provides for treatment of T_{H}17-mediated inflammatory conditions and autoimmune diseases by administration of a therapeutic agent identified using the above-described methods. Such agents include, but are not limited to proteins, peptides, protein or peptide derivatives or analogs, antibodies, nucleic acids, and small molecules.

The invention provides methods for treating patients afflicted with a T_{H}17-mediated inflammatory condition or autoimmune disease comprising administering to a subject an effective amount of a compound identified by the method of the invention. In a particular aspect, the compound is substantially purified (e.g., substantially free from substances that limit its effect or produce undesired side-effects). The subject is particularly an animal, including but not limited to animals such as cows, pigs, horses, chickens, cats, dogs, etc., and is more particularly a mammal, and most particularly a human. In a particular embodiment, a non-human mammal or a human is the subject.

Formulations and methods of administration that can be employed when the compound comprises a nucleic acid are described herein and known in the art; additional appropriate formulations and routes of administration are described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), and construction of a nucleic acid as part of a retroviral or other vector. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a particular embodiment, modulators of DDX5 identified using methods described herein and Rmrp mimicks identified that inhibit T_{H}17 cell activity may be delivered specifically or preferentially to the gastrointestinal tract, and particularly to the large intestine. Methods for specific and/or preferential delivery to the gastrointestinal tract, and particularly to the large intestine, are known in the art. In another particular embodiment, modulators of DDX5 identified using methods described herein and Rmrp mimicks identified that enhance or promote T_{H}17 cell activity may be delivered specifically or preferentially to a tumor or tumor microenvironment or immune cells to target the tumor via, for example, hypodermic needle or port.

In a particular embodiment thereof, a physiologically compatible carrier or excipient compatible with oral and/or anal administration is used. Exemplary buffers compatible with oral administration include solutions that are physiologically compatible such as, for example, sterile normal saline or a sterile saline-based gelatin or matrix. Normal saline is typically defined as a solution of 0.90% weight/volume of NaCl, about 300 mOsm/L or about 9.0 grams NaCl per liter of water. In a particular embodiment, oral administration is achieved using an encapsulated means, wherein the capsule is designed to dissolve or disintegrate in the small and/or large intestine. Exemplary buffers compatible with anal administration comprise solutions that are physiologically compatible such as, for example, normal saline, saline-based gelatin, oleaginous (fatty) bases [e.g., theobroma oil (cocoa butter) and synthetic triglycerides], and water soluble or miscible bases (e.g., glycerinated gelatin and polyethylene glycol polymers).

In another particular embodiment, modulators of DDX5 identified using methods described herein are targeted specifically to T cells or are expressed specifically in T cells.

In yet another particular embodiment, modulators of Rmrp identified using methods described herein are targeted specifically to T cells or are expressed specifically in T cells. In that Rmrp is a developmentally important gene and deletion of this gene or ablation of its function results in embryonic lethality in mice, targeted delivery of Rmrp modulators to T cells is desirable and thereby, spares alteration of Rmrp essential physiological functions in other cellular contexts.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, e.g., an inflammatory site, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

Methods suitable for delivery of anti-sense oligonucleotides have been described in, for example, Monteleone et al. (2015, N Engl J Med 372:1104-1113), the entire content of which is incorporated herein by reference. As described therein, an oral SMAD7 anti-sense oligonucleotide, mongersen, has been used successfully to target ileal and colonic SMAD7 and thereby treat subjects afflicted with Crohn's Disease.

### Pharmaceutical Compositions

The present invention also provides pharmaceutical compositions. Such compositions comprise a therapeutically effective amount of an agent and a pharmaceutically acceptable carrier. In a particular embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, incorporated in its entirety by reference herein. Such compositions will contain a therapeutically effective amount of the compound, preferably in purified form, together with a suitable amount of carrier so as to provide the form for proper administration to the subject. The formulation should suit the mode of administration.

In a particular embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of a T_{H}17-mediated inflammatory condition or autoimmune disease (e.g., Crohn's disease, ulcerative colitis, multiple sclerosis, rheumatoid arthritis, and psoriasis) can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Suppositories generally contain active ingredient in the range of 0.5% to 10% by weight; oral formulations preferably contain 10% to 95% active ingredient. Effective doses may be extrapolated from dose-response curves derived from in vitro or animal model test systems.

### Nucleic Acids

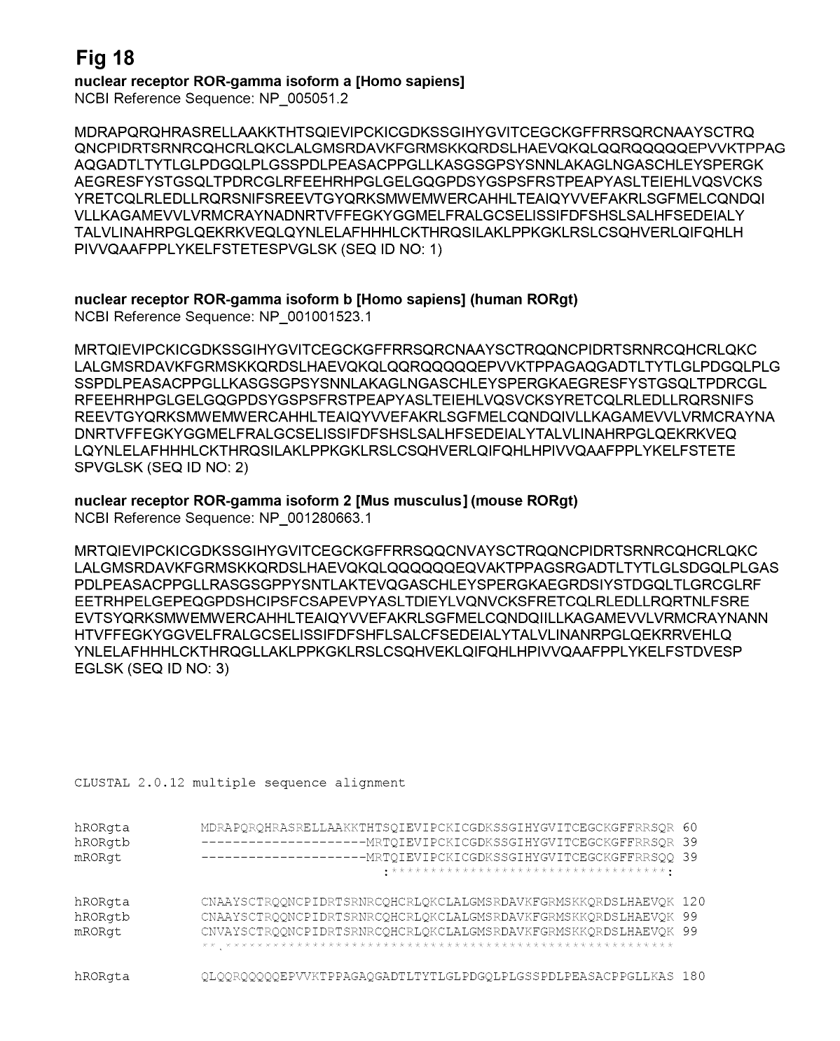

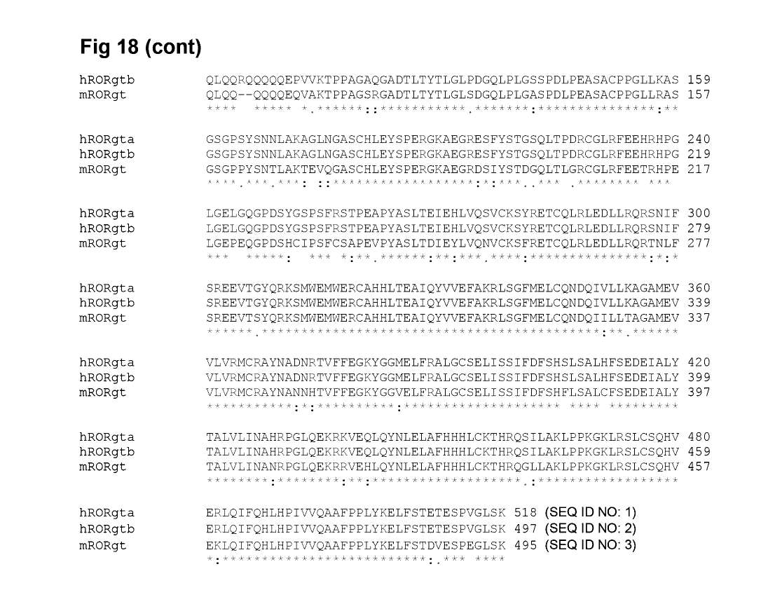

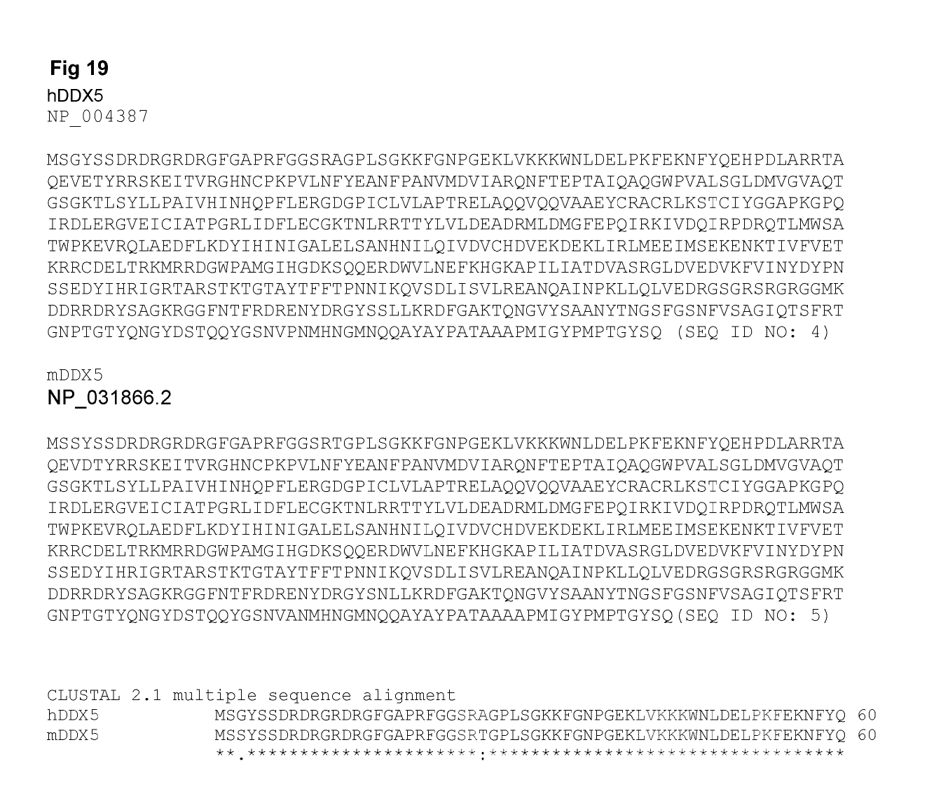

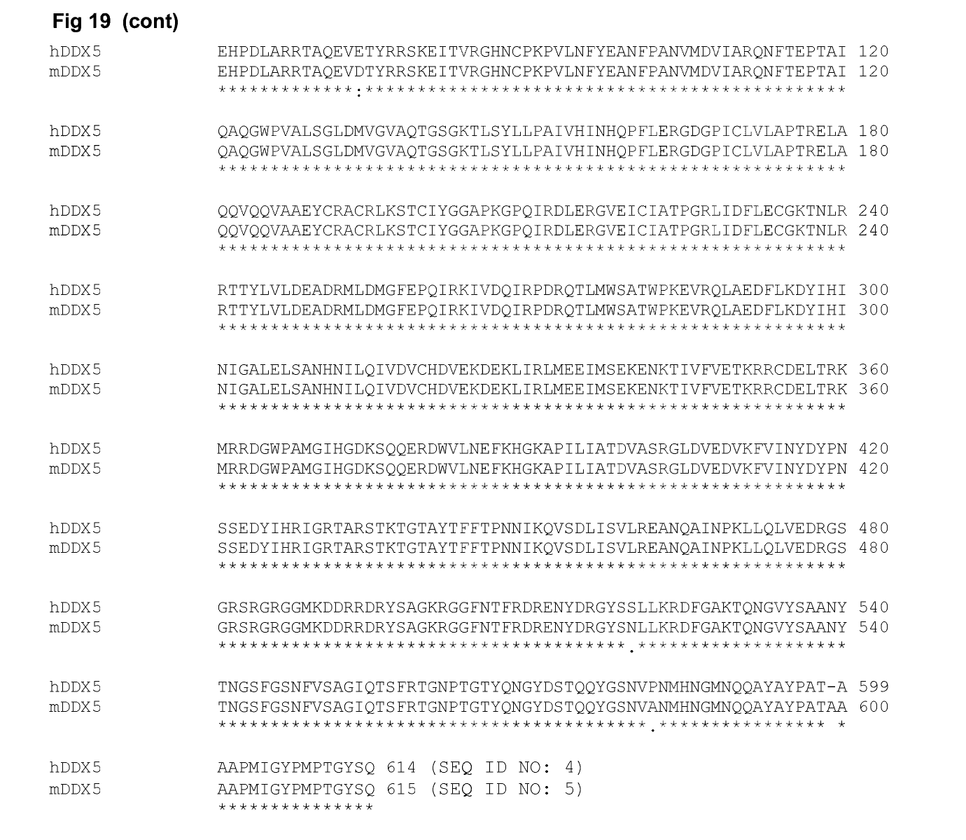

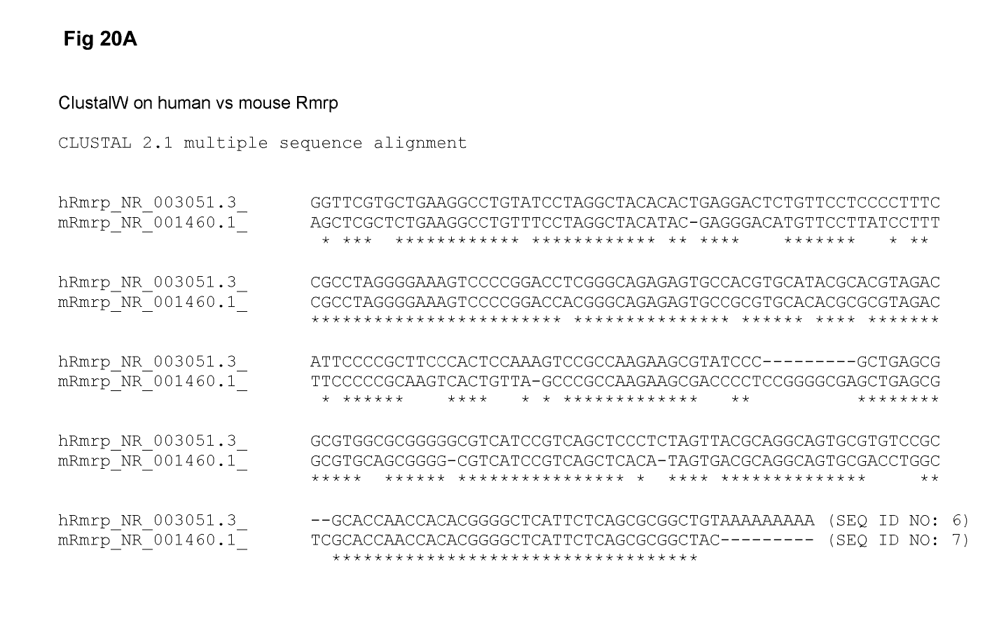

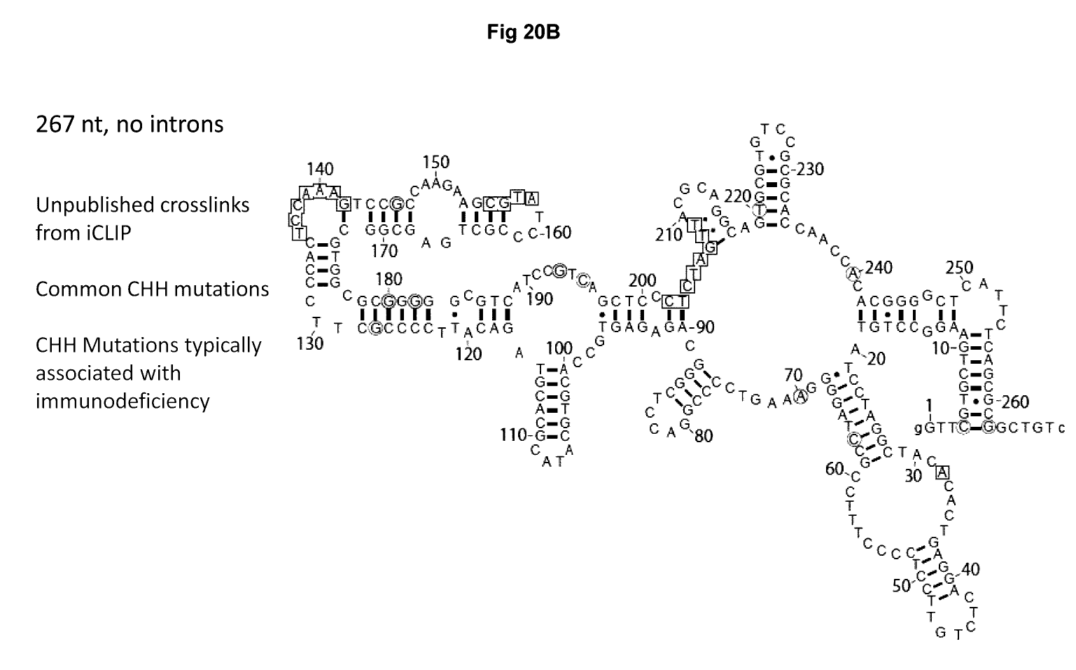

The invention provides methods of identifying agents capable of modulating T_{H}17 cell activity. Accordingly, the invention encompasses administration of a nucleic acid encoding a peptide or protein capable of modulating interactions among RORγt, DDX5, and Rmrp and/or activity of at least one RORγt, DDX5, and Rmrp, as well as antisense sequences or catalytic RNAs capable of interfering with RORγt, DDX5, and/or Rmrp expression and/or activity. Exemplary amino acid sequences of RORγt or a functional fragment thereof are presented in SEQ ID NOs: 1-3; exemplary amino acid sequences of DDX5 or a functional fragment thereof are presented in SEQ ID NOs: 4 and 5; and exemplary amino acid sequences of Rmrp or a functional fragment thereof are presented in SEQ ID NOs: 6 and 7. See also FIGS. 18, 19, and 20 and citations listed for nucleic acid sequences, the entire content of each of which is incorporated herein by reference.

Any suitable methods for administering a nucleic acid sequence available in the art can be used according to the present invention.

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, the compound comprises a nucleic acid encoding a peptide or protein or nucleic acid sequence capable of modulating T_{H}17 cell activity, such nucleic acid being part of an expression vector that expresses the peptide or protein or nucleic acid sequence in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). These retroviral vectors have been modified to delete retroviral sequences that are not necessary for packaging of the viral genome and integration into host cell DNA. The nucleic acid encoding a desired polypeptide to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdrl gene to hematopoietic stem cells in order to make the stem cells more resistant to chemotherapy. Other references illustrating the use of retroviral vectors in gene therapy are: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses may also be used effectively in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a particular embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a particular embodiment, the cell used for gene therapy is autologous to the subject that is treated.

In another embodiment, the nucleic acid to be introduced for purposes of gene therapy may comprise an inducible promoter operably linked to the coding region, such that expression of the nucleic acid is controllable by adjusting the concentration of an appropriate inducer of transcription.

Direct injection of a DNA coding for a peptide or protein capable of modulating T_{H}17 cell activity may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection.

Unless defined otherwise, all technical and scientific terms used herein have the same meaning as commonly understood by one of ordinary skill in the art to which this invention belongs.

It is to be understood that this invention is not limited to particular assay methods, or test agents and experimental conditions described, as such methods and agents may vary. It is also to be understood that the terminology used herein is for the purpose of describing particular embodiments only, and is not intended to be limiting, since the scope of the present invention will be limited only the appended claims.

### EXAMPLE I

### Materials and Methods

Data reporting. No statistical methods were used to predetermine sample size. The experiments were not randomized. In vivo transfer colitis and EAE mouse experiments were blinded, but cell culture and in vitro studies were not.

Mice. EEF1A1-LSL.EGFPL10 (lox-stop-lox-EGFP-L10 knockin at the Eflal locus) transgenic mice, RORγ/γt-deficient animals and Dcbc5fl/fl mice have been previously described (31,47,48). Conditional mutant mice were bred to CD4-Cre transgenic animals (Taconic) and maintained on the C57BL/6 background. Heterozygous mice were bred to yield 6-8-week-old Dcbc5+/+CD4-Cre+ (subsequently referred to as wild type) and Ddx5fl/flCD4-Cre+ (referred to as DDX5-T) littermates for experiments examining DDX5 in peripheral T-cell function. DDX5 conditional mutant mice were also bred to IL7R-Cre transgenic animals (Jackson Laboratory) (with Ddx5 deleted in common early lymphoid progenitors; referred to as DDX5-clpKO) for experiments examining DDX5 functions during T-cell development in the thymus. RmrpG270T knock-in mice were generated using CRISPRCas9 technology by the Rodent Genetic Engineering Core (RGEC) at New York University Langone Medical Center. Guide RNA and homology directed repair donor template sequences are provided in Supplementary Table 1. Heterozygous crosses provided Rmrp+/+ (wild-type) and RmrpG270T/G270Tlittermates for in vivo studies. All animal procedures were in accordance with protocols approved by the Institutional Animal Care and Use Committee of the New York University School of Medicine (Animal Welfare Assurance number: A3435-01).

In vivo studies. Steady-state small intestines were collected for isolation of lamina propria mononuclear cells as previously described45. For detecting SFB colonization, SFB-specific 16S primers were used (49). Universal 16S and/or host genomic DNA was quantified simultaneously to normalize SFB colonization of each sample. All primer sequences are listed in Supplementary Table 1.

For the adoptive transfer model of colitis, 5×105 CD4+CD25−CD44low CD45RBhiCD62Lhi T cells were isolated from mouse splenocytes by FACS sorting and administered i.p. into Rag2−/− mice as previously described (50). Animal weights were measured approximately weekly. Between weeks seven and eight, large intestines were collected for H&E staining and isolation of lamina propria mononuclear cells as previously described (45). The H&E slides from each sample were examined in a double-blind fashion. The histology scoring (scale 0-24) was based on the evaluation of criteria described previously (51).

For induction of active EAE, each mouse was immunized subcutaneously on day 0 with 100 μg of MOG35-55 peptide, emulsified in CFA (Complete Freund's Adjuvant supplemented with additional 2 mg ml−1 Mycobacterium tuberculosis), and injected i.p. on days 0 and 2 with 100 ng per mouse of pertussis toxin (Calbiochem). The EAE scoring system was as follows: 0, no disease; 1, limp tail; 2, weak/partially paralyzed hind legs; 3, completely paralyzed hind legs; 4, complete hind and partial front leg paralysis; 5, complete paralysis/death.

In transfer colitis and EAE experiments, animals of different genotypes were co-housed and weighed and scored blindly. For statistical power level of 0.8, probability level of 0.05, anticipated effect size of 2, minimum sample size per group for two-tailed hypothesis is 6. Two-tailed unpaired Student's t-test was performed using Prism (GraphPad Software). A P value of less than 0.05 was treasted as a significant difference. All experiments were performed at least twice.

In vitro T-cell culture and phenotypic analysis. Mouse T cells were purified from lymph nodes and spleens of 6-8-week-old mice, by sorting live (DAPI−), CD8−CD19−CD4+CD25−CD44low/intCD62L+ naive T cells using a FACSAria (BD). Detailed antibody information is provided in Supplementary Table 1. Cells were cultured in Iscove's Modified Dulbecco's Medium (IMDM, Sigma) supplemented with 10% heat-inactivated FBS (Hyclone), 50 U penicillin-streptomycin (Invitrogen), 4 mM glutamine and 50 μM β-mercaptoethanol. For T-cell polarization, 200 μl of cells was seeded at 0.3×105 cells per ml in 96-well plates precoated with goat anti-hamster IgG at a 1:20 dilution of stock (1 mg ml—1, MP Biomedicals). Naive T cells were activated with anti-CD3ε (2.5 μg ml−1) and anti-CD28 (10 μg ml−1). Cells were cultured for 4-5 days under T_{H}17-polarizing conditions (0.1-0.3 ng ml−1 TGF-β, 20 ng ml−1 IL-6), TH1−(10 ng ml−1 IL-12, 10 U ml−1 IL-2), TH2−(10 ng ml−1 IL-4, 10 U ml−1 IL-2), or Treg−(5 ng ml−1 TGF-β, 10 U ml−1 IL-2) conditions.

Human T cells were isolated from peripheral blood of healthy donors using anti-human CD4 MACS beads (Miltenyi). Human CD4+ T cells were cultured in 96-well U bottom plates in 10 U ml−1 of IL-2, 10 ng ml−1 of IL-1(3, 10 ng ml−1 of IL-23, 1 μg ml−1 of anti-IL-4, 1 μg ml−1 of anti-IFNγ and anti-CD3/CD28 activation beads (LifeTechnologies) at a ratio of 1 bead per cell, as previously described (52).

For cytokine analysis, cells were incubated for 5 h with phorbol 12-myristate 13-acetate (PMA) (50 ng ml−1; Sigma), ionomycin (500 ng ml−1; Sigma) and GolgiStop (BD). Intracellular cytokine staining was performed according to the manufacturer's protocol (Cytofix/Cytoperm buffer set from BD Biosciences and FoxP3 staining buffer set from eBioscience). A LSR II flow cytometer (BD Biosciences) and FlowJo (Tree Star) software were used for flow cytometry and analysis. Dead cells were excluded using the Live/Dead fixable aqua dead cell stain kit (Invitrogen).

Nucleic acid reagents and T-cell transduction. Custom Rmrp and predesigned Malatl Stellaris RNA fluorescence in situ hybridization (FISH) probes were purchased from BiosearchTech and used to label mouse Rmrp and Malatl RNA in cultured T_{H}17 cells according to the manufacturer's protocol. Control and human DDX5-specific short interfering RNAs (siRNAs) were obtained from Cell Signaling. Synthesis of ASOs was performed as previously described (53). All ASOs were 20 nucleotides in length and had a phosphorothioate backbone. The ASOs had five nucleotides at the 5′ and 3′ ends modified with 2′-O-methoxyethyl (MOE) for increased stability. ASOs and siRNA sequences are provided in Supplementary Table 1. siRNA and ASOs were introduced into mouse T_{H}17 cells by Amaxa nucleofection as previously described (8).

Wild-type and helicase-dead mutant DDX5 were described previously (54). DDX5 and Rmrp were subcloned into the mouse stem-cell virus (MSCV) Thyl.1 vectors for retroviral overexpression and rescue assays in T cells. Retrovirus production was carried out in Plat-E cells (Cell Biolabs, Inc., not tested for mycoplasma) as previously described (55). Spin transduction was performed 24 h after in vitro T-cell activation by centrifugation in a Sorvall Legend RT at 700 g for 90 min at 32° C. Aqua−Thyl.1+ live and transduced cells were analysed by flow cytometry after 5 days of culture in T_{H}17-polarizing conditions.

RORγt transcriptional activity in polarized T_{H}17 cells. A ROR luciferase reporter was constructed with four RORE sites replacing the Gal4 (UAS) sites from the pGL4.31 vector (luc2P/GAL4 UAS/Hygro) from Promega (C935A) as described in ref. 56. Naive CD4+ T cells were cultured in T_{H}17-polarizing conditions for 72 h. Nucleofection (Amaxa Nucleofector 4D, Lonza) was then used to introduce 1 μg RORE-firefly luciferase reporter construct and 1 μg control Renilla luciferase construct according to the manufacturer's instructions. Luciferase activity was measured using the dual luciferase reporter kit (Promega) at 24 h after transfection. Relative luciferase units (RLU) were calculated as a function of firefly luciferase reads over those of Renilla luciferase. DMSO or 2 μM RORγ inhibitor (ML209) were used in luciferase experiments as described in ref. 57.

Co-immunoprecipitation and mass spectrometry. Cultured T_{H}17 cells (100×10⁶) were lysed in 25 mM Tris (pH8.0), 100 mM NaCl, 0.5% NP-40, 10 mM MgCl₂, 10% glycerol, 1× protease inhibitor and PhosphoSTOP (Roche) on ice for 30 min, followed by homogenization with a 25-gauge needle. The RORγ/γt-specific antibody used for pull-down assays was previously described (8). Co-immunoprecipitated complexes were collected with protein G dynabeads (Dynal, Invitrogen). Detailed antibody information is provided in Supplementary Table 1. Mass spectrometry and the Mascot database search to identify protein complex composition were both performed by the Central Proteomics Facility at the Dunn School of Pathology, Oxford, UK.

Ribosome TRAP-seq, RIP-seq and RNA-seq. Twenty million cells cultured in T_{H}17-polarizing conditions for 48 h were lysed in 10 mM HEPES (pH7.4), 150 mM KCl, 5 mM MgCl₂, 0.5 mM dithiothreitol (DTT), 100 μg ml−1 cycloheximide, 1% NP-40, 30 mM DHPC, 1× protease inhibitor and PhosphoSTOP (Roche). Ribosome-TRAP immunoprecipitation was first performed using 2 μg of anti-GFP antibody (Invitrogen) and collected in 20 μl of protein G magnetic dynabeads. The supernatant was removed for subsequent RIP pull-down using anti-DDX5 (Abcam) or anti-RORγt antibodies and collected with protein G dynabeads. TRAPseq samples were washed with high-salt wash buffer (10 mM HEPES (pH7.4), 350 mM KCl, 5 mM MgCl₂, 1% NP-40, 0.5 mM DTT and 100 μg ml−1 cycloheximide). RIP-seq samples were washed three times with 25 mM Tris (pH8.0), 100 mM NaCl, 0.5% NP-40, 10 mM MgCl₂, 10% glycerol, lx protease inhibitor and PhosphoSTOP (Roche). Enrichment of target proteins was confirmed by immunoblot analysis. Complementary DNAs (cDNAs) were synthesized from TRIzol (Invitrogen)-isolated RNA, using Superscript III kits (Invitrogen). RNA-seq libraries were prepared and sequenced at Genome Services Laboratory, HudsonAlpha. Sequencing reads were mapped by Tophat and transcripts called by Cufflinks. Pulldown enrichment was calculated for each transcript as a ratio of FPKM recovered from TRAP-seq and RIP-seq samples compared to those from 5% input.

For RNA-seq analysis, volcano scores for wild-type, DDX5-T and RORγt knockout T_{H}17 cells were calculated for each transcript as a function of its P value and fold change between mutant and wild-type controls. BAM files were converted to .tdf format for viewing with the IGV Browser Tool. Ingenuity Pathway Analysis (IPA, Qiagen) was used to identify enriched Gene Ontology terms in the DDX5−RORγt coregulated gene set.

ChIRP-seq and ChIRP-qPCR. The ChIRP-seq assay was performed largely as described previously (58). Mouse T_{H}17 cells were cultured as above and in vivo RNA—chromatin interactions were fixed with 1% glutaraldehyde for 10 min at 25° C.

Antisense DNA probes (designated ‘odd’ or ‘even’) against Rmrp were designed by Biosearch Probe Designer [1,5′-TAGGAAACAGGCCTTCAGAG-3′ (SEQ ID NO: 8); 2,5′-AACATGTCCCTCGTATGTAG-3′ (SEQ ID NO: 9); 3,5′-CCCCTAGGCGAAAGGATAAG-3′ (SEQ ID NO: 10); 4,5′-AACAGTGACTTGCGGGGGAA-3′ (SEQ ID NO: 11); 5,5′-CTATGTGAGCTGACGGATGA-3′ (SEQ ID NO: 12)]. Probes modified with BiotinTEG at the 3′ end were synthesized by Integrated DNA Technologies (IDT). Isolated RNA was used in RT-qPCR analysis (Stratagene) to quantify enrichment of Rmrp and depletion of other cellular RNAs. Isolated DNA was used for qPCR analysis or to make deep sequencing libraries with the NEBNext DNA library prep master mix set for Illumina (NEB). Library DNA was quantified on the high sensitivity bioanalyzer (Agilent) and sequenced from a single end for 75 cycles on an Illumina NetSeq 500.

Sequencing reads were first trimmed of adaptors (FASTX Toolkit) and then mapped using Bowtie to a custom bowtie index containing single-copy loci of repetitive RNA elements (ribosomal RNAs, small nuclear RNAs, and noncoding Y RNAs (59). Reads that did not map to the custom index were then mapped to mm (9). Mapped reads were separately shifted towards the 3′ end using MACS and normalized to a total of 10 million reads. Even and odd replicates were merged as described previously (58) by taking the lower of the two read density values at each nucleotide across the entire genome. These processing steps take raw FASTQ files and yield processed files that contain genome-wide Rmrp-occupied chromatin association maps, where each nucleotide in the genome has a value that represents the relative binding level of the Rmrp RNA. MACS parameters were as follows: band width=300; model fold=10, 30; P-value cutoff=1×105. The full pipeline is available at https://github.com/bdo311/chirpseq-analysis.

ChIRP-qPCR was performed on DNA purified after treatment with RNase (60 min, 37° C.) and proteinase K (45 min, 65° C.). The primers used for qPCR are listed in Supplementary Table 1. For enrichment analysis, the present inventors tested for the enrichment of Rmrp ChIRP peaks among ChIP peak sets for key T_{H}17 transcription factors, CTCF, RNA Pol II and several histone marks (8). Assay for transposase-accessible chromatin sequencing (ATAC)-seq was performed, according to published protocols (60), on cultured T_{H}17-polarized cells in vitro for 48 h. Because of differences in ChIP antibody affinities and the bias in the selection of ChIP and ChIRP factors, the present inventors used peaks generated from ATAC-seq data as a background setting for the enrichment analysis. In this analysis, the present inventors considered all ChIRP and ChIP peaks that fell within ±500 base pairs of ATAC-seq peaks, and then calculated the overlap among the ChIRP and ChIP sets, using the hypergeometric distribution to estimate significance.

In vitro binding assay. For in vitro binding assays, pcDNA3.1-Rmrp vectors were used for T7 polymerase-driven in vitro transcription (IVT) reactions (Promega). Haemagglutinin (HA)−DDX5 and Flag−RORγt were in vitro transcribed and translated using an in vitro transcription and translation (TNT) system according to themanufacturer's protocol (Promega). Alternatively, pGEX4.1-DDX5 (wild-type and helicase-dead mutant) constructs were transformed into BL21 to synthesize recombinant full-length GST−hDDX5 proteins. Full-length His-tagged human RORγt was purified in three steps through Ni-resin, S column and gel-filtration (AKTA). Then, 0.5 μg of each recombinant protein was incubated in the presence or absence of 200 μM ATP, 300 ng in vitro transcribed Rmrp in co-immunoprecipitation buffer containing 25 mM Tris (pH8.0), 100 mM NaCl, 0.5% NP-40, 10 mM MgCl₂, 10% glycerol, lx protease inhibitor, RNaseInhibitor (Invitrogen) and PhosphoSTOP (Roche). GST−DDX5 was enriched on glutathione beads (GE); HA−DDX5, Flag−RORγt and His−RORγt were enriched using anti-HA (Covance), anti-Flag (Sigma) and anti-His antibodies (Santa Cruz Bio) coupled to anti-mouse immunoglobulin dynabeads (Dynal, Invitrogen).

Microscopy. T_{H}17 cells were cultured on glass coverslips for 48 h and fixed in 4% paraformaldehyde in PBS for 5 min at room temperature. Fixed cells were permeabilized with 0.1% bovine serum albumin (BSA), 0.1% Triton and 10% normal serum in PBS for 1 h. Cells were then incubated with primary antibodies (DDX5(Abcam) or RORγt (eBiosciences)) in 0.1% BSA and 0.2% Triton PBS overnight at 4° C. Secondary antibodies (anti-goat Alexa 488 or anti-rat Alexa 647 (Molecular Probe)) were incubated at 4° C. for 1 h. Stained cells were washed three times with 0.5% Tween and 0.1% BSA in PBS. DAPI was used to stain DNA inside the nucleus. Immunofluorescence images were captured on a Zeiss 510 microscope at 40×.

ChIP and RT-qPCR analysis. T_{H}17-polarized cells were crosslinked with 1% paraformaldehyde (EMS) and incubated with rotation at room temperature. Crosslinking was stopped after 10 min with glycine to a final concentration of 0.125 M and incubated for a further 5 min with rotation. Cells were washed with 3× ice-cold PBS and pellets were either flash-frozen in liquid N₂ or immediately resuspended in Farnham lysis buffer (5 mM PIPES, 85 mM KCl, 0.5% NP-40). Hypotonic lysis continued for 10 min on ice before cells were spun down and resuspended in RIPA buffer (lx PBS, 1% NP-40, 0.5% SDS, 0.5% Na-deoxycholate), transferred into TPX microtubes and lysed on ice for 30 min. Nuclear lysates were sonicated for 40 cycles of 30 s ‘ON’ and 30 s ‘OFF’ in 10-cycle increments using a Biorupter (Diadenode) set on high. After pelleting debris, chromatin was precleared with protein G dynabeads (dynabeads, TFS) for 2 h with rotation at 4° C. For immunoprecipitation, precleared chromatin was incubated with anti-RORγt antibodies (1 μg per 2×10⁶ cells) overnight with rotation at 4° C. and protein G was added for the final 2 h of incubation. Beads were washed and bound chromatin was eluted. ChIP-qPCR was performed on DNA purified after treatment with RNase (30 min, 37° C.) and proteinase K (2 h, 55° C.) followed by reversal of crosslinks (8-12 h, 65° C.). The primers used for qPCR have been described previously (5).

For analysis of mRNA transcripts, gene specific values were normalized to the Gapdh housekeeping gene for each sample. All primer sequences are listed in Supplementary Table 1.

References Relating to Materials and Methods
47. Stanley, S. et al. Profiling of glucose-sensing neurons reveals that GHRH neurons are activated by hypoglycemia. Cell Metab. 18, 596-607 (2013).
48. Nicol, S. M. et al. The RNA helicase p68 (DDX5) is selectively required for the induction of p53-dependent p21 expression and cell-cycle arrest after DNA damage. Oncogene 32, 3461-3469 (2013).
49. Croswell, A., Amir, E., Teggatz, P., Barman, M. & Salzman, N. H. Prolonged impact of antibiotics on intestinal microbial ecology and susceptibility to enteric Salmonella infection. Infect. Immun. 77, 2741-2753 (2009).
50. Ostanin, D. V. et al. T cell transfer model of chronic colitis: concepts, considerations, and tricks of the trade. Am. J. Physiol. Gastrointest. Liver Physiol.296, G135-G146 (2009).
51. Kim, S. V. et al. GPR15-mediated homing controls immune homeostasis in the large intestine mucosa. Science 340, 1456-1459 (2013).
52. Manel, N., Unutmaz, D. & Littman, D. R. The differentiation of human TH-17 cells requires transforming growth factor-β and induction of the nuclear receptor RORgammat. Nature Immunol. 9, 641-649 (2008).
53. Meng, L. et al. Towards a therapy for Angelman syndrome by targeting a long non-coding RNA. Nature 518, 409-412 (2015).
54. Bates, G. J. et al. The DEAD box protein p68: a novel transcriptional coactivator of the p53 tumour suppressor. EMBO 1 24, 543-553 (2005).
55. Morita, S., Kojima, T. & Kitamura, T. Plat-E: an efficient and stable system for transient packaging of retroviruses. Gene Ther. 7, 1063-1066 (2000).
56. Santori, F. R. et al. Identification of natural RORy ligands that regulate the development of lymphoid cells. Cell Metab. 21, 286-297 (2015).
57. Huh, J. R. et al. Identification of potent and selective diphenylpropanamide RORgamma Inhibitors. ACS Med. Chem. Lett. 4, 79-84, (2013).
58. Chu, C., Qu, K., Zhong, F. L., Artandi, S. E. & Chang, H. Y. Genomic maps of long noncoding RNA occupancy reveal principles of RNA-chromatin interactions. Mol. Cell 44, 667-678 (2011).
59. Flynn, R. A. et al. Dissecting noncoding and pathogen RNA-protein interactomes. RNA 21, 135-143 (2015).
60. Buenrostro, J. D., Wu, B., Chang, H. Y. & Greenleaf, W. J. ATAC-seq: a method for assaying chromatin accessibility genome-wide. Curr. Protoc. Mol. Biol. 109,21.29.1-21.29.9. (2015).

### Results

### DDX5 Regulation of RORγt Target Genes

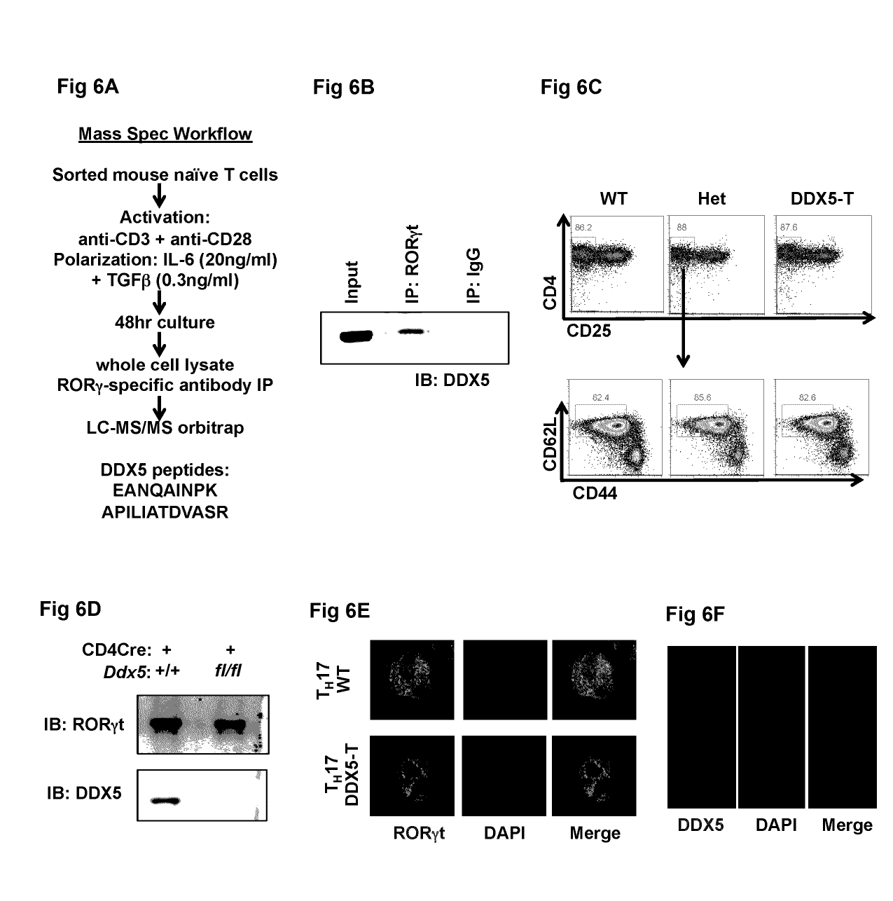

To identify novel interacting partners of RORγt in T_{H}17 cells, the present inventors enriched for endogenous RORγt-containing protein complexes and subsequently determined protein composition using liquid-chromatography-tandem mass spectroscopy (LC-MS/MS) (workflow shown in FIG. 6a). Among the top hits of RORγt-interacting proteins was the RNA helicase DDX5. This interaction was validated through conventional co-immunoprecipitation experiments followed by immunoblot analysis (FIG. 6b).

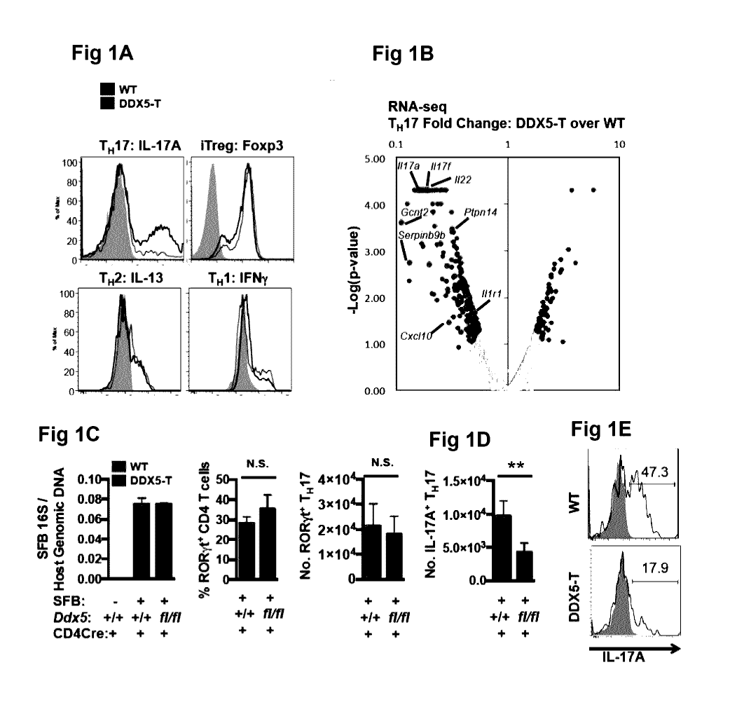

The present inventors investigated the function of DDX5 in T cells by breeding Ddx5 conditional mutant mice with CD4-Cre mice to generate T-cell-specific DDX5-deficient mice (Dcbc5fl/fl CD4-Cre mice, denoted DDX5-T). DDX5-T mice were born at the expected Mendelian ratio, were fertile, and did not display any gross phenotypic abnormalities. The activation status of T cells in the periphery was similar between Dcbc5+/+CD4-Cre+ (wild type) and mutant mice (FIG. 6c) that had no DDX5 protein in spleen and lymph node CD4+ T cells (FIG. 6d). Naive CD4+ T cells sorted from wild-type and DDX5-T mice did not display differences in polarization towards TH1, TH2 and induced regulatory T (iTreg) cell phenotypes in vitro (FIG. 1a). In contrast, DDX5-T naive T cells cultured under T_{H}17-polarizing conditions produced substantially less IL-17A than wildtype cells (FIG. 1a). RORγt protein expression and nuclear localization were similar between wild-type and DDX5-T T_{H}17-polarized cells (FIG. 6d, e), and, like RORγt, DDX5 protein localized mainly to the nucleus (FIG. 6f). These results suggest that DDX5 is not required for T_{H}17 lineage commitment, but contributes to T_{H}17 cell effector functions.

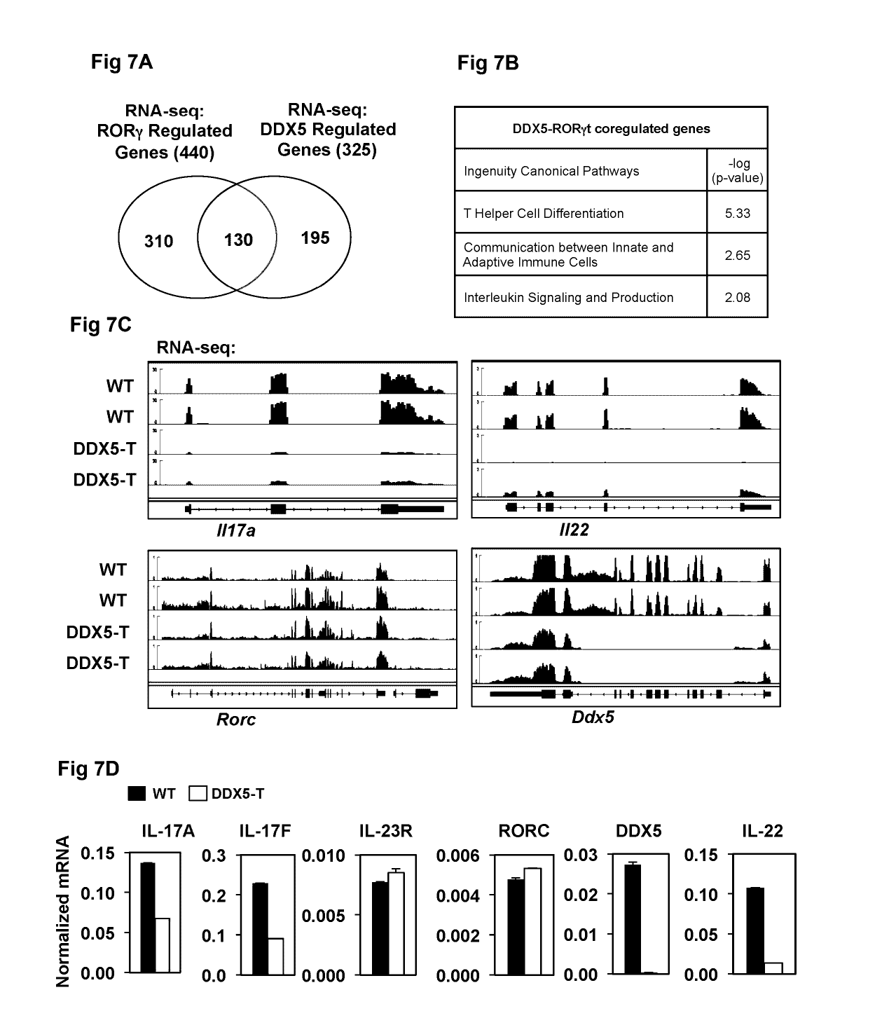

DDX5 can function as a transcriptional coactivator (12, 24, 25), augmenting the activities of other nuclear receptor family members, including the oestrogen and androgen receptors (12, 26). To determine whether DDX5 partners with RORγt to facilitate the T_{H}17 cell transcriptional program, the present inventors performed RNA sequencing (RNA-seq) on in vitro polarized T_{H}17 cells from wild-type or DDX5-T mice. Among the 325 genes that were significantly dysregulated in DDX5-deficient T cells 96 h after polarization, approximately 40% had been previously identified as RORγt targets in T_{H}17 cells (8) (FIG. 7a). Ingenuity Pathway Analysis (Qiagen) of DDX5−RORγt-coregulated genes revealed enrichment in ‘T helper cell differentiation program’ as well as ‘interleukin production’ (FIG. 7b). Coregulated genes (FIG. 1b) included those for the signature T_{H}17 cytokines (Il17a,Il17f and Il22) (FIG. 7c). Independent biological samples were used to validate a subset of RORγt target genes with and without altered expression in DDX5-deficient T_{H}17 cells (FIG. 7d).

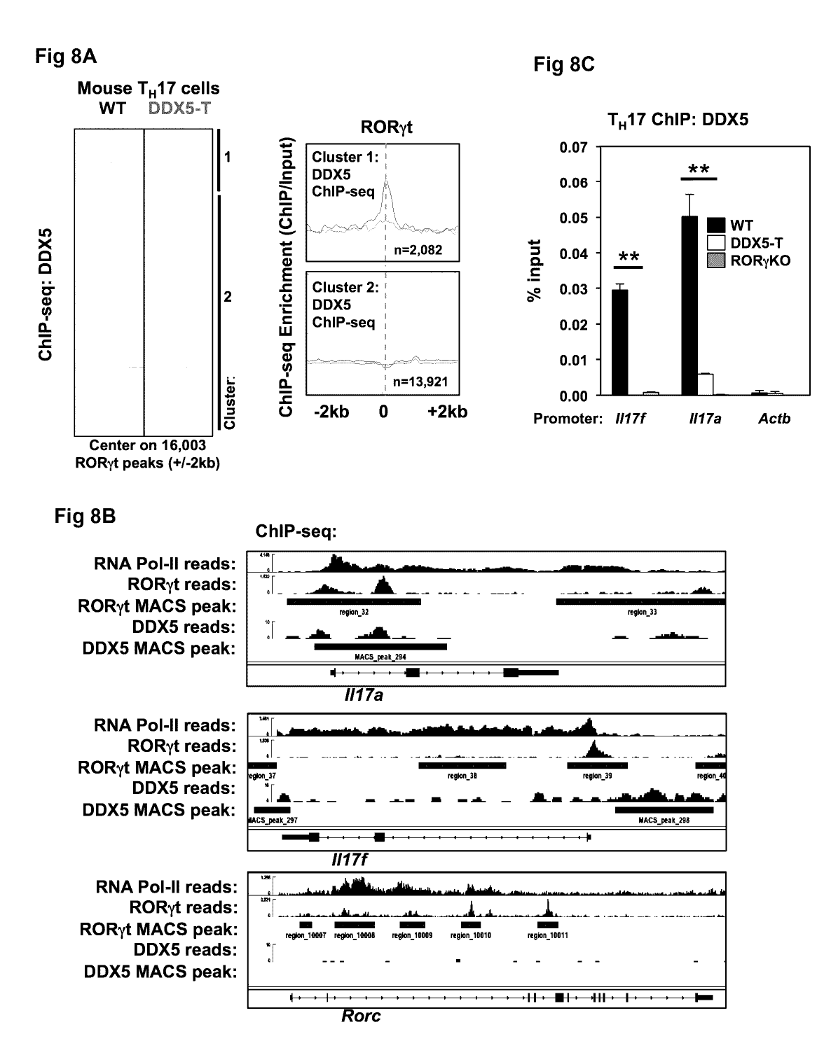

We used anti-DDX5 antibodies in genome-wide chromatin immunoprecipitation sequencing (ChIP-seq) studies to identify DDX5-occupied loci. A specific subset of previously published RORγt-occupied loci, including Il17a and Il17f were enriched for DDX5 co-localization, as determined by seqMINER clustering analysis (FIG. 8a, b). ChIP with quantitative PCR (ChIP-qPCR) was used to validate DDX5 enrichment at the Il17a and Il17f loci and its dependency on RORγt in polarized T_{H}17 cells (FIG. 8c). These results suggest that DDX5 overlaps with RORγt in modulating a specific subset of the T_{H}17 cell transcriptional program.

### DDX5 Function In Vivo in T_{H}17 Cells

At steady state, cytokine-producing T_{H}17 cells populate the small intestinal lamina propria of animals colonized with commensal segmented filamentous bacteria (SFB) (27). When colonized with SFB, DDX5-T mice and wild-type littermates had similar numbers of ileal-residing Foxp3−RORγt+CD4+ T_{H}17 cells (FIG. 1c). However, the number and proportion of IL-17A-producing cells among RORγt+CD4+ cells from DDX5-T mice were markedly reduced compared to wild-type littermate controls (FIG. 1d, e).

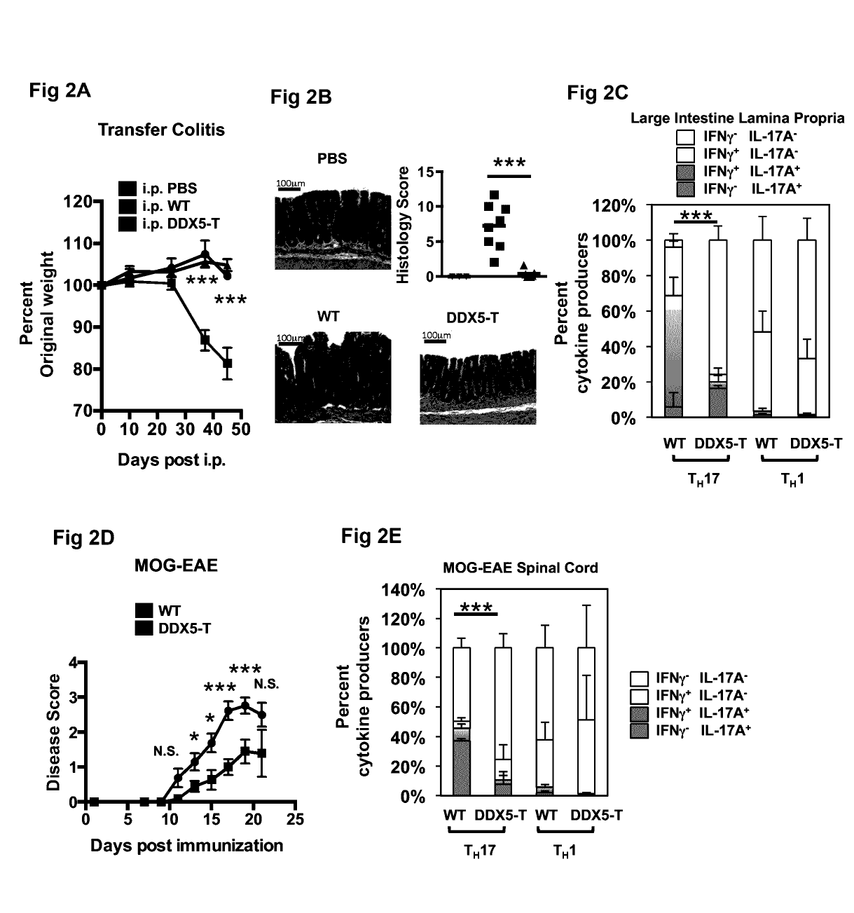

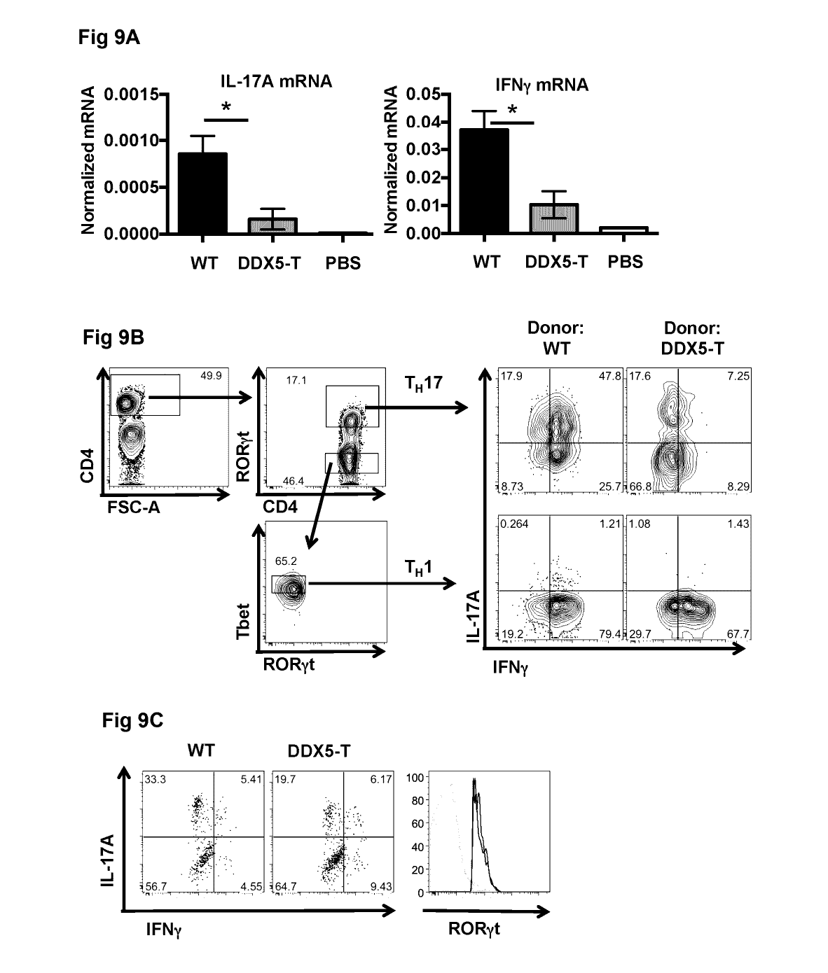

To evaluate the role of DDX5 in T_{H}17-driven inflammation, the present inventors used a T-cell transfer model of colitis, in which disease severity is dependent on RORγt expression in donor T cells (3,28). Following transfer of CD4+CD45RBhi T cells into Rag-deficient (Rag2−/−) recipients, mice that received wild-type T cells experienced weight loss (FIG. 2a) and developed colitis (FIG. 2b), whereas recipients of DDX5-T cells did not. Total RNA from large intestine lamina propria mononuclear cells revealed a significant reduction of both Il17a and lfng transcripts from recipients of DDX5-T cells compared to wild-type controls (FIG. 9a). Interestingly, there were comparable proportions of IFNγ-producing CD4+RORγt−T-bet+ (conventional TH1) cells in recipients of T cells from either wild-type or DDX5-T mice (FIG. 9b). However, recipients of cells from DDX5-T mice displayed a significant reduction in CD4+ Foxp3−RORγt+ T cells coexpressing IL-17A and IFNγ, an important feature of pathogenic T cells in several inflammatory disease settings (2,29,30) (FIG. 2c and FIG. 9b). Consistent with a loss of pathogenic capacity, DDX5-T mice also exhibited attenuated disease compared to wild-type controls during EAE (FIG. 2d). Analysis of spinal cord infiltrates after immunization revealed a reduced proportion of IL-17A-producing CD4+ T cells (FIG. 2e and FIG. 9c). Consistent with the present inventors' in vitro findings, these results in mice indicate that DDX5 selectively regulates the T_{H}17 effector program, both in steady state and under inflammatory conditions.

### Function of DDX5-Associated LncRNA

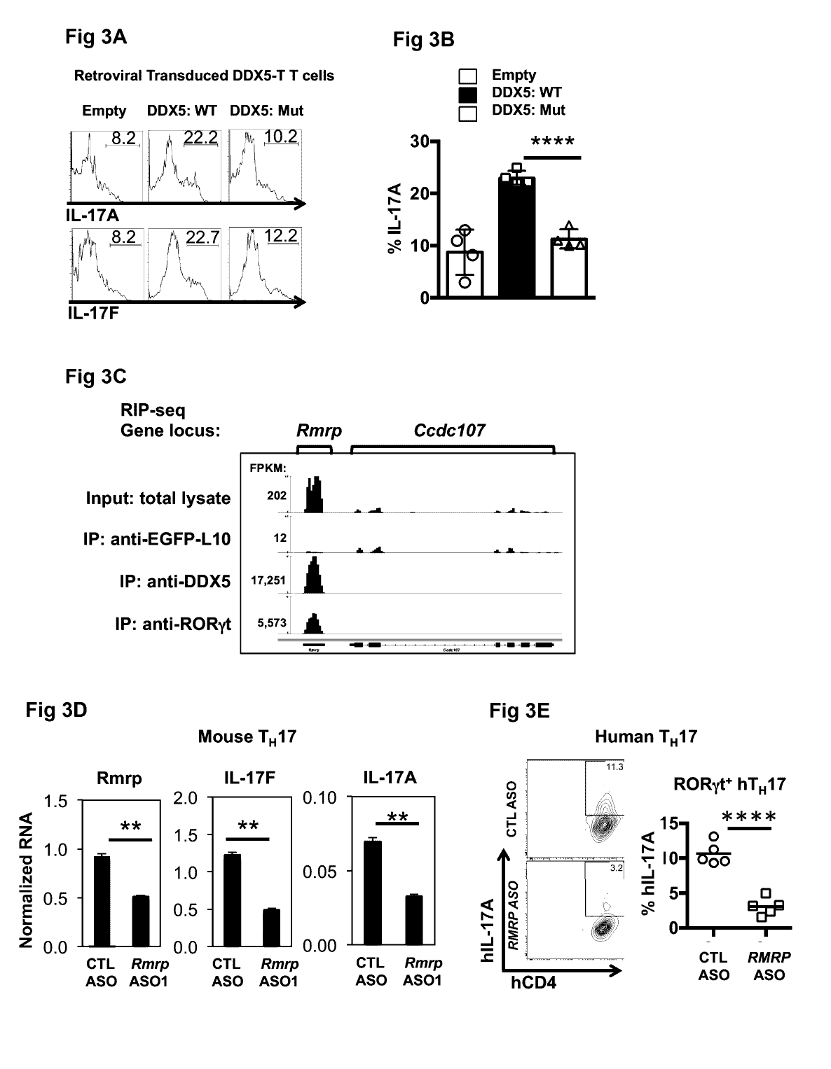

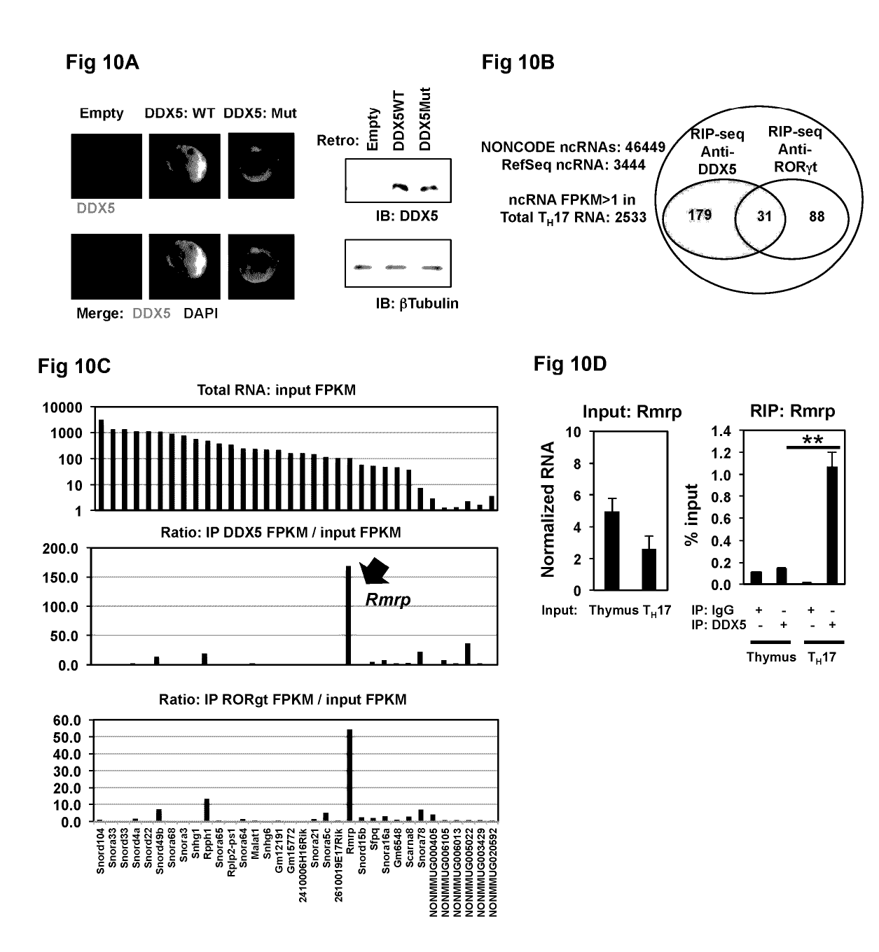

RNA helicases are highly conserved enzymes that utilize the energy derived from ATP hydrolysis to unwind RNA duplexes, facilitate RNA annealing, and displace proteins from RNA. It was previously shown that DDX5 transcriptional coactivator activity for oestrogen receptor, androgen receptor and the transcription factor RUNX2 is independent of RNA helicase activity (12, 24, 26). The present inventors tested this requirement in the context of RORγt by retroviral transduction of DDX5-deficient T cells cultured under T_{H}17-polarizing conditions with expression constructs for wild-type or mutant DDX5 with an inactivated helicase domain (helicase-dead). Surprisingly, only wild-type DDX5 rescued IL-17A and IL-17F production in these polarized T_{H}17 cells (FIG. 3a, b and FIG. 10a). This result suggested that perhaps RNA substrate(s) for the helicase activity of DDX5 contribute to its transcriptional coactivator role in T_{H}17 cells.

The present inventors next searched for RNA molecules that might participate in DDX5−RORγt-mediated transcription in T_{H}17 cells. The present inventors first depleted ribosome-bound mRNAs undergoing active protein synthesis. Lysates pre-cleared of ribosomes were then subjected to RNA immunoprecipitation (RIP) with antibodies specific for DDX5 or RORγt, followed by deep sequencing of the associated RNAs (RIP-seq). Among 49,893 annotated lncRNAs in the mouse RefSeq and NONCODE database, 2,533 noncoding RNAs were expressed in T_{H}17 cells (fragments per kilobase of transcripts per million mapped reads (FPKM) >1, FIG. 10b). Interestingly, the steroid receptor RNA activator (SRA) lncRNA, previously found to be associated with DDX5 in muscle cells (15), was not enriched in DDX5-containing protein complexes in T_{H}17 cells. Instead, the present inventors found Rmrp to be the most enriched RNA associated with DDX5 and, to a lesser degree, RORγt, in T_{H}17 cells (FIG. 3c and FIG. 10c). RIP-qPCR with independent biological samples confirmed enrichment of Rmrp RNA in DDX5 pull-downs from T_{H}17 cells, but not from thymocyte lysates (FIG. 10d).

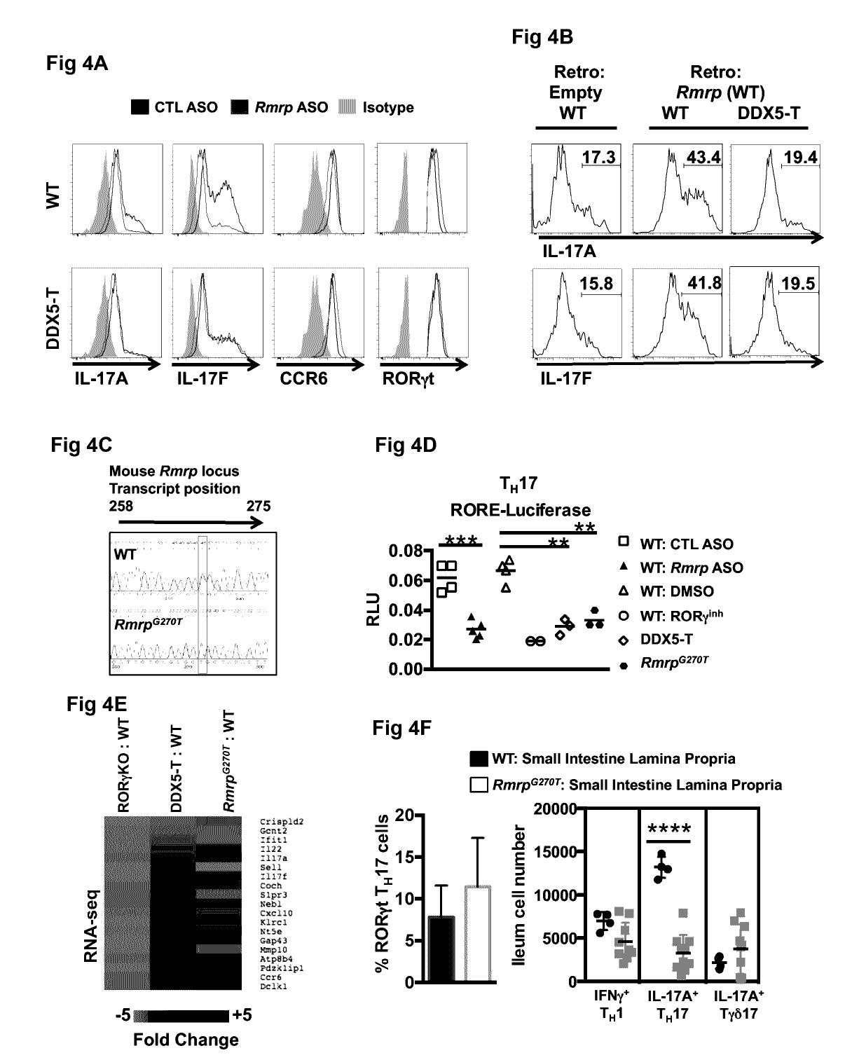

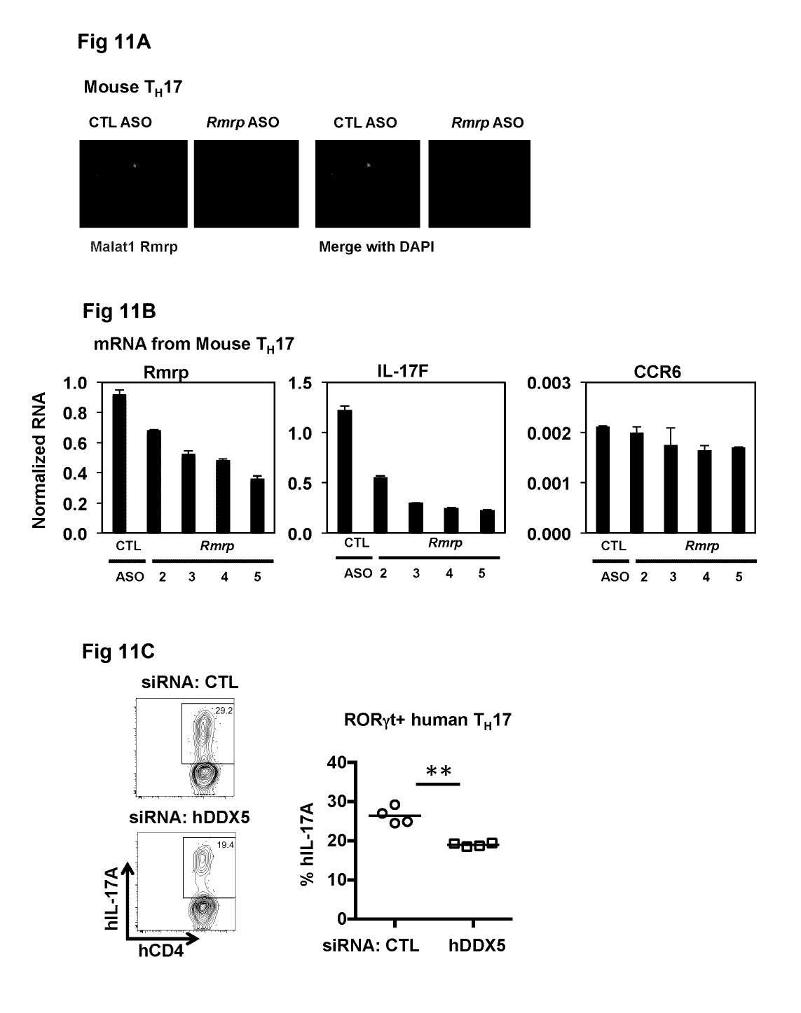

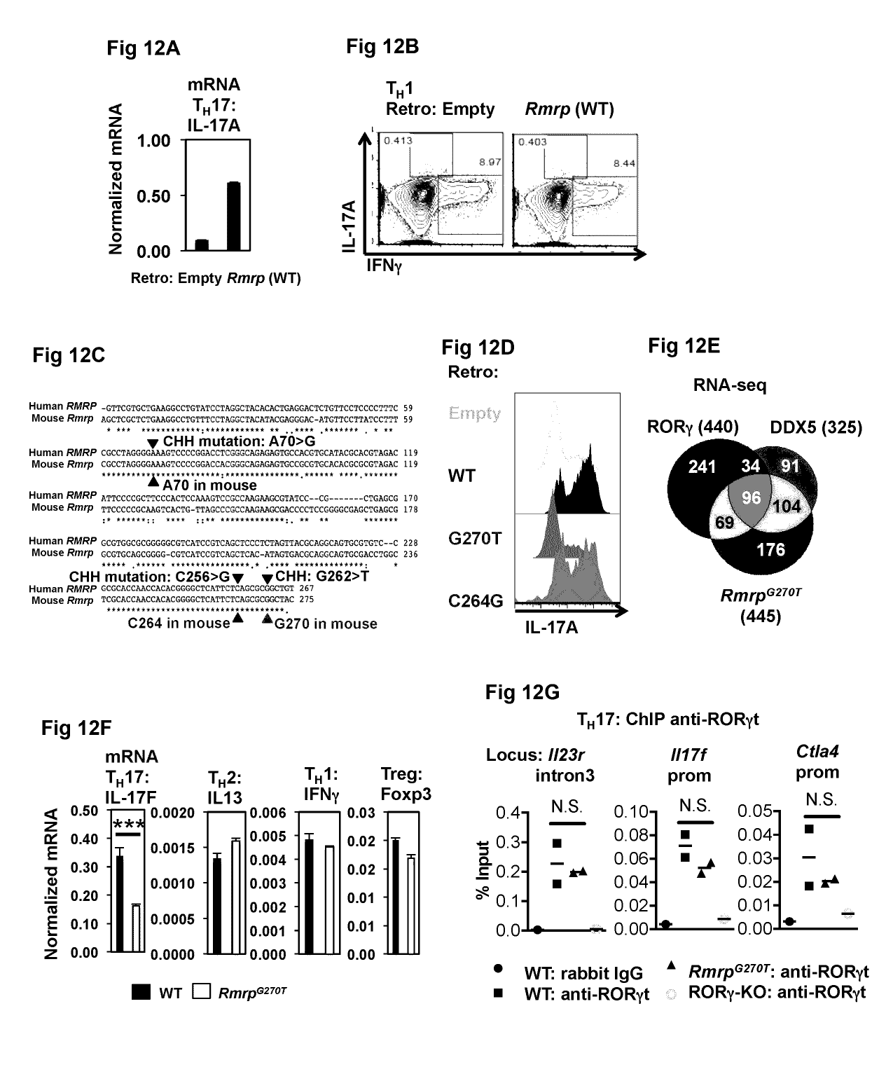

RNA fluorescence in situ hybridization revealed that Rmrp is localized in the nucleus of T_{H}17 cells (FIG. 11a). To evaluate the functional role of Rmrp, the present inventors transiently depleted Rmrp RNA from primary mouse T_{H}17 cells using an RNaseH-dependent antisense oligonucleotide (ASO). Similar to the DDX5-deficient T_{H}17 cells, cells depleted of Rmrp expressed reduced Il17a and Il17fmRNA (FIG. 3d and FIG. 11b). Human T_{H}17 cells also displayed reduced cytokine production upon depletion of RMRP or DDX5 (FIG. 3e and FIG. 11c), suggesting that this regulatory mechanism is evolutionarily conserved. Notably, Rmrp RNA knockdown in DDX5-deficient mouse T_{H}17 cells did not further reduce IL-17A and IL-17F expression (FIG. 4a). Expression of RORγt-dependent, but DDX5-independent, CCR6 was unaffected by the reduction in Rmrp. Transduction of Rmrp into T cells cultured in TH1-polarization conditions had little effect on IFNγ production, but there was marked enhancement of IL-17A and IL-17F production in wild-type, but not DDX5-deficient, cells cultured in T_{H}17-polarization conditions (FIG. 4b and FIG. 12a, b). Thus, Rmrp-dependent cytokine gene expression requires the presence of DDX5.

### T_{H}17 Program in Rmrp Mutant Mice

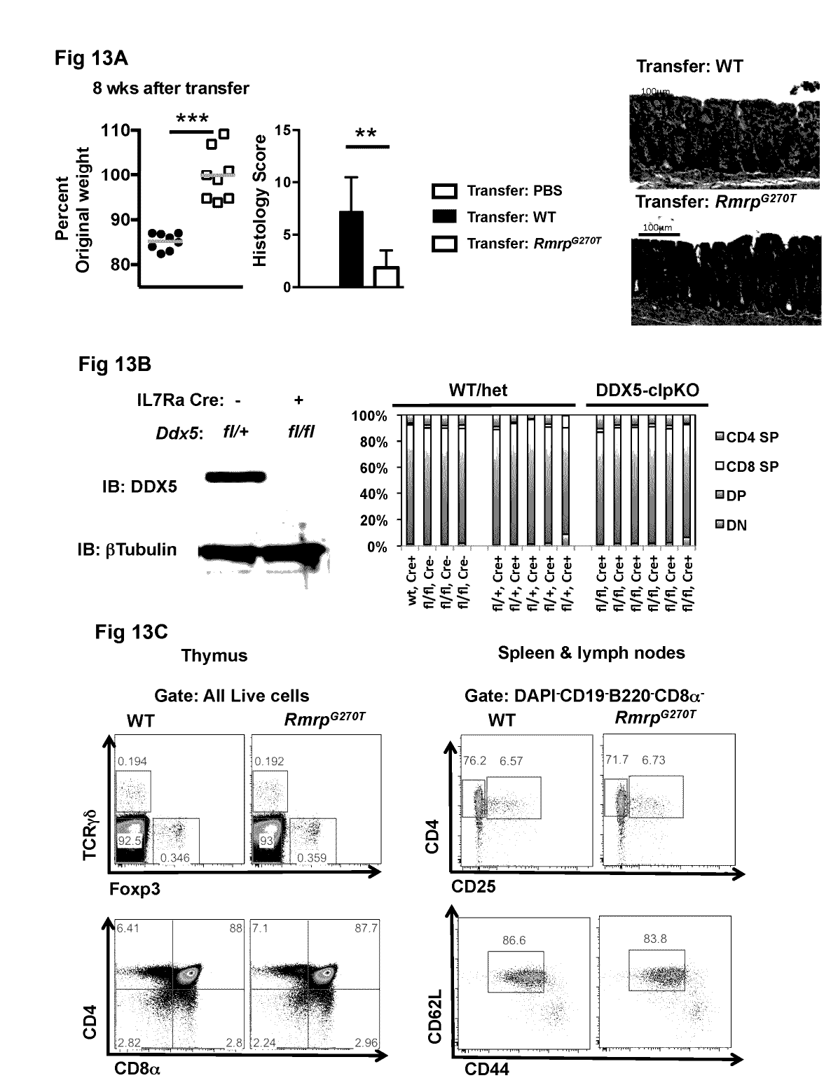

In contrast to wild-type Rmrp, a mutant Rmrp carrying a single nucleotide change (270G >T), corresponding to an allele identified in CHH patients (262G >T), failed to potentiate IL-17A production after transduction into T_{H}17-polarized cells (FIG. 12c, d). To investigate whether G270 of Rmrp contributes to RORγt transcriptional output in vivo, the present inventors generated mice homozygous for the Rmrp G270T point mutation, using CRISPR-Cas9 (clustered, regularly interspaced short palindromic repeats coupled with CRISPR-associated proteins) technology (FIG. 4c). The mice were born at the expected Mendelian ratios and had no gross defects. ROR response element (RORE)-regulated luciferase activity was reduced in transiently transfected T_{H}17 cells from DDX5-deficient and RmrpG270T mice and after ASO-mediated knockdown of Rmrp (FIG. 4d). Comparison of the transcription profiles of in vitro polarized T_{H}17 cells from wild-type, RORγt-deficient, DDX5-deficient and RmrpG270T/G270T mice indicated that 96 RORγt-dependent T_{H}17 cell genes were coregulated by Rmrp together with DDX5 (FIG. 12e and FIG. 4e). Reverse transcription (RT)-qPCR analysis of independent biological samples from in vitro polarized T cells from wild-type and RmrpG270T/G270T mice confirmed reduced Il17f mRNA expression in the latter (FIG. 12f), despite a similar amount of RORγt binding to known cis-regulatory loci (FIG. 12g). The proportion of RORγt+Foxp3−T_{H}17 cells among total ileal lamina propria CD4-lineage cells was unaffected in RmrpG270T/G270T mice, but these cells expressed relatively little IL-17A compared to those in wild-type littermates (FIG. 4f). Transfer of RmrpG270T/G270T T cells into Rag2−/− mice resulted in reduced colitis, as determined by weight loss and colon histology, compared to the transfer of wild-type cells (FIG. 13a). These phenotypes are similar to those observed in recipients of DDX5-deficient T-cells (FIG. 2a,c), which is consistent with an important role of Rmrp G270 in executing the T_{H}17 effector program in vivo.

RORγt and its closely related isoform RORγ perform distinct functions in diverse tissues. RORγt is critical for thymocyte development, regulating the survival of double-positive CD4+CD8+ cells, and development of secondary and tertiary lymphoid organs mediated by lymphoid tissue inducer cells (31). While DDX5 and Rmrp are ubiquitously expressed, Rmrp was less enriched in thymocyte-derived than in T_{H}17-cell-derived DDX5 immunoprecipitates (FIG. 10d). When Ddx5 was inactivated at the common lymphoid progenitor stage, by breeding the conditional mutant mice with IL7R-Cre mice, there was no apparent defect in thymocyte subset development (FIG. 13b). Similarly, RmrpG270T knock-in mice displayed normal thymocyte subsets and also had intact secondary lymphoid organ development (FIG. 13c). Together, these results suggest that the DDX5−Rmrp complex performs T_{H}17-specific functions.

### Rmrp in DDX5−RORγt Complex Formation

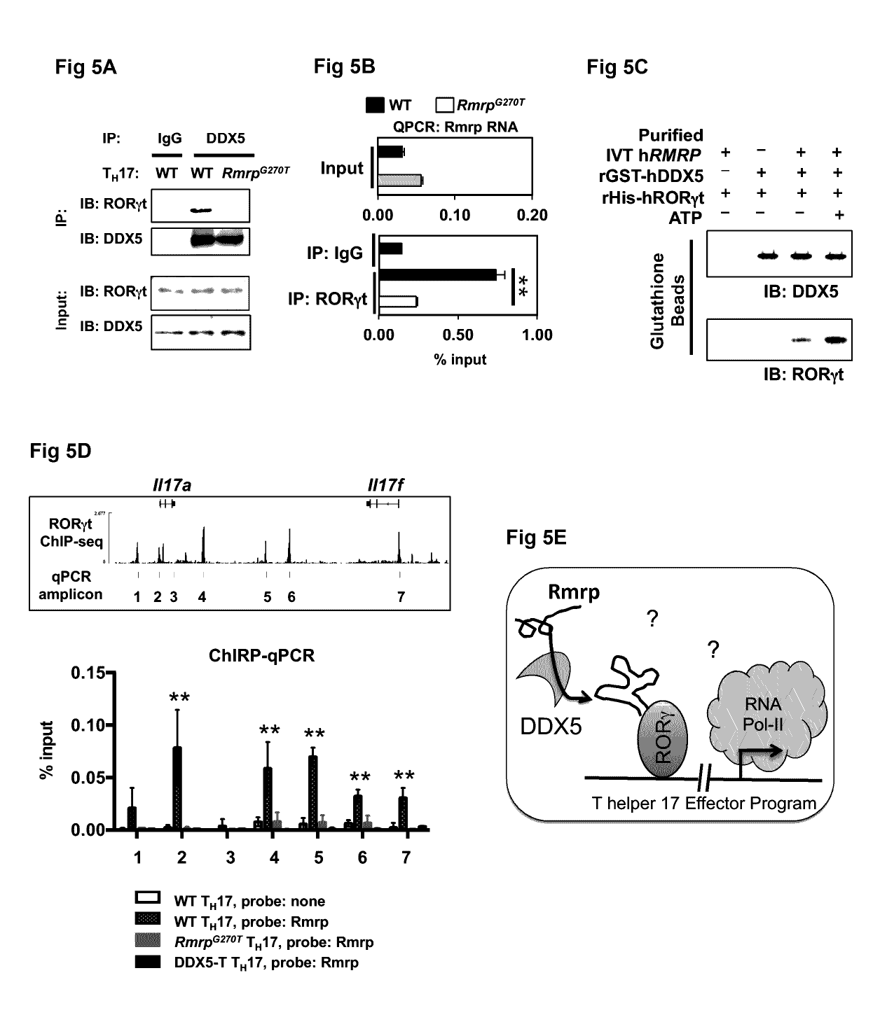

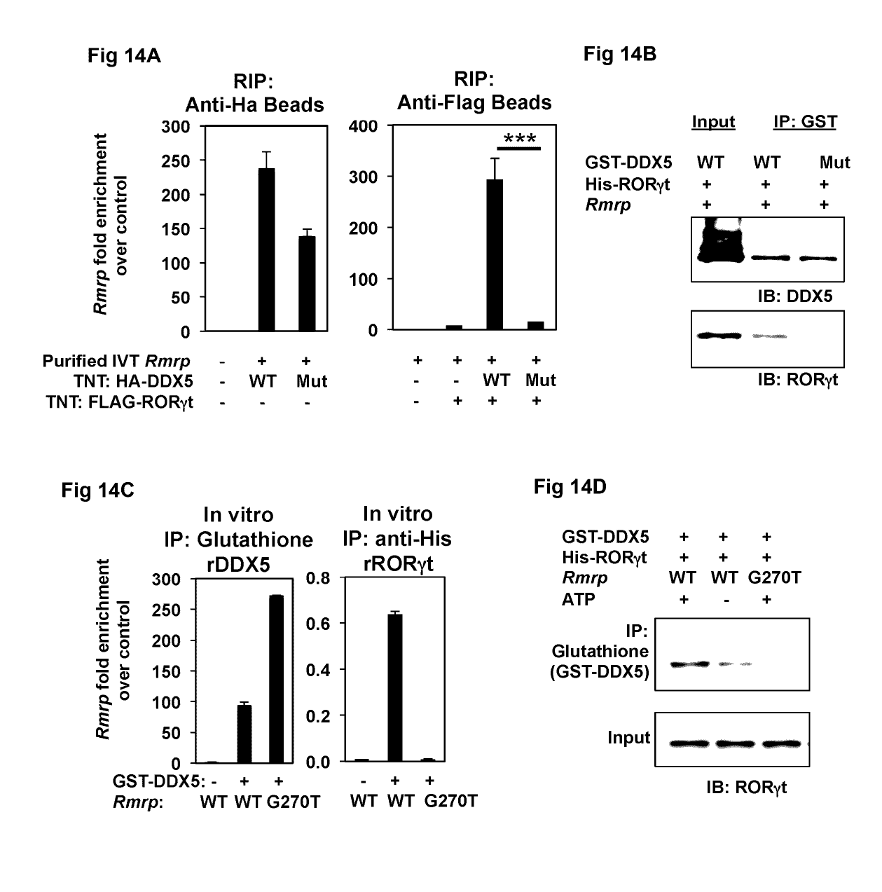

The present inventors next investigated how Rmrp contributes to the DDX5−RORγt-regulated transcriptional circuit in T_{H}17 cells. RORγt−DDX5 complex assembly was severely compromised in T_{H}17 cells from RmrpG270T mice (FIG. 5a). Moreover, Rmrp recruitment to the RORγt protein complex was significantly reduced in T_{H}17 cells from Rmrp mutant animals (FIG. 5b). In vitro, Rmrp binds directly to recombinant DDX5 (FIG. 14a). Notably, Rmrp was recruited to RORγt in the presence of wild-type, but not helicase-dead, DDX5. Furthermore, in vitro transcribed Rmrp RNA promoted RORγt interaction with wild-type, but not helicase-dead, DDX5 in the presence of ATP (FIG. 5c and FIG. 14b). Mutant Rmrp was also defective in mediating DDX5−RORγt complex assembly in vitro (FIG. 14c, d).

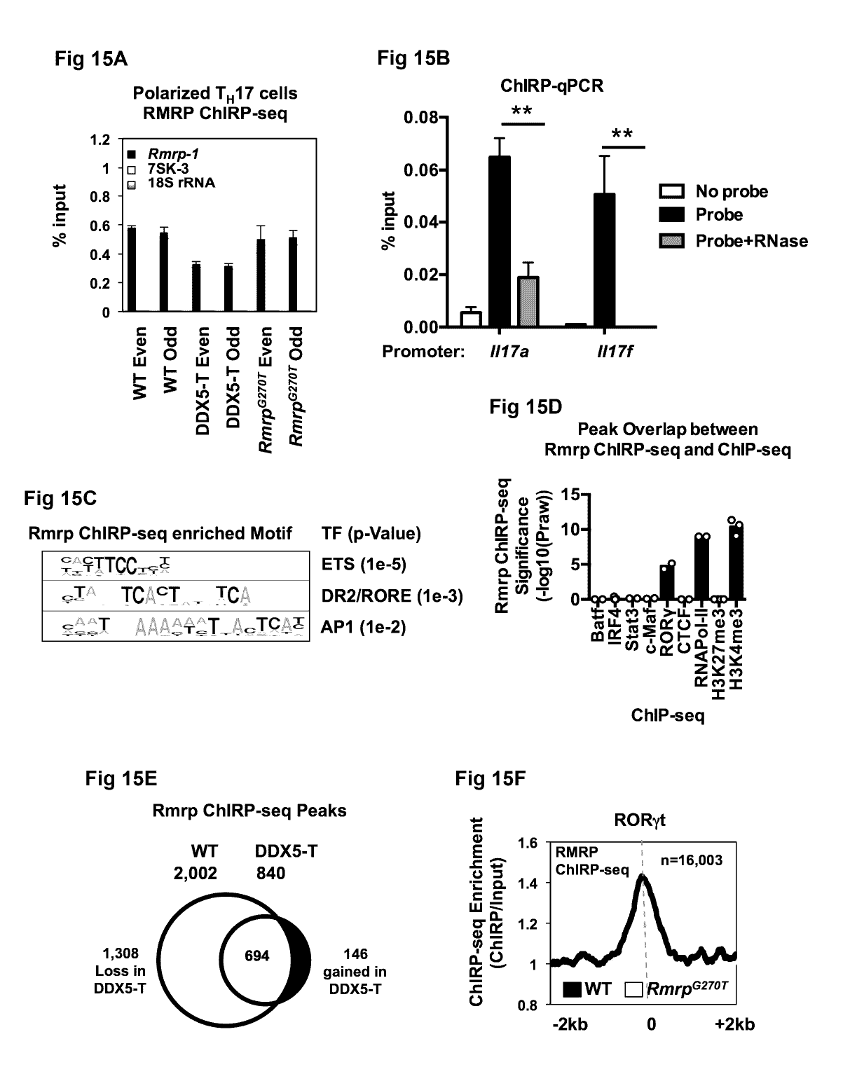

To determine whether Rmrp is associated with specific genomic loci, the present inventors performed chromatin isolation by RNA purification (ChIRP) followed by either locus-specific qPCR or deep sequencing (ChIRPseq) (32). The present inventors used two orthogonal antisense probe sets that specifically and robustly recovered Rmrp from T_{H}17 cells (FIG. 15a). When combined for Rmrp ChIRP-qPCR, the probes recovered RORγt-bound regions in the Il17a and Il17f loci from T_{H}17-polarized cells of wild-type but not DDX5-T or RmrpG270T/G270T mice, in an RNase-sensitive manner (FIG. 5d and FIG. 1b). For ChIRP-seq, the present inventors focused their analysis on signals that overlapped with separate use of the two probe sets. HOMER motif analyses of Rmrp peak regions identified the ETS, DR2/RORE and AP1 transcription factor motifs to be the most highly enriched (FIG. 15c). Consistent with this, Rmrp ChIRP-seq significantly overlapped with RORγt-bound loci, but not with sites occupied by CTCF or by other T_{H}17 transcription factors, such as BATF, IRF4, STAT3 and c-Maf (FIG. 15d). There was also significant overlap with RNA polymerase II (Pol II)- and histone H3 lysine 4 trimethylation (H3K4me3)-associated chromatin, which mark actively transcribed regions. Concordantly, ChIRP-seq of Rmrp in DDX5−T T_{H}17 cells revealed a loss of called Rmrp peaks (FIG. 15e), consistent with a DDX5 contribution to Rmrp association with chromatin. Rmrp association with RORγt-bound sites was also reduced in polarized T_{H}17 cells from RmrpG270T/G270T mice despite a similar amount of RNA recovery (FIG. 15f). Together, these results indicate that G270 of Rmrp is critical for DDX5−RORγt complex assembly and Rmrp recruitment to RORγt-occupied loci to coordinate the T_{H}17 effector program in trans.

### Discussion

Nuclear lncRNAs have key roles in numerous biological processes (33) including adaptive and innate immunity (34, 35), but how individual lncRNAs perform their activities and whether they contribute to immunological diseases remain unknown. The present inventors identified nuclear Rmrp as a key DDX5-associated RNA required to promote assembly and regulate the function of RORγt transcriptional complexes at a subset of critical genes implicated specifically in the T_{H}17 effector program (model in FIG. 5e). Rmrp thus acts in trans on multiple RORγt-dependent genes, and does so only upon interaction with enzymatically active DDX5 helicase. RNA-helicase-dependent functions of lncRNAs have been described, for example, the Drosophila male cell-specific lncRNAs roX1 and roX2 that are modified by the MLE helicase to enable expression of X-chromosome genes (36, 37). In addition, DDX21 helicase activity in HEK293 cells is required for 7SK RNA regulation of polymerase pausing at ribosomal genes (38). Results presented herein extend the concept of RNA helicase/1ncRNA function to lineage-specific regulation of transcriptional programs.

Notably, unlike most lncRNAs, Rmrp is highly conserved among mammals. In humans, mutations of evolutionarily conserved nucleotides at the promoter or within the transcribed region of RMRP result in CHH (21, 22). T cells from mice carrying a single nucleotide change (270G>T) in Rmrp, corresponding to one found in CHH patients (262G>T), had a compromised T_{H}17 cell effector program. CHH patients have been noted to have defective T-cell-dependent immunity, which may in part reflect reduced RA/RP-dependent activity at RORγt target genes. As forced expression of either DDX5 or Rmrp-enhanced T_{H}17 cytokine production, it is also possible that gain-of-function mutations in either of these molecules may contribute to T_{H}17-dependent inflammatory diseases.

RORγt is an attractive therapeutic target for multiple autoimmune diseases (5, 39). However, RORγt and RORγ have several other functions that would probably be affected by targeting of their shared ligand-binding pocket. RORγt is required for the development of early thymocytes, lymphoid tissue inducer cells that initiate lymphoid organogenesis (31), type 3 innate lymphoid cells that produce IL-22 and protect epithelial barriers, and for IL-17 production by ‘innate-like’ T cells, including T cell receptor (TCR)γδ and natural killer T cells (40-43). In the liver, RORγ contributes to regulation of metabolic functions (44). Mechanisms by which RORγt and RORγ differentially regulate transcription in these diverse cell types remain poorly understood. DDX5 and Rmrp are abundantly expressed in developing T cells in the thymus and in peripheral naive and differentiated T-helper subsets. Notably, the contribution of DDX5−Rmrp to RORγt-dependent functions appears to be confined to T_{H}17 cells, as their loss of function did not affect thymocyte or lymphoid organ development. Results presented herein raise the prospect that tissue- or cell-type-specific mechanisms exist to regulate how RNA helicases and their associated lncRNAs are assembled with distinct transcriptional complexes to promote diverse gene expression programs.

We speculate that the function of DDX5−Rmrp may be induced in response to specific tissue microenvironments in vivo. T_{H}17 cells differentiate at mucosal barriers in response to signals from the microbiota, and upregulate their expression of IL-17A locally (45, 46). Regional signals may induce DDX5/Rmrp association with RORγt, resulting in the transcriptional activation of multiple loci that enable T_{H}17 cell effector functions. The present inventors' finding that DDX5 was required for the differentiation of ‘pathogenic’ T_{H}17 cells (2, 29, 30) suggests that strategies to interfere with this function may be of therapeutic benefit. A better understanding of this transcriptional regulatory system may provide new approaches for therapeutic intervention in autoimmune diseases and immune deficiencies in CHH patients.

### GENERAL REFERENCES

1. Weaver, C. T., Hatton, R. D., Mangan, P. R. & Harrington, L. E. IL-17 family cytokines and the expanding diversity of effector T cell lineages. Annu. Rev. Immunol. 25, 821-852 (2007).
2. Ivanov, I. I. et al. The orphan nuclear receptor RORγt directs the differentiation program of proinflammatory IL-17+ T helper cells. Cell 126, 1121-1133 (2006).
3. Leppkes, M. et al. RORγ-expressing T_{H}17 cells induce murine chronic intestinal inflammation via redundant effects of IL-17A and IL-17F. Gastroenterology 136, 257-267 (2009).
4. Genovese, M. C. et al. LY2439821, a humanized anti-interleukin-17 monoclonal antibody, in the treatment of patients with rheumatoid arthritis: a phase I randomized, double-blind, placebo-controlled, proof-of-concept study. Arthritis Rheum. 62, 929-939 (2010).
5. Huh, J. R. et al. Digoxin and its derivatives suppress T_{H}17 cell differentiation by antagonizing RORγt activity. Nature 472, 486-490 (2011).
6. Kondo, Y. et al. Involvement of RORγt-overexpressing T cells in the development of autoimmune arthritis in mice. Arthritis Res. Ther. 17, 105 (2015).
7. Gaffen, S. L., Jain, R., Garg, A. V. & Cua, D. J. The IL-23-IL-17 immune axis: from mechanisms to therapeutic testing. Nature Rev. Immunol. 14, 585-600 (2014).
8. Ciofani, M. et al. A validated regulatory network for T_{H}17 cell specification. Cell 151, 289-303 (2012).
9. O'Malley, B. W. & Kumar, R. Nuclear receptor coregulators in cancer biology. Cancer Res. 69, 8217-8222 (2009).
10. Huang, Y. & Liu, Z. R. The ATPase, RNA unwinding, and RNA binding activities of recombinant p68 RNA helicase. J. Biol. Chem. 277, 12810-12815 (2002).
11. Fuller-Pace, F. V. & Moore, H. C. RNA helicases p68 and p72: multifunctional proteins with important implications for cancer development. Future Oncol. 7,239-251 (2011).
12. Clark, E. L. et al. The RNA helicase p68 is a novel androgen receptor coactivator involved in splicing and is overexpressed in prostate cancer. Cancer Res. 68, 7938-7946 (2008).
13. Linder, P. & Jankowsky, E. From unwinding to clamping—the DEAD box RNA helicase family. Nature Rev. Mol. Cell Biol. 12, 505-516 (2011).
14. Arun, G., Akhade, V. S., Donakonda, S. & Rao, M. R. mrhl RNA, a long noncoding RNA, negatively regulates Wnt signaling through its protein partner DdxS/p68 in mouse spermatogonial cells. Mol. Cell. Biol. 32, 3140-3152 (2012).
15. Caretti, G. et al. The RNA helicases p68/p72 and the noncoding RNA SRA are coregulators of MyoD and skeletal muscle differentiation. Dev. Cell 11, 547-560 (2006).
16. Lin, C., Yang, L., Yang, J. J., Huang, Y. & Liu, Z. R. ATPase/helicase activities of p68 RNA helicase are required for pre-mRNA splicing but not for assembly of the spliceosome. Mol. Cell. Biol. 25, 7484-7493 (2005). 17. Jalal, C., Uhlmann-Schiffler, H. & Stahl, H. Redundant role of DEAD box proteins p68 (Ddx5) and p72/p82 (Ddx17) in ribosome biogenesis and cell proliferation. Nucleic Acids Res. 35, 3590-3601 (2007). 18. Rosenbluh, J. et al. RMRP is a non-coding RNA essential for early murine development. PLoS ONE 6, e26270 (2011).
19. Hsieh, C. L. et al. The gene for the RNA component of the mitochondrial RNA-processing endoribonuclease is located on human chromosome 9p and on mouse chromosome 4. Genomics 6, 540-544 (1990).
20. Esakova, O. & Krasilnikov, A. S. Of proteins and RNA: the RNase P/MRP family. RNA 16, 1725-1747 (2010).
21. Makitie, O., Kaitila, I. & Savilahti, E. Susceptibility to infections and in vitro immune functions in cartilage-hair hypoplasia. Eur. J. Pediatr. 157, 816-820 (1998).
22. Bonafé, L. et al. Evolutionary comparison provides evidence for pathogenicity of RMRP mutations. PLoS Genet. 1, e47 (2005).
23. Bacchetta, J. et al. Autoimmune hypoparathyroidism in a 12-year-old girl with McKusick cartilage hair hypoplasia. Pediatr. Nephroi. 24, 2449-2453 (2009).
24. Jensen, E. D. et al. p68 (Ddx5) interacts with Runx2 and regulates osteoblast differentiation. J. Cell. Biochem. 103, 1438-1451 (2008).
25. Dardenne, E. et al. RNA helicases DDX5 and DDX17 dynamically orchestrate transcription, miRNA, and splicing programs in cell differentiation. Cell Rep. 7,1900-1913 (2014).
26. Wortham, N. C. et al. The DEAD-box protein p72 regulates ERα-/oestrogen-dependent transcription and cell growth, and is associated with improved survival in ERα-positive breast cancer. Oncogene 28, 4053-4064 (2009).
27. Ivanov, I. I. et al. Induction of intestinal T_{H}17 cells by segmented filamentous bacteria. Cell 139, 485-498 (2009).
28. Powrie, F. et al. Inhibition of Th1 responses prevents inflammatory bowel disease in scid mice reconstituted with CD45RBhi CD4+ T cells. Immunity 1, 553-562 (1994).
29. Hirota, K. et al. Fate mapping of IL-17-producing T cells in inflammatory responses. Nature Immunol. 12, 255-263 (2011).
30. Wang, Y. et al. The transcription factors T-bet and Runx are required for the ontogeny of pathogenic interferon-y-producing T helper 17 cells. Immunity 40, 355-366 (2014).
31. Sun, Z. et al. Requirement for RORy in thymocyte survival and lymphoid organ development. Science 288, 2369-2373 (2000).
32. Chu, C., Quinn, J. & Chang, H. Y. Chromatin isolation by RNA purification (ChIRP). J. Vis. Exp. (2012).
33. Bonasio, R. & Shiekhattar, R. Regulation of transcription by long noncoding RNAs. Annu. Rev. Genet. 48, 433-455 (2014).
34. Gomez, J. A. et al. The NeST long ncRNA controls microbial susceptibility and epigenetic activation of the interferon-y locus. Cell 152, 743-754 (2013).
35. Carpenter, S. et al. A long noncoding RNA mediates both activation and repression of immune response genes. Science 341, 789-792 (2013).
36. Maenner, S., Muller, M., Frohlich, J., Langer, D. & Becker, P. B. ATP-dependent roX RNA remodeling by the helicase maleless enables specific association of MSL proteins. Mol. Cell 51, 174-184 (2013).
37. Ilik, I. A. et al. Tandem stem-loops in roX RNAs act together to mediate X chromosome dosage compensation in Drosophila. Mol. Cell 51, 156-173 (2013).
38. Calo, E. et al. RNA helicase DDX21 coordinates transcription and ribosomal RNA processing. Nature 518, 249-253 (2015).
39. Yang, J Sundrud, M. S., Skepner, J. & Yamagata, T. Targeting T_{H}17 cells in autoimmune diseases. Trends Pharmacol. Sci. 35, 493-500 (2014).
40. Lee, Y. J., Holzapfel, K. L., Zhu, J., Jameson, S. C. & Hogquist, K. A. Steady-state production of IL-4 modulates immunity in mouse strains and is determined by lineage diversity of iNKT cells. Nature Immunol. 14, 1146-1154 (2013).
41. Takatori, H. et al. Lymphoid tissue inducer-like cells are an innate source of IL-17 and IL-22. J. Exp. Med. 206, 35-41 (2009).
42. Luci, C. et al. Influence of the transcription factor RORγt on the development of NKp46+ cell populations in gut and skin. Nature Immunol. 10, 75-82 (2009).
43. Chien, Y. H., Zeng, X. & Prinz, I. The natural and the inducible: interleukin (IL)-17-producing γδ T cells. Trends Immunol. 34, 151-154 (2013).
44. Kang, H. S. et al. Gene expression profiling reveals a regulatory role for RORα and RORγ in phase I and phase II metabolism. Physiol. Genomics 31, 281-294 (2007).
45. Yang, Y. et al. Focused specificity of intestinal T_{H}17 cells towards commensal bacterial antigens. Nature 510, 152-156 (2014).
46. Sano, T. et al. An IL-23R/IL-22 circuit regulates epithelial serum amyloid A to promote local effector T_{H}17 responses. Cell (2015).

While certain of the particular embodiments of the present invention have been described and specifically exemplified above, it is not intended that the invention be limited to such embodiments. Various modifications may be made thereto without departing from the scope and spirit of the present invention, as set forth in the following claims.

### Supplementary Information

TABLE 1
DNA oligos and Antibodies

| Oliqo Name | Oliqo sequence | SEQ ID NO | Application | Ref |
|---|---|---|---|---|
| Control ASO | TAGTGCGGACCTACCCACGA | 14 | knockdown | |
| mRmrp ASO1 | GATGACGCCCCGCTGCACGC | 15 | knockdown | |
| mRmrp ASO2 | AACATGTCCCTCGTATGTAG | 16 | knockdown | |
| mRmrp ASO3 | GCGAAAGGATAAGGAACATG | 17 | knockdown | |
| mRmrp ASO4 | CTTGGCGGGCTAACAGTGAC | 18 | knockdown | |
| mRmrp ASO5 | CCCCTAGGCGAAAGGATAAG | 19 | knockdown | |
| hRmrp ASO | CGCTGAGAATGAGCCCCGTG | 20 | knockdown | |
| Universal 16S F | ACTCCTACGGGAGGCAGCAGT | 21 | QPCR | Ref #51 |
| Universal 16S R | ATTACCGCGGCTGCTGGC | 22 | QPCR | |
| SFB 16S F | GACGCTGAGGCATGAGAGCAT | 23 | QPCR | Ref #51 |
| SFB 16S R | GACGGCACGGATTGTTATTC | 24 | QPCR | |
| Reg#7: | CCTGGGTATGTCAAACAGCAGTAG | 25 | QPCR | Ref #8 |
| IL_17F_Prom_ChIP_F | | | | |
| Reg#7 | GAGTAGAAACCCTGCATTCGTCAG | 26 | QPCR | |
| IL_17F_Prom_ChIP_R | | | | |
| Reg#2: | GCAGCAGCTTCAGATATGTCC | 27 | QPCR | Ref #8 |
| IL_17A_Prom_ChIP_F | | | | |
| Reg#2: | AGATGGGAAGGGCAGAAGTT | 28 | QPCR | |
| IL_17A_Prom_ChIP_R | | | | |
| ActB_Prom_ChIP_F | TTTCAAAAGGAGGGGAGAGG | 29 | QPCR | |
| ActB_Prom_ChIP_R | TCGAGCCATAAAAGGCAACT | 30 | QPCR | |
| Reg#1 F | TTGCATGCGCCTCCTAACACATAG | 31 | QPCR | |
| Reg#1 R | TTTCTAGGTGGGTTCCTCACTGGT | 32 | QPCR | |
| Reg#3 F | AGCAAGGAATGTGGATTCAGAGGC | 33 | QPCR | |
| Reg#3 R | ACAAACACGAAGCAGTTTGGGACC | 34 | QPCR | |
| Reg#4 F | TCCAAGGGTGGCTGTTTATC | 35 | QPCR | |
| Reg#4 R | CACTTCCCCGACCTCACTAA | 36 | QPCR | |
| Reg#5 F | TCGCCTCTTGACAAACAGTG | 37 | QPCR | |
| Reg#5 R | CACGGATTTGAGCTTCTCGT | 38 | QPCR | |
| Reg#6 F | GTGAACGCAAGGGGTACAGT | 39 | QPCR | |
| Reg#6 R | CCTITGACCTTTCCCACAAA | 40 | QPCR | |
| mGAPDH F | AATGTGTCCGTCGTGGATCT | 41 | QPCR | |
| mGAPDH R | CATCGAAGGTGGAAGAGTGG | 42 | QPCR | |
| mIL17a F | TTTAACTCCCTTGGCGCAAAA | 43 | QPCR | |
| mIL17a R | CTTTCCCTCCGCATTGACAC | 44 | QPCR | |
| mIl17 F | TCCCCTGGAGGATAACACTG | 45 | OPCR | |
| mIll7 R | GGGGTCTCGAGTGATGTTGT | 46 | QPCR | |
| mRmrpQPCRfwd | CCGCAAGTCACTGTTAGCC | 47 | QPCR | |
| mRmrpQPCRrev | CACTGCCTGCGTCACTATGT | 48 | QPCR | |
| m7SK snRNA F | CCCTGCTAGAACCTCCAAAC | 49 | QPCR | |
| m7SK snRNA R | TGGAGTCTTGGAAGCTTGACT | 50 | ChIRP-QPCR | |
| m18S rRNA F | CCTGCGGCTTAATTTACTC | 51 | ChIRP-QPCR | |
| m18S rRNA R | ATGCCAGAGTCTCGTTCGTT | 52 | ChIRP-QPCR | |
| mU1 snRNA F | TGATCACGAAGGTGGTTTTCC | 53 | ChIRP-QPCR | |
| mU1 snRNA R | GCACATCCGGAGTGCAATG | 54 | ChIRP-QPCR | |
| mMalat1 F | GATAGCCCAGGAAAGAGTGC | 55 | ChIRP-QPCR | |
| mMalat1 R | CCAGCTAGCTTCATCACCAA | 56 | ChIRP-QPCR | |
| mRMRP_1_F | GCTCTGAAGGCCTGTTTCC | 57 | ChIRP-QPCR | |
| mRMRP_1_R | CCCCTAGGCGAAAGGATAAG | 58 | ChIRP-QPCR | |
| sgRNA_Rmrp_Locus | CACGGGGCTCATTCTCAGCG | 59 | CRISPR | |
| HDRdonor_Rmrp | GCTGAGCGGCGTGCAGCGGGGCGTCATC | | CRISPR | |
| | CGTCAGCTCACATAGTGACGCAGGCAGTG | | | |
| | CGACCTGGCTCGCACCAACCACACGGGG | | | |
| | CTCATTCTCAGCGCTGCTACACTTTTATTT | | | |
| | ATTTATTTTTGTTTGTTTGTATTTTGGTTTT | | | |
| | TTGACAGGGTTTCTCTGTGTAGCCCTGGC | | | |
| | TGTCCTGGCACTCACTTTGTAGACC | | | |
| | (SEQ ID NO: 60) | | | |

| | Clone/catalog | | |
|---|---|---|---|
| Antibody Name | number | Company | Application |
| Anti-Mouse CD3e | 145-2C11 | eBioscience | flow |
| Anti-Mouse CD4 | RM4-5 | eBloscience | flow |
| Anti-Mouse CD8 | 53-6.7 | eBioscience | |
| Anti-Mouse CD16/32 Fc Block | 2.4G2 | Tonbo | flow |
| Anti-Mouse CD19 | 1D3 | Tonbo | flow |
| Anti-Mouse CD25 | PC61.5 | eBioscience | flow |
| Anti-Mouse CD44 | 1M7 | eBioscience | flow |
| Anti-Mouse TCRb | H57-597 | eBioscience | flow |
| Anti-Mouse gd TCR | eBioGL3 | eBioscience | flow |
| Anti-Mouse RORgt | B2D | eBioscience | flow |
| Anti-Mouse/Rat Foxp3 | FJK-16s | eBiosdence | flow |
| Anti-Mouse B220 | RA3-6B2 | eBiosdence | flow |
| Anti-Mouse IL-17A | eBio17B7 | eBioscience | flow |
| Anti-Mouse IL-17F | 9D3.1C8 | BioLegend | flow |
| Anti-Mouse IL-22 | IL22JOP | eBioscience | flow |
| Anti-Mouse CD196 (CCR6) | 140706 | BD | flow |
| Anti-Human IL-17A | eBio64DEC17 | eBioscience | |
| Anti-Human CD4 | OKT4 | eBiosdence | |
| Anti-Human/Mouse RORyt | AFKJS-9 | eBiosdence | |
| Rabbit anti-bTubulin | 2146S | Cell Signaling | Immunoblot |
| Rabbit anti-GST | 2625S | Cell Signaling | Immunoblot |
| Mouse anti-His | sc-8036 | Santa Cruz Bio | Immunoblot |
| Mouse anti-RNA Pol II | 2629S | Cell Signaling | ChIP |
| Goat anti-DDX5 | ab10261 | Abcam | RIPseq/IP/ChIP/ |
| | | | Immunoblot/ |
| | | | microscopy |
| Rabbit anti-DDX5 | sc-26045 | Santa Cruz Bio | Immunoblot |
| Rat anti-RORgt | 14-6988-82 | eBioscience | RIPseq/IP/ChIP/ |
| | | | Immunoblot/ |
| | | | microscopy |
| Rabbit anti-GFP | A-11122 | Life Technology | RIPseq |
| Anti-RORgt (APC conjugated) | AFKJ3-9 | eBioscience | microscopy |
| Donkey anti-Goat Alexa | A-11055 | Molecular Probe | microscopy |
| Fluor488 | | | |

What is claimed is:

1. A method for screening to identify a modulator of RNA helicase DEAD-box protein 5 (DDX5) activity, the method comprising:
(a) providing a polypeptide mixture comprising RORγt polypeptide or a fragment thereof, DDX5 polypeptide, ribonucleic acid (RNA) component of Mitochondria RNA-processing endoribonuclease (Rmrp) and at least one candidate modulator agent; and
(b) detecting RORγt or RORγt fragment/DDX5/Rmrp complex formation in the presence of the candidate modulator agent and comparing that to RORγt or RORγt fragment/DDX5/Rmrp complex formation in the absence of the candidate modulator agent, wherein a change in RORγt or RORγt fragment/DDX5/Rmrp complex formation in the presence of the candidate modulator agent relative to that detected in the absence of the candidate modulator agent indicates that the at least one candidate modulator agent is a modulator of DDX5 polypeptide activity.

2. A method for screening to identify a modulator of DDX5 polypeptide activity, the method comprising:
(a) providing a polypeptide mixture comprising DDX5 polypeptide, Rmrp, and at least one candidate modulator agent; and
(b) detecting DDX5/Rmrp complex formation in the presence of the candidate modulator agent and comparing that to DDX5/Rmrp complex formation in the absence of the candidate modulator agent, wherein a change in DDX5/Rmrp complex formation in the presence of the candidate modulator agent relative to that detected in the absence of the candidate modulator agent indicates that the at least one candidate modulator agent is a modulator of DDX5 polypeptide activity.

3. A method for screening to identify an enhancer of DDX5 polypeptide activity, the method comprising:
(a) providing a polypeptide mixture comprising RORγt polypeptide or a fragment thereof, DDX5 polypeptide, and at least one candidate modulator agent; and
(b) detecting RORγt or RORγt fragment/DDX5 complex formation in the presence of the candidate modulator agent and comparing that to RORγt or RORγt fragment/DDX5 complex formation in the absence of the candidate modulator agent, wherein formation of RORγt or RORγt fragment/DDX5 complexes in the presence of the candidate modulator agent relative to that detected in the absence of the candidate modulator agent indicates that the at least one candidate modulator agent is an enhancer of DDX5 polypeptide activity.

4. The method of claim 1, wherein the change detected in the presence of the candidate modulator agent is a decrease in RORγt or RORγt fragment/DDX5/Rmrp complex formation or DDX5/Rmrp complex formation, thereby identifying the candidate modulator agent as an inhibitor of DDX5 polypeptide activity.

5. The method of claim 1, wherein the change detected in the presence of the candidate modulator agent is an increase in RORγt or RORγt fragment/DDX5/Rmrp complex formation or DDX5/Rmrp complex formation, thereby identifying the candidate modulator agent as an enhancer of DDX5 polypeptide activity.

6. The method of claim 1, wherein the screening is performed in a cell-based assay or in vitro assay comprising purified components.

7. The method of claim 6, wherein the cell-based assay comprises naive CD4+ T cells polarized under T_{H}17 inducing conditions or a cell engineered to express an exogenous gene operably linked to a RORγt-dependent responsive regulatory element.

8. The method of claim 6, wherein formation of RORγt or RORγt fragment/DDX5/Rmrp complexes, DDX5/Rmrp complexes, or RORγt or RORγt fragment/DDX5 complexes detected in the presence or absence of the candidate modulator agent is measured by a change in expression of at least one transcriptional readout in the cell-based assay or the in vitro assay comprising purified components.

9-13. (canceled)

14. The method of claim 1, wherein the RORγt fragment comprises the ligand binding domain of the RORγt polypeptide.

15. The method of claim 1, wherein the candidate modulator agent is a small organic molecule, a peptide, or a nucleic acid.

16. The method of claim 15, wherein the nucleic acid is a single-strand or double-strand DNA or RNA.

17. The method of claim 16, wherein the single-strand DNA is an anti-sense oligonucleotide.

18. The method of claim 17, wherein the anti-sense oligonucleotide is specific for Rmrp or DDX5.

19. A method for treating a mammalian subject afflicted with an inflammatory condition or autoimmune disease associated with T_{H}17 cell mediated pathology, the method comprising administering the inhibitor of DDX5 polypeptide activity of claim 4 to the subject, wherein the inhibitor of DDX5 polypeptide activity reduces T_{H}17 cell activity in the subject and thereby treats the mammalian subject.

20. The method of claim 19, wherein the inflammatory condition or autoimmune disease is Crohn's disease, ulcerative colitis, multiple sclerosis, rheumatoid arthritis, or psoriasis.

21. The method of claim 19, wherein the mammalian subject is a human.

22. A method for treating a mammalian subject afflicted with a cancer, the method comprising administering the enhancer of DDX5 polypeptide activity of claim 3 to the subject, wherein the enhancer of DDX5 polypeptide activity increases T_{H}17 cell activity in the mammalian subject and thereby treats the subject.

23. The method of claim 22, wherein the cancer is gastric adenocarcinoma, lung cancer, ovarian cancer, melanoma, glioblastoma, or pancreatic cancer.

24. The method of claim 22, wherein the mammalian subject is a human.

25-32. (canceled)


SEQUENCE LISTING (65 sequences)

SEQ ID NO: 1 - protein, 518 aa, Homo sapiens
MDRAPQRQHRASRELLAAKKTHTSQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 2 - protein, 497 aa, Homo sapiens
MRTQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 3 - protein, 495 aa, Mus musculus
MRTQIEVIPCKICGDKSSGIHYGVITCEGCKGFFRRSQQCNVAYSCTRQQNCPIDRTSRNRCQHCRLQKCLALGMSRDAVKFGRMSKKQRDSLHAEVQKQLQQQQQQEQVAKTPPAGSRGADTLTYTLGLSDGQLPLGASPDLPEASACPPGLLRASGSGPPYSNTLAKTEVQGASCHLEYSPERGKAEGRDSIYSTDGQLTLGRCGLRFEETRHPELGEPEQGPDSHCIPSFCSAPEVPYASLTDIEYLVQNVCKSFRETCQLRLEDLLRQRTNLFSREEVTSYQRKSMWEMWERCAHHLTEAIQYVVEFAKRLSGFMELCQNDQIILLKAGAMEVVLVRMCRAYNANNHTVFFEGKYGGVELFRALGCSELISSIFDFSHFLSALCFSEDEIALYTALVLINANRPGLQEKRRVEHLQYNLELAFHHHLCKTHRQGLLAKLPPKGKLRSLCSQHVEKLQIFQHLHPIVVQAAFPPLYKELFSTDVESPEGLSK

SEQ ID NO: 4 - protein, 614 aa, Homo sapiens
MSGYSSDRDRGRDRGFGAPRFGGSRAGPLSGKKFGNPGEKLVKKKWNLDELPKFEKNFYQEHPDLARRTAQEVETYRRSKEITVRGHNCPKPVLNFYEANFPANVMDVIARQNFTEPTAIQAQGWPVALSGLDMVGVAQTGSGKTLSYLLPAIVHINHQPFLERGDGPICLVLAPTRELAQQVQQVAAEYCRACRLKSTCIYGGAPKGPQIRDLERGVEICIATPGRLIDFLECGKTNLRRTTYLVLDEADRMLDMGFEPQIRKIVDQIRPDRQTLMWSATWPKEVRQLAEDFLKDYIHINIGALELSANHNILQIVDVCHDVEKDEKLIRLMEEIMSEKENKTIVFVETKRRCDELTRKMRRDGWPAMGIHGDKSQQERDWVLNEFKHGKAPILIATDVASRGLDVEDVKFVINYDYPNSSEDYIHRIGRTARSTKTGTAYTFFTPNNIKQVSDLISVLREANQAINPKLLQLVEDRGSGRSRGRGGMKDDRRDRYSAGKRGGFNTFRDRENYDRGYSSLLKRDFGAKTQNGVYSAANYTNGSFGSNFVSAGIQTSFRTGNPTGTYQNGYDSTQQYGSNVPNMHNGMNQQAYAYPATAAAPMIGYPMPTGYSQ

SEQ ID NO: 5 - protein, 615 aa, Mus musculus
MSSYSSDRDRGRDRGFGAPRFGGSRTGPLSGKKFGNPGEKLVKKKWNLDELPKFEKNFYQEHPDLARRTAQEVDTYRRSKEITVRGHNCPKPVLNFYEANFPANVMDVIARQNFTEPTAIQAQGWPVALSGLDMVGVAQTGSGKTLSYLLPAIVHINHQPFLERGDGPICLVLAPTRELAQQVQQVAAEYCRACRLKSTCIYGGAPKGPQIRDLERGVEICIATPGRLIDFLECGKTNLRRTTYLVLDEADRMLDMGFEPQIRKIVDQIRPDRQTLMWSATWPKEVRQLAEDFLKDYIHINIGALELSANHNILQIVDVCHDVEKDEKLIRLMEEIMSEKENKTIVFVETKRRCDELTRKMRRDGWPAMGIHGDKSQQERDWVLNEFKHGKAPILIATDVASRGLDVEDVKFVINYDYPNSSEDYIHRIGRTARSTKTGTAYTFFTPNNIKQVSDLISVLREANQAINPKLLQLVEDRGSGRSRGRGGMKDDRRDRYSAGKRGGFNTFRDRENYDRGYSNLLKRDFGAKTQNGVYSAANYTNGSFGSNFVSAGIQTSFRTGNPTGTYQNGYDSTQQYGSNVANMHNGMNQQAYAYPATAAAAPMIGYPMPTGYSQ

SEQ ID NO: 6 - DNA, 277 bp, Homo sapiens
ggttcgtgctgaaggcctgtatcctaggctacacactgaggactctgttcctcccctttccgcctaggggaaagtccccggacctcgggcagagagtgccacgtgcatacgcacgtagacattccccgcttcccactccaaagtccgccaagaagcgtatcccgctgagcggcgtggcgcgggggcgtcatccgtcagctccctctagttacgcaggcagtgcgtgtccgcgcaccaaccacacggggctcattctcagcgcggctgtaaaaaaaaa

SEQ ID NO: 7 - DNA, 275 bp, Mus musculus
agctcgctctgaaggcctgtttcctaggctacatacgagggacatgttccttatcctttcgcctaggggaaagtccccggaccacgggcagagagtgccgcgtgcacacgcgcgtagacttcccccgcaagtcactgttagcccgccaagaagcgacccctccggggcgagctgagcggcgtgcagcggggcgtcatccgtcagctcacatagtgacgcaggcagtgcgacctggctcgcaccaaccacacggggctcattctcagcgcggctac

SEQ ID NO: 8 - DNA, 20 bp, Artificial Sequence
  Synthetic oligonucleotide
taggaaacaggccttcagag

SEQ ID NO: 9 - DNA, 20 bp, Artificial Sequence
  Synthetic oligonucleotide
aacatgtccctcgtatgtag

SEQ ID NO: 10 - DNA, 20 bp, Artificial Sequence
  Synthetic oligonucleotide
cccctaggcgaaaggataag

SEQ ID NO: 11 - DNA, 20 bp, Artificial Sequence
  Synthetic oligonucleotide
aacagtgacttgcgggggaa

SEQ ID NO: 12 - DNA, 20 bp, Artificial Sequence
  Synthetic oligonucleotide
ctatgtgagctgacggatga

SEQ ID NO: 13 - protein, 13 aa, Homo sapiens
YGSPSFRSTPEAP

SEQ ID NO: 14 - DNA, 20 bp, Artificial Sequence
  Synthetic oligonucleotide
tagtgcggacctacccacga

SEQ ID NO: 15 - DNA, 20 bp, Artificial Sequence
  Synthetic oligonucleotide
gatgacgccccgctgcacgc

SEQ ID NO: 16 - DNA, 20 bp, Artificial Sequence
  Synthetic oligonucleotide
aacatgtccctcgtatgtag

SEQ ID NO: 17 - DNA, 19 bp, Artificial Sequence
  Synthetic oligonucleotide
gcgaaaggataggaacatg

SEQ ID NO: 18 - DNA, 20 bp, Artificial Sequence
  Synthetic oligonucleotide
cttggcgggctaacagtgac

SEQ ID NO: 19 - DNA, 20 bp, Artificial Sequence
  Synthetic oligonucleotide
cccctaggcgaaaggataag

SEQ ID NO: 20 - DNA, 20 bp, Artificial Sequence
  Synthetic oligonucleotide
cgctgagaatgagccccgtg

SEQ ID NO: 21 - DNA, 21 bp, Artificial Sequence
  Synthetic oligonucleotide
actcctacgggaggcagcagt

SEQ ID NO: 22 - DNA, 18 bp, Artificial Sequence
  Synthetic oligonucleotide
attaccgcggctgctggc

SEQ ID NO: 23 - DNA, 21 bp, Artificial Sequence
  Synthetic oligonucleotide
gacgctgaggcatgagagcat

SEQ ID NO: 24 - DNA, 20 bp, Artificial Sequence
  Synthetic oligonucleotide
gacggcacggattgttattc

SEQ ID NO: 25 - DNA, 24 bp, Artificial Sequence
  Synthetic oligonucleotide
cctgggtatgtcaaacagcagtag

SEQ ID NO: 26 - DNA, 24 bp, Artificial Sequence
  Synthetic oligonucleotide
gagtagaaaccctgcattcgtcag

SEQ ID NO: 27 - DNA, 21 bp, Artificial Sequence
  Synthetic oligonucleotide
gcagcagcttcagatatgtcc

SEQ ID NO: 28 - DNA, 20 bp, Artificial Sequence
  Synthetic oligonucleotide
agatgggaagggcagaagtt

SEQ ID NO: 29 - DNA, 20 bp, Artificial Sequence
  Synthetic oligonucleotide
tttcaaaaggaggggagagg

SEQ ID NO: 30 - DNA, 20 bp, Artificial Sequence
  Synthetic oligonucleotide
tcgagccataaaaggcaact

SEQ ID NO: 31 - DNA, 24 bp, Artificial Sequence
  Synthetic oligonucleotide
ttgcatgcgcctcctaacacatag

SEQ ID NO: 32 - DNA, 24 bp, Artificial Sequence
  Synthetic oligonucleotide
tttctaggtgggttcctcactggt

SEQ ID NO: 33 - DNA, 24 bp, Artificial Sequence
  Synthetic oligonucleotide
agcaaggaatgtggattcagaggc

SEQ ID NO: 34 - DNA, 24 bp, Artificial Sequence
  Synthetic oligonucleotide
acaaacacgaagcagtttgggacc

SEQ ID NO: 35 - DNA, 20 bp, Artificial Sequence
  Synthetic oligonucleotide
tccaagggtggctgtttatc

SEQ ID NO: 36 - DNA, 20 bp, Artificial Sequence
  Synthetic oligonucleotide
cacttccccgacctcactaa

SEQ ID NO: 37 - DNA, 20 bp, Artificial Sequence
  Synthetic oligonucleotide
tcgcctcttgacaaacagtg

SEQ ID NO: 38 - DNA, 20 bp, Artificial Sequence
  Synthetic oligonucleotide
cacggatttgagcttctcgt

SEQ ID NO: 39 - DNA, 20 bp, Artificial Sequence
  Synthetic oligonucleotide
gtgaacgcaaggggtacagt

SEQ ID NO: 40 - DNA, 20 bp, Artificial Sequence
  Synthetic oligonucleotide
cctttgacctttcccacaaa

SEQ ID NO: 41 - DNA, 20 bp, Artificial Sequence
  Synthetic oligonucleotide
aatgtgtccgtcgtggatct

SEQ ID NO: 42 - DNA, 20 bp, Artificial Sequence
  Synthetic oligonucleotide
catcgaaggtggaagagtgg

SEQ ID NO: 43 - DNA, 21 bp, Artificial Sequence
  Synthetic oligonucleotide
tttaactcccttggcgcaaaa

SEQ ID NO: 44 - DNA, 20 bp, Artificial Sequence
  Synthetic oligonucleotide
ctttccctccgcattgacac

SEQ ID NO: 45 - DNA, 20 bp, Artificial Sequence
  Synthetic oligonucleotide
tcccctggaggataacactg

SEQ ID NO: 46 - DNA, 20 bp, Artificial Sequence
  Synthetic oligonucleotide
ggggtctcgagtgatgttgt

SEQ ID NO: 47 - DNA, 19 bp, Artificial Sequence
  Synthetic oligonucleotide
ccgcaagtcactgttagcc

SEQ ID NO: 48 - DNA, 20 bp, Artificial Sequence
  Synthetic oligonucleotide
cactgcctgcgtcactatgt

SEQ ID NO: 49 - DNA, 20 bp, Artificial Sequence
  Synthetic oligonucleotide
ccctgctagaacctccaaac

SEQ ID NO: 50 - DNA, 21 bp, Artificial Sequence
  Synthetic oligonucleotide
tggagtcttggaagcttgact

SEQ ID NO: 51 - DNA, 20 bp, Artificial Sequence
  Synthetic oligonucleotide
cctgcggcttaatttgactc

SEQ ID NO: 52 - DNA, 20 bp, Artificial Sequence
  Synthetic oligonucleotide
atgccagagtctcgttcgtt

SEQ ID NO: 53 - DNA, 21 bp, Artificial Sequence
  Synthetic oligonucleotide
tgatcacgaaggtggttttcc

SEQ ID NO: 54 - DNA, 19 bp, Artificial Sequence
  Synthetic oligonucleotide
gcacatccggagtgcaatg

SEQ ID NO: 55 - DNA, 20 bp, Artificial Sequence
  Synthetic oligonucleotide
gatagcccaggaaagagtgc

SEQ ID NO: 56 - DNA, 20 bp, Artificial Sequence
  Synthetic oligonucleotide
ccagctagcttcatcaccaa

SEQ ID NO: 57 - DNA, 19 bp, Artificial Sequence
  Synthetic oligonucleotide
gctctgaaggcctgtttcc

SEQ ID NO: 58 - DNA, 20 bp, Artificial Sequence
  Synthetic oligonucleotide
cccctaggcgaaaggataag

SEQ ID NO: 59 - DNA, 20 bp, Artificial Sequence
  Synthetic oligonucleotide
cacggggctcattctcagcg

SEQ ID NO: 60 - DNA, 199 bp, Artificial Sequence
  Synthetic oligonucleotide
gctgagcggcgtgcagcggggcgtcatccgtcagctcacatagtgacgcaggcagtgcgacctggctcgcaccaaccacacggggctcattctcagcgctgctacacttttatttatttattttgtttgtttgtattttggttttttgacagggtttctctgtgtagccctggctgtcctggcactcactttgtagacc

SEQ ID NO: 61 - DNA, 3084 bp, Homo sapiens
gccaggtgctcccgccttccaccctccgccctcctccctcccctgggccctgctccctgccctcctgggcagccagggcagccaggacggcaccaagggagctgccccatggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtgacctggaagagggactccttgcctctccctatggcctgctggcccacctccctggaccccgttccaccctcacccttttcctttcccatgaaccctggagggtggtccccaccagctctttggaagtgagcagatgctgcggctggctttctgtcagcaggccggcctggcagtgggacaatcgccagagggtggggctggcagaacaccatctccagcctcagctttgacctgtctcatttcccatattccttcacacccagcttctggaaggcatggggtggctgggatttaaggacttctgggggaccaagacatcctcaagaaaacaggggcatccagggctccctggatgaatagaatgcaattcattcagaagctcagaagctaagaataagcctttgaaatacctcattgcatttccctttgggcttcggcttggggagatggatcaagctcagagactggcagtgagagcccagaaggacctgtataaaatgaatctggagctttacattttctgcctctgccttcctcccagctcagcaaggaagtatttgggcaccctaccctttacctggggtctaaccaaaaatggatgggatgaggatgagaggctggagataattgttttatgggatttgggtgtgggactagggtacaatgaaggccaagagcatctcagacatagagttaaaactcaaacctcttatgtgcactttaaagatagactttaggggctggcacaaatctgatcagagacacatatccatacacaggtgaaacacatacagactcaacagcaatcatgcagttccagagacacatgaacctgacacaatctctcttatccttgaggccacagcttggaggagcctagaggcctcaggggaaagtcccaatcctgagggaccctcccaaacatttccatggtgctccagtccactgatcttgggtctggggtgatccaaataccaccccagctccagctgtcttctaccactagaagacccaagagaagcagaagtcgctcgcactggtcagtcggaaggcaagatcagatcctggaggactttcctggcctgcccgccagccctgctcttgttgtggagaaggaagcagatgtgatcacatcaccccgtcattgggcaccgctgactccagcatggaggacaccagggagcagggcctgggcctgtttccccagctgtgatcttgcccagaacctctcttggcttcataaacagctgtgaaccctcccctgagggattaacagcaatgatgggcagtcgtggagttgggggggttgggggtgggattgtgtcctctaaggggacgggttcatctgagtaaacataaaccccaacttgtgccattctttataaaatgattttaaaggcaaaaaaaaaaaaaaaaaaaa

SEQ ID NO: 62 - DNA, 3054 bp, Homo sapiens
agagagctaggtgcagagcttcaggctgaggcgctgctgagagggcctcgccccgcctctgccgccagctgcaccccactcctggaccaccccctgctgagaaggacagggagccaaggccggcagagccaaggctcagtcatgagaacacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtgacctggaagagggactccttgcctctccctatggcctgctggcccacctccctggaccccgttccaccctcacccttttcctttcccatgaaccctggagggtggtccccaccagctctttggaagtgagcagatgctgcggctggctttctgtcagcaggccggcctggcagtgggacaatcgccagagggtggggctggcagaacaccatctccagcctcagctttgacctgtctcatttcccatattccttcacacccagcttctggaaggcatggggtggctgggatttaaggacttctgggggaccaagacatcctcaagaaaacaggggcatccagggctccctggatgaatagaatgcaattcattcagaagctcagaagctaagaataagcctttgaaatacctcattgcatttccctttgggcttcggcttggggagatggatcaagctcagagactggcagtgagagcccagaaggacctgtataaaatgaatctggagctttacattttctgcctctgccttcctcccagctcagcaaggaagtatttgggcaccctaccctttacctggggtctaaccaaaaatggatgggatgaggatgagaggctggagataattgttttatgggatttgggtgtgggactagggtacaatgaaggccaagagcatctcagacatagagttaaaactcaaacctcttatgtgcactttaaagatagactttaggggctggcacaaatctgatcagagacacatatccatacacaggtgaaacacatacagactcaacagcaatcatgcagttccagagacacatgaacctgacacaatctctcttatccttgaggccacagcttggaggagcctagaggcctcaggggaaagtcccaatcctgagggaccctcccaaacatttccatggtgctccagtccactgatcttgggtctggggtgatccaaataccaccccagctccagctgtcttctaccactagaagacccaagagaagcagaagtcgctcgcactggtcagtcggaaggcaagatcagatcctggaggactttcctggcctgcccgccagccctgctcttgttgtggagaaggaagcagatgtgatcacatcaccccgtcattgggcaccgctgactccagcatggaggacaccagggagcagggcctgggcctgtttccccagctgtgatcttgcccagaacctctcttggcttcataaacagctgtgaaccctcccctgagggattaacagcaatgatgggcagtcgtggagttgggggggttgggggtgggattgtgtcctctaaggggacgggttcatctgagtaaacataaaccccaacttgtgccattctttataaaatgattttaaaggcaaaaaaaaaaaaaaaaaaaa

SEQ ID NO: 63 - DNA, 2576 bp, Mus musculus
gtggagcagagcttaaacccccctgcccagaaacactgggggagagctttgtgcagatctaagggctgaggcacccgctgagagggcttcaccccacctccactgccagctgtgtgctgtcctgggctaccctactgaggaggacagggagccaagttctcagtcatgagaacacaaattgaagtgatcccttgcaagatctgtggggacaagtcatctgggatccactacggggttatcacctgtgaggggtgcaagggcttcttccgccgcagccagcagtgtaatgtggcctactcctgcacgcgtcagcagaactgccccattgaccgaaccagccgcaaccgatgccagcattgccgcctgcagaagtgcctggctctgggcatgtcccgagatgctgtcaagtttggccgaatgtccaagaagcagagggacagtctacatgcagaagtgcagaaacaactgcaacagcagcagcaacaggaacaagtggccaagactcctccagctgggagccgcggagcagacacacttacatacactttagggctctcagatgggcagctaccactgggcgcctcacctgacctacccgaggcctctgcttgtccccctggcctcctgagagcctcaggctctggcccaccatattccaataccttggccaaaacagaggtccagggggcctcctgccaccttgagtatagtccagaacgaggcaaagctgaaggcagagacagcatctatagcactgacggccaacttactcttggaagatgtggacttcgttttgaggaaaccaggcatcctgaacttggggaaccagaacagggtccagacagccactgcattcccagtttctgcagtgccccagaggtaccatatgcctctctgacagacatagagtacctggtacagaatgtctgcaagtccttccgagagacatgccagctgcgactggaggaccttctacggcagcgcaccaacctcttttcacgggaggaggtgaccagctaccagaggaagtcaatgtgggagatgtgggagcgctgtgcccaccacctcactgaggccattcagtatgtggtggagtttgccaagcggctttcaggcttcatggagctctgccagaatgaccagatcatactactgaaagcaggagcaatggaagtcgtcctagtcagaatgtgcagggcctacaatgccaacaaccacacagtcttttttgaaggcaaatacggtggtgtggagctgtttcgagccttgggctgcagcgagctcatcagctccatatttgacttttcccacttcctcagcgccctgtgtttttctgaggatgagattgccctctacacggccctggttctcatcaatgccaaccgtcctgggctccaagagaagaggagagtggaacatctgcaatacaatttggaactggctttccatcatcatctctgcaagactcatcgacaaggcctcctagccaagctgccacccaaaggaaaactccggagcctgtgcagccaacatgtggaaaagctgcagatcttccagcacctccaccccatcgtggtccaagccgccttccctccactctataaggaactcttcagcactgatgttgaatcccctgaggggctgtcaaagtgatctggaggaaggacaactttctatttccttcagccctctgacccgtctccctggactcccttcacccagcctttccctttctgcactctatgaagggtggtatccctaggagtaagcaaatcctaagactgattttctgcccctaggcttgccttgtaggacaacagcagcaagtgatggagaaaaggcttgttatgtttgatttcccataagttccaccctggcttctggaagctgtggggtagatgggatagagataggatgaccaagtcaaataaaaaacagactgacaatcagcagggataaatccaggtacctgggataaggagaactcaaatctaggcttgaaagctaataacagtcctttcaatacctcattgtatttccccatgggtcctcctggggggacatggatctagctcagagactggtggcaagcccccagaaggacctgtatataataagaatatagattcctgagacttttctgcctttcttcttcctagttaagaaatgttgttgaccccctctgcctgttttctgggacctaaaatgcctggatgtgtaaagaatgagggtggggtggagataaggtcccaagataactgttttatggggtttgggtatgaagaaaaacatcactggaaaaattagaatggaaacctctttgcacactttaaaagtgtcagattcgttagcagtctaatcagagacacacatccacacaggtggagcacacagaggctctgcccccagtgacaccattctgtagactttccctctggcacacaatctcttccttgaggttgcagctctgagaagcctgaggttctaattcatacaggacaccagaattcatcccagctccagctgtcctctgtccctaagagaagcagaggaccggatactaaccagccggaaaa

SEQ ID NO: 64 - DNA, 3938 bp, Homo sapiens
agaagaaggtgaggtcaagagagtgcagaatgaggcattccaatggtgggtgggccctgacctgagagagtggcgcggggaggggtgaaagcgcggcgatcctggaacgccagcgggcgttgcggcctatgcgcgaggggcggggcgattaggtcatagagcggctcccagcgttccctgcggcgtaggaggcggtccagactataaaagcggctgccggaaagcggccggcacctcattcatttctaccggtctctagtagtgcagcttcggctggtgtcatcggtgtccttcctccgctgccgcccccgcaaggcttcgccgtcatcgaggccatttccagcgacttgtcgcacgcttttctatatacttcgttccccgccaaccgcaaccattgacgccatgtcgggttattcgagtgaccgagaccgcggccgggaccgagggtttggtgcacctcgatttggaggaagtagggcagggcccttatctggaaagaagtttggaaaccctggggagaaattagttaaaaagaagtggaatcttgatgagctgcctaaatttgagaagaatttttatcaagagcaccctgatttggctaggcgcacagcacaagaggtggaaacatacagaagaagcaaggaaattacagttagaggtcacaactgcccgaagccagttctaaatttttatgaagccaatttccctgcaaatgtcatggatgttattgcaagacagaatttcactgaacccactgctattcaagctcagggatggccagttgctctaagtggattggatatggttggagtggcacagactggatctgggaaaacattgtcttatttgcttcctgccattgtccacatcaatcatcagccattcctagagagaggcgatgggcctatttgtttggtgctggcaccaactcgggaactggcccaacaggtgcagcaagtagctgctgaatattgtagagcatgtcgcttgaagtctacttgtatctacggtggtgctcctaagggaccacaaatacgtgatttggagagaggtgtggaaatctgtattgcaacacctggaagactgattgactttttagagtgtggaaaaaccaatctgagaagaacaacctaccttgtccttgatgaagcagatagaatgcttgatatgggctttgaaccccaaataaggaagattgtggatcaaataagacctgataggcaaactctaatgtggagtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatccaatgccaacaggatattcccaataagactttagaagtatatgtaaatgtctgtttttcataattgctctttatattgtgtgttatctgacaagatagttatttaagaaacatgggaattgcagaaatgactgcagtgcagcagtaattatggtgcactttttcgctatttaagttggatatttctctacattcctgaaacaatttttaggttttttttgtactagaaaatgcaggcagtgttttcacaaaagtaaatgtacagtgatttgaaatacaataaatgaaggcaatgcatggccttccaataaaaaatatttgaagactgaattaagtggaaattgtactttattttatataatgtcatgtaaaactttgcttaagatggtctggttttttttttgtttttgtttggttttttttttccatgaaaacaaatgactgttcctttttatttaatttgggaggcagggggaatcagaaggcccttctttataatgagctattcatattgcaggagtcagaatgaattgatacaggtgaatttttagttacaggctaaattgcataaaagctttgtcagcttccagcatcaggggagtcatttaatagcctttttccttatttgctagtatggttaaatgagaaaatagtaaaatagatacaaagtcatctatatagtgtgagaacgtgggtgactttttcaaagtttataatttaaaaagctccaaataactggctttttcaagagacttatactcatgctcttggctatactgtgaattactgaaatgttgaacaaacctgtgaaagacatacattagccctttaagatggccaggagctaagcttgagtctcctttactgaatttcgttcttagtgcaggttacttgtagattctagtcttcacaggctccctggggctcttaactagtcacactgggagtcatgaatgtctttccaataattcagggaattctagagatcctcaaactgtaaggtctattcatactcaacacaaggaaaaaacctcattaaaattaatgactaatcaggaggcaacgtaaccaaaagcacagtgaatgaaagttttcatggtaggttcaacatgggtttattgctagaaagatccaggggatagctttaggtttaacttcggctcaccaacgtaactttctaatcatttatttcagtaatagctagaagtgggtctgaatgttttcccagagtctgatacgtgtttttttttgccagaagagaggtcttcaggagacttcatttaaattctgattattaaactgaggctttaattgatgttaatgccttatgtcaaatgtaaagttagaatttgctagggctgggatagggagtgatatttctaggacttagacattgaaaactaattcagcctgtagtaacctggatggttttcaatggcatggttagtcaaattcatggttttaaacttagaagcagctttcgggggagagggtaggttggagcatttattacatattttactgtttaatgtcttaaccgtgggccttttaatttgtaaacactgaaatgattgttgggctgtggaaaacatttacctatttaccttggaagttttaaaagacagtccactttttagcatgtgtgttgtgtccagcctgtggtcgtcttaactaataaatgtgatttttctccccaaaaaaaaaaaaaaaaaaaaaaa

SEQ ID NO: 65 - DNA, 3520 bp, Mus musculus
acctcattcatttccatcggtccttgccctcgcagctccggccctcgacaccaccggcccctccgctggtgacgcccccgcagaggcttcgccgtcgccgaggccttctctccgcgactcgtcgccgcaggattctactttcactgggttccgcccgcacaccgccaccattgacgccatgtcgagttattctagtgaccgagaccgcggccgggatcgagggtttggtgcacctcgatttggagggagtagaacaggacccctctctggaaagaagtttggaaatcctggggagaaactagttaaaaagaagtggaatcttgatgagctgcccaaatttgagaagaatttttatcaagaacaccctgatttggcaaggcgcaccgcacaagaggtagatacatacagaagaagcaaggaaattacagttagaggtcacaactgtccaaaacctgttctgaatttttatgaagcaaactttcctgcgaatgtcatggatgtgattgcaaggcagaactttactgaacccactgctattcaagctcagggctggccagttgctctcagtggattggatatggttggagtagctcagactggatctgggaaaacattatcttatttgctgcctgccattgtacacataaaccaccagccattcctagagagaggtgatgggcctatttgcttggtgctggcaccaactcgagaactggcacagcaggtgcagcaagtggctgctgaatattgtcgagcttgtcgcttgaagtctacttgcatctatggtggtgctcccaaaggaccacagattcgtgatttggaaagaggtgtggaaatctgtattgcaacacctggaagactgattgactttttagagtgtgggaaaaccaatctgagaagaacaacttaccttgtccttgatgaagctgataggatgcttgatatgggatttgaaccccagataaggaaaattgtggatcaaataagacctgataggcaaacactaatgtggagtgcaacttggccaaaagaagtaagacagcttgctgaagatttcctgaaagactatattcatatcaatattggtgcactggaactgagtgcaaaccataacattcttcagattgtggatgtatgtcatgatgtcgaaaaggatgaaaagcttattcgtctgatggaagaaatcatgagtgagaaggagaataaaactattgtttttgttgaaaccaaaagaagatgtgatgaacttaccagaaaaatgaggagagatgggtggcctgccatgggcatccatggtgacaagagtcagcaggaacgtgactgggttctaaatgaattcaaacatggaaaagctcctattctgattgctaccgatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgggaagctaatcaagcaattaatcccaagttgcttcagttggtcgaagacagaggttcaggtcgttccaggggtagaggaggcatgaaggacgatcgtcgtgacagatactctgcaggcaaaaggggtggatttaatacctttagagacagggaaaactatgacagaggctactctaatctgcttaagagagattttggggctaaaactcagaatggtgtttacagtgctgcaaattacaccaatgggagctttggaagtaattttgtatctgctggcatacagaccagttttaggactggtaatccaacagggacttaccagaacggttatgatagcactcagcaatatggaagtaatgttgcaaatatgcacaatggtatgaaccaacaggcatatgcatatcctgctaccgcagctgctgcgcctatgattggctatcccatgccaacagggtattctcaataagactttagaagtatatgtaaatgtctgtttttcataattgctctttatattgtgtgttatcagacaagatagttatttaagaaacatgggaaatgcagaaatgactgcagtgcagcagtaattatggtgcacttttttcgctatttaagttggatatttctctacattcctgaaacaatttttaggttttttttgtactagaaaatgcaggcagtgttttcacaaaagtaactgtacagtgatttcaaatacaataaatgaaggcaatgcatggccttccaataaaagatatttgaagactgaattaagtgaaattgtactttattttacatatataatgtcatgtacaacttggcttagatagtgtttttgggggggatttttgttttggtgtttatttttacacaaggaaagttgactacttcccattcctggatttaggagaggaactcagaaggcccttaggatgatctgtttgtatgggaagaagttagagacaatggattttataattccagatttattggataaaagctttgtcatttttcagtatcagggaagtaagtcatttaatagcctattttccttaagagtgaactacaggacagtaactatataaatacatgttgatttcttttgcaaaacagttttaagtaatgacatttttgctttatcaaaattttattccatctcttggttttgttgtcaactgccgaaatgtgcgaagctgaaagactgttagcccaagagctgtgtgcactgtgtctttagtgcaggattaccttgtaggttcctagtcctcgaggacttgccagagtgcttatagccacacctggaagtcaggaatgtctccacattatttcaggggattttaggggtcttgtaggctagtcacaaggaagacgacataaccaaaagcacagtaaagttttcgggttaggttcactatgggcttaacagctagaaggattggggggaaagctttagatttaagtaggatcaccagcaaacagctttgtttcagaatggctataagtaggtctaaatgtcttcccaaaaaattggtgtgttttttgcccaggacaaaagtcttaaaggaagttatttaactttttgtttttaaaatgggcttattgttaatgccttatttataaaatgttaaaacaaaatagaatgtgctgggggtgggagctgggcgtgttttccagggctgataacattgaaaactggttcatcctctagtactgagtggttttcaatggcatggctagtcagattcatgatttttaagtataggtgtagattggagcatttattacatattttactgttcaatgtcttaatgtggaccttttattttgtaaataattatggaaatgattgtggggcttggaaaaaattacctttgaagttttaaaagacagtccactttttagcatgtgtgttgtgtccagcctgtggttgtcttaactaataaatgtgatttttctcccc